(19) United States
(12) Patent Application Publication
(10) Pub. No.: US 2026/0167949 A1
(43) Pub. Date: Jun. 18, 2026

(54) FUNGAL PROTEASES, TREATED COMPOSITIONS, AND USES THEREOF

(71) Applicant: Shared-X LLC, Menlo Park, CA (US)

(72) Inventor: Jose Antonio de Cote, Miami, FL (US)

(73) Assignee: Shared-X LLC, Menlo Park, CA (US)

(21) Appl. No.: 19/218,310

(22) Filed: May 25, 2025

Related U.S. Application Data
(63) Continuation of application No. 18/715,615, now abandoned.
(60) Provisional application No. 63/264,931, filed on Dec. 3, 2021.

(51) Int. Cl. C12N 9/58; A01N 63/50; A01P 3/00; A01P 21/00; C12N 9/48; C12P 21/06

(52) U.S. Cl. CPC C12N 9/58; A01N 63/50; A01P 3/00; A01P 21/00; C12N 9/485; C12P 21/06

(57) ABSTRACT

The present disclosure relates to fungal proteases, formulations including proteases, compositions formed by treatment with proteases, as well as methods thereof. In particular, the protease may be obtained from a Trichoderma species.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in their entireties and for all purposes.

### INCORPORATION BY REFERENCE OF SEQUENCE LISTING PROVIDED AS AN XML FILE

The sequence listing of the present application is submitted electronically as ST26 formatted sequence listing XML file “SHARP003WO_2023-02-07_.xml”, file size 350,201 bytes, created on Feb. 7, 2023. This sequence listing submitted is part of the specification and is hereby incorporated by reference in its entirety.

### FIELD

The present disclosure relates to fungal proteases, formulations including proteases, compositions formed by treatment with proteases, as well as methods thereof.

### BACKGROUND

Modern agricultural techniques rely on various chemicals to provide pathogen control and growth stimulation. There is an increased emphasis and awareness of the effects that such chemicals can have, not only on crop production, but also on environmental impact.

The background description provided herein is for the purposes of generally presenting the context of the disclosure. Work of the presently named inventors, to the extent it is described in this background section, as well as aspects of the description that may not otherwise constitute prior art at the time of filing, are neither expressly nor impliedly admitted as prior art against the present disclosure.

### SUMMARY

The present disclosure relates to fungal proteases, which can be provided in a formulation. The protease-containing formulation can include one or more carriers or additives configured for using the protease in hydrolyzing plant proteins or feedstock. Proteases derived from Trichoderma can be of particular use. Thus, in some embodiments, the protease formulation includes one or more Trichoderma proteases. The formulation can include other inert ingredients that do not necessarily provide proteolytic activity. However, such inert ingredients can affect the activity of the proteases. For instance, such inert ingredients can include those that stabilize the protease or enhance activity of the protease upon addition to a feedstock.

A protease formulation, in turn, can be used to treat a feedstock, thereby producing a hydrolysis product. Such hydrolysis products are referred to as compositions and formulations thereof, depending on whether an inert ingredient is added. For example, a hydrolysis product (e.g., as a direct result of feedstock treated with a protease or a protease formulation) is generally referred to as a composition, whereas such a hydrolysis product having an added inert ingredient (e.g., water, oil, etc.) is generally referred to as a formulation.

Thus, also described herein are compositions treated with fungal proteases, as well as methods for preparing and using such compositions. For instance, the composition can be prepared by treating a plant protein or a feedstock with a protease formulation, thereby providing protease-treated components within the hydrolyzed composition. The hydrolyzed composition, in turn, can include one or more other additives (e.g., nutrients), thereby providing a hydrolyzed formulation.

In particular embodiments, the hydrolyzed composition and/or the hydrolyzed formulation have potential as a biostimulant. A biofungicide is a biological control agent that kills or inhibits pathogenic fungi; and the compositions and formulations herein can, in some instances, be a biofungicide or a biological control agent, as described herein. Methods of using such compositions and formulations include delivering the formulation to a plant.

Accordingly, in a first aspect, the present disclosure encompasses a protease formulation including: one or more proteases from a Trichoderma species; and one or carriers and/or additives. In some embodiments, the formulation is configured to hydrolyze a plant protein or a feedstock.

In some embodiments, the protease is an alkaline proteinase, a serine protease, a subtilisin-like protease, a trypsin-like serine protease, an aspartyl protease, a metallopeptidase, a carboxypeptidase, a glutamate/glutamine protease, a cysteine protease, or the like, as well as any described herein. In other embodiments, the protease includes a polypeptide sequence having at least 80%, 85%, 90%, 95%, 96%, 97%, 98%, 99%, or greater sequence identity to any one of the following SEQ ID NOs:1-18, 20-30, 40-51, 60-73, 80-87, 90-94, and 100-107. In yet other embodiments, the protease includes a polypeptide sequence having at least 80%, 85%, 90%, 95%, 96%, 97%, 98%, 99%, or greater sequence identity to any one of the proteins associated with the UniProtKB Entry numbers in Tables 1-5.

In some embodiments, the Trichoderma species is T. parareesei, T. virens, T. atroviride, and/or T. asperellum. In other embodiments, the Trichoderma species is T. parareesei strain T6, T. virens strain T59, T. atroviride strain T11, or T. asperellum strain T25.

In some embodiments, the one or more carriers includes a liquid carrier or a solid carrier. In some embodiments, the one or more additives includes a metabolic inhibitor, a stabilizer, and/or a nutrient.

In a second aspect, the present disclosure encompasses a method of preparing a hydrolyzed composition. Such a method can include: receiving a plant-based feedstock including a plant protein; and hydrolyzing the plant protein with a protease to produce a hydrolysis product including an amino acid and/or an oligopeptide, wherein the protease is from a Trichoderma species.

In some embodiments, the hydrolysis product is a biofungicide composition. In some embodiments, the plant-based feedstock is selected from legumes, tarwi, peanut, carob germ, soybean, and Plukenetia volubilis, as well as any described herein.

In some embodiments, said hydrolyzing includes introducing a protease formulation (e.g., any described herein) to the plant-based feedstock. In other embodiments, said hydrolyzing includes introducing an isolate including the Trichoderma species to the plant protein.

In some embodiments, said hydrolyzing includes introducing the protease to the plant-based feedstock. Non-limiting examples of proteases include an alkaline proteinase, a serine protease, a subtilisin-like protease, a trypsin-like serine protease, an aspartyl protease, a metallopeptidase, a carboxypeptidase, a glutamate/glutamine protease, a cysteine protease, or any described herein. In some embodiments, the protease includes a polypeptide sequence having at least 90% sequence identity to any one of the following SEQ ID NOs:1-18, 20-30, 40-51, 60-73, 80-87, 90-94, and 100-107. In other embodiments, the protease includes a polypeptide sequence having at least 80%, 85%, 90%, 95%, 96%, 97%, 98%, 99%, or greater sequence identity to any one of the proteins associated with the UniProtKB Entry numbers in Tables 1-5.

In some embodiments, the amino acid is selected from glutamic acid, glutamine, glycine, threonine, alanine, leucine, lysine, and combinations thereof. In some embodiments, the amino acid is present in a trace amount, wherein the amino acid is selected from aspartic acid, serine, tyrosine, arginine, valine, tryptophan, phenylalanine, asparagine, isoleucine, histidine, methionine, glutamine, proline, hydroxyproline, omithine, taurine, and combinations thereof.

In some embodiments, the hydrolysis product further includes one or more phytohormones. Non-limiting examples of one or more phytohormones include cytokinins, abscisic acids (ABAs), jasmonates, auxins, phenolics, as well as any described herein and combinations of any of these.

In some embodiments, the method further includes: adding one or more nutrients to the plant-based feedstock or the hydrolysis product, thereby providing a biostimulant formulation. In particular embodiments, the one or more nutrients are selected from calcium, potassium, sulfur, magnesium, carbon, oxygen, hydrogen, iron, manganese, boron, molybdenum, zinc, chlorine, sodium, and cobalt.

In a third aspect, the present disclosure encompasses a composition (e.g., a biostimulant composition or a biofungicidal composition including: a hydrolysis product including an amino acid and an oligopeptide, wherein the amino acid and the oligopeptide are derived from a plant-based feedstock; and a trace amount of a protease, or fragments thereof, from a Trichoderma species. In some embodiments, the amino acid is selected from glutamic acid, glutamine, glycine, threonine, alanine, leucine, lysine, and combinations thereof. In other embodiments, the amino acid is present in a trace amount, wherein the amino acid is selected from aspartic acid, serine, tyrosine, arginine, valine, tryptophan, phenylalanine, asparagine, isoleucine, histidine, methionine, glutamine, proline, hydroxyproline, omithine, taurine, and combinations thereof.

In particular embodiments, the hydrolysis product further includes one or more phytohormones. In other embodiments, the one or more phytohormones are selected from the group consisting of cytokinins, abscisic acids (ABAs), jasmonates, auxins, and phenolics.

In some embodiments, the trace amount of the protease includes a trace amount of an alkaline proteinase, a serine protease, a subtilisin-like protease, a trypsin-like serine protease, an aspartyl protease, a metallopeptidase, a carboxypeptidase, a glutamate/glutamine protease, or a cysteine protease. In other embodiments, the protease includes a polypeptide sequence having at least 80%, 85%, 90%, 95%, 96%, 97%, 98%, 99%, or greater sequence identity to any one of the following SEQ ID NOs:1-18, 20-30, 40-51, 60-73, 80-87, 90-94, and 100-107. In yet other embodiments, the protease includes a polypeptide sequence having at least 80%, 85%, 90%, 95%, 96%, 97%, 98%, 99%, or greater sequence identity to any one of the proteins associated with the UniProtKB Entry numbers in Tables 1-5.

In some embodiments, the trace amount of the protease includes a trace amount of a protease from T. parareesei, T. virens, T. atroviride, and/or T. asperellum. In other embodiments, the Trichoderma species is T. parareesei strain T6, T. virens strain T59, T. atroviride strain T11, or T. asperellum strain T25.

In a fourth aspect, the present disclosure encompasses a formulation (e.g., a biostimulant formulation or a biofungicidal formulation) including: a composition (e.g., any described herein, including a biostimulant composition or a biofungicidal composition); and one or more nutrients. In some embodiments, the one or more nutrients include a micronutrient and/or a macronutrient. In other embodiments, the one or more nutrients are selected from calcium, potassium, sulfur, magnesium, carbon, oxygen, hydrogen, iron, manganese, boron, molybdenum, zinc, chlorine, sodium, and cobalt. In yet other embodiments, the formulation further includes water.

In a fifth aspect, the present disclosure encompasses a method of treating a plant. In some embodiments, the method includes: preparing a composition (e.g., any described herein) or a formulation (e.g., any described herein); and delivering the composition or the formulation to the plant, a portion thereof, a plant material, or a soil in proximity to the plant.

In some embodiments, the method further includes (e.g., prior to said delivering): providing the composition or the formulation to an irrigation system configured to irrigate the plant. In other embodiments, the method further includes (e.g., prior to said delivering): providing the composition or the formulation to a mister.

In some embodiments, said treating includes protecting the plant against a pathogenic organism, stimulating growth of the plant, or counteracting growth of a pathogenic organism in proximity to the plant. In further embodiments, said delivering includes delivering an effective amount of the composition or the formulation for said treating.

In any embodiment herein, the Trichoderma species is T. parareesei, T. virens, T. atroviride, and/or T. asperellum. In some embodiments, the Trichoderma species is T. parareesei strain T6, T. virens strain T59, T. atroviride strain T11, or T. asperellum strain T25. Additional details follow.

### Definitions

“Biostimulant composition” or “nutritional corrector composition” may refer to a composition, which may be a substance or mixture, that supplements or corrects nutritional deficiencies in a plant to improve the function of the plant by stimulating biological processes, improving the availability of nutrients, optimizing the plants' absorption of nutrients, increase tolerance to abiotic stresses, and/or improve quality aspects of the harvest.

“Amino acid profile” may refer to the amounts of the amino acids present in a composition. Amino acid profiles may be qualitative or quantitative. Qualitative amino acid profiles identify which amino acids are present in a composition. Quantitative amino acid profiles refer to the relative amounts of amino acids present in a composition and/or to the absolute amounts of amino acids present in a composition.

“Free amino acid” or “free amino acid component” may refer to an amino acid that is not bound to other amino acids and/or peptides via peptide bonds.

A “primary amino acid component” may refer to an amino acid in a composition that is at least about 1% (w/w) of the total weight of amino acids in a composition. In some embodiments, a primary amino acid component is at least about 10% (w/w) of the total weight of amino acids in a composition.

A “secondary amino acid component” may refer to an amino acid in a composition that has a concentration of less than about 1% (w/w) of the total weight of amino acids in a composition. In some embodiments, a secondary amino acid component is greater than about 0.01% and less than 0.7% (w/w) of total weight of amino acids in a composition.

“Feedstock” may refer to a raw, unprocessed material source that can be processed and/or broken down to generate nutritional components.

“Enzymatic hydrolysis” may refer to a process which enzymes are used to facilitate degradation of a feedstock by hydrolytically cleaving bonds in molecules with the addition of the elements of water. Proteases are sometimes used to perform enzymatic hydrolysis on a protein-containing feedstock.

“Micronutrient” may refer to a secondary plant nutrient used in smaller amounts for nourishment and growth of a plant. A plant nutrient is secondary if a plant only uses trace amounts of it to sustain life. Examples of micronutrients include iron, manganese, zinc, copper, boron, and molybdenum.

“Macronutrient” may refer to a plant nutrient used in large amounts for nourishment and growth of a plant. Examples of primary macronutrients are nitrogen, phosphorous, and potassium. Examples of secondary macronutrients are magnesium, sulfur, and calcium.

As used herein, a “biological control agent” may be a biologically-derived agent, composition, or formulation that counteracts the growth of pathogenic microbes and/or that counteracts the effect of pathogenic microbes on a plant or a plant material. A non-limiting example of a biological control agent (BCA) includes a biofungicide that kills or inhibits pathogenic fungi. A BCA can also exhibit other properties, such as for a biostimulant that stimulates plant growth. Such BCAs can be derived from a biological source by, for example, culturing a microorganism and obtaining components from culture media, fermentation broth, supernatant, and like by using isolation and separation techniques; or by, for example, treating a plant protein or a feedstock with a protein obtained from a microorganism, thereby obtaining protein-treated components within a composition; or by, for example, treating a plant protein or a feedstock with a microorganism, thereby obtaining microorganism-treated components within a composition. Non-limiting examples of components can include a protein, peptide, or amino acid derived from a biological source (e.g., a microorganism) or treated with a biological source (e.g., a microorganism); a compound derived from a biological source (e.g., a microorganism); an isolate from a culture having a biological source (e.g., a microorganism, such as a bacterium, a virus, or a fungus); a spore (e.g., obtained from an isolate); mycelium (e.g., obtained from an isolate); and the like.

As used herein, a “carrier” may be solid or liquid and may include substances ordinarily employed in formulations applied to plants. Carriers may include any described herein, such as binders, encapsulating materials, carbonaceous matter, fillers, desiccants, liquids (e.g., water), dispersants, as well as combinations thereof. The carrier can provide a formulation that is a liquid, gel, slurry, or solid. Further examples of carriers are described herein.

As used herein, an “effective amount” refers to a sufficient amount to obtain beneficial or desired result(s). An effective amount can be administered in a single operation or in several administrations. In terms of treatment or protection, a “sufficient amount” is the amount to palliate, improve, stabilize, revert, retard, or delay the progression of the stages of disease caused by a pathogen. In some embodiments, an effective amount is intended to mean an amount of a formulation described herein sufficient to inhibit the growth of a microorganism on a plant by, for example, 10%, 20%, 50%, 75%, 80%, 90%, 95%, or more; or 1-fold, 3-fold, 5-fold, 10-fold, 20-fold, or more, as compared to a negative control plant not treated with a formulation or a composition provided herein.

As used herein, an “isolate” means a separated or isolated culture that includes a microorganism, as well as a portion of such a culture. Non-limiting microorganisms include a bacteria, a virus, or a fungus, as well as particular species for such microorganisms. An isolate can include one or more different components, such as spores, mycelium, proteins, nutrients, as well as combinations thereof. Isolates can be derived from a microorganism by, for example, culturing the microorganism and obtaining components from culture media (which may be a solid or a liquid), fermentation broth, supernatant, and the like by using isolation and/or separation techniques. The isolate can include components from the same microorganism, from different species of a microorganism, or from different microorganisms. As described herein, a non-limiting example of an isolate includes a Trichoderma isolate. Such a Trichoderma isolate can include one or more components obtained from a culture including one or more Trichoderma species; or obtained by combining components from two or more cultures, in which at least one culture includes one or more Trichoderma species.

The terms “polynucleotide” and “nucleic acid,” used interchangeably herein, refer to a polymeric form of nucleotides of any length, either ribonucleotides or deoxyribonucleotides. Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, DNA-RNA hybrids, or a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2(4):482-9) as incorporated into GeneMatcher Plus™ (Schwarz and Dayhof, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to an uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis., 53705). Such software may match similar sequences by assigning degrees of homology to various substitutions, deletions, and other modifications. The present disclosure encompasses a polypeptide or nucleic acid sequence that is substantially identical to any described herein.

By “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones. Non-limiting amino acids include glycine (Gly, G), alanine (Ala, A), valine (Val, V), isoleucine (Ile, I), leucine (Leu, L), cysteine (Cys, C), methionine (Met, M), aspartic acid (Asp, D), glutamic acid (Glu, E), arginine (Arg, R), histidine (His, H), lysine (Lys, K), asparagine (Asn, N), glutamine (Gln, Q), serine (Ser, S), threonine (Thr, T), proline (Pro, P), phenylalanine (Phe, F), tyrosine (Tyr, Y), tryptophan (Trp, W), selenocysteine (Sec, U), and pyrrolysine (Pyl, O).

A “peptide” may refer to a linear chain of amino acids linked by amide-type chemical bonds, which are called peptide bonds. Thus, to form peptides, amino acids are linked together forming chains of variable length and sequence. Dipeptides may refer to a linear chain of two amino acids linked by a peptide bond. Tripeptides may refer to a linear chain of three amino acids, and tetrapeptides may refer to a linear chain of four amino acids.

An “oligopeptide” may refer to a peptide having less than 10 amino acids.

The term “modified,” as used in reference to amino acids, means an amino acid including one or more modifications, such as a post-translation modification (e.g., acetylation, methylation, phosphorylation, ubiquitination, sumoylation, ribosylation, glycosylation, acylation, or isomerization), or including a non-natural amino acid.

The term “modified,” as used in reference to a protein, means a polypeptide sequence including one or more amino acid substitution, as compared to the reference sequence for the protein.

The term “fragment” is meant a portion of a nucleic acid or a polypeptide that is at least one nucleotide or one amino acid shorter than the reference sequence. This portion contains, preferably, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides; or 10, 20, 30, 40, 50, 60, 70, 80, 90, 100, 150, 200, 250, 300, 350, 400, 450, 500, 550, 600, 640 amino acids or more. In another example, any polypeptide fragment can include a stretch of at least about 5 (e.g., about 10, about 20, about 30, about 40, about 50, or about 100) amino acids that are at least about 40% (e.g., about 50%, about 60%, about 70%, about 80%, about 90%, about 95%, about 87%, about 98%, about 99%, or about 100%) identical to any of the sequences described herein can be utilized in accordance with the invention. In certain embodiments, a polypeptide to be utilized in accordance with the invention includes 2, 3, 4, 5, 6, 7, 8, 9, 10, or more mutations (e.g., one or more conservative amino acid substitutions, as described herein). In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the invention.

The term “conservative amino acid substitution” refers to the interchangeability in proteins of amino acid residues having similar side chains (e.g., of similar size, charge, and/or polarity). For example, a group of amino acids having aliphatic side chains consists of glycine (Gly, G), alanine (Ala, A), valine (Val, V), leucine (Leu, L), and isoleucine (Ile, I); a group of amino acids having aliphatic-hydroxyl side chains consists of serine (Ser, S) and threonine (Thr, T); a group of amino acids having amide containing side chains consisting of asparagine (Asn, N) and glutamine (Gln, Q); a group of amino acids having aromatic side chains consists of phenylalanine (Phe, F), tyrosine (Tyr, Y), and tryptophan (Trp, W); a group of amino acids having basic side chains consists of lysine (Lys, K), arginine (Arg, R), and histidine (His, H); a group of amino acids having acidic side chains consists of glutamic acid (Glu, E) and aspartic acid (Asp, D); a group of polar amino acids consists of D, E, N, and Q; and a group of amino acids having sulfur containing side chains consists of cysteine (Cys, C) and methionine (Met, M). Exemplary conservative amino acid substitution groups are valine-leucine-isoleucine (VLI), phenylalanine-tyrosine (FY), lysine-arginine (KR), alanine-valine (AV), glycine-serine (GS), glutamate-aspartate (ED), and asparagine-glutamine (NQ). The present disclosure encompasses any sequence having a conservative amino acid sequence of any polypeptide sequence described herein. Additional details follow.

### BRIEF DESCRIPTION OF THE DRAWINGS

FIG. 1A-1B shows schematic illustration of components of a formulation in accordance with certain disclosed embodiments. Provided are formulations including (A) amino acids 120a/120b and oligopeptides 130; and (B) further including macronutrients 140 and micronutrients 150a/150b/150c.

FIG. 2A-2B shows process flow diagram depicting operations performed in a method performed in accordance with certain disclosed embodiments. Provided are methods including (A) performing enzymatic hydrolysis on plant protein or feedstock and (B) further operations to provide a biostimulant formulation.

FIG. 3 is a process flow diagram depicting operations performed in a method performed in accordance with certain disclosed embodiments.

FIG. 4A-4B shows schematic illustrations depicting example techniques for applying a formulation in accordance with certain disclosed embodiments. Provided are techniques including (A) use of an irrigation system and (B) use of manual application to provide the formulation.

FIG. 5 is a schematic illustration of an enzymatic hydrolysis reactor that may be used to perform certain disclosed embodiments.

FIG. 6 is process flow diagram depicting operations for providing a biostimulant with an optional fungicide.

FIG. 7A-7C shows non-limiting amino acid sequences for alkaline proteinases or serine proteases. Provided are sequences for Hypocrea atroviridis (Q03420, SEQ ID NO:1); T. virens (Q874K4, SEQ ID NO:2); T. hamatum (Q86ZV3, SEQ ID NO:3); T. harzianum (A0A0F9X8B4, SEQ ID NO:4); T. gamsii (A0A2P4ZGM1, SEQ ID NO:5); T. guizhouense (A0A1T3C9N5, SEQ ID NO:6); T. asperellum (A0A6V8QT77, SEQ ID NO:7); T. arundinaceum (A0A395NJ00, SEQ ID NO:8); T. asperellum (A0A6V8QUL4, SEQ ID NO:9); T. atroviride (G9P6E2, SEQ ID NO:10); T. koningiopsis (A0A5B8WCX7, SEQ ID NO:11); and T. reesei (G0RHA8, SEQ ID NO:12). Also provided are consensus sequences, including Cons1 (SEQ ID NO:13), Cons2 (SEQ ID NO:14), Cons3 (SEQ ID NO:15), Cons4 (SEQ ID NO:16), Cons5 (SEQ ID NO:17), and Cons6 (SEQ ID NO:18). In another embodiment, for each consensus sequence (SEQ ID NOs:13-18), each X (if present) at each position is an amino acid (or a modified form thereof) that is provided in an aligned reference sequence. For instance, this X can be any amino acid provided in an aligned reference sequence (e.g., aligned reference sequences SEQ ID NOs:1-12 for the consensus sequence in one of SEQ ID NOs:13-18). In the alignment, an * (asterisk) indicates positions which have a single, fully conserved residue. Furthermore, a : (colon) indicates conservation between groups of strongly similar properties roughly equivalent to scoring >0.5 in the Gonnet PAM 250 matrix. Non-limiting groups of amino acids having strongly similar properties include, e.g., serine-threonine-alanine (STA); asparagine-glutamate-glutamine-lysine (NEQK); asparagine-histidine-glutamine-lysine (NHQK); asparagine-aspartate-glutamate-glutamine (NDEQ); glutamine-histidine-arginine-lysine (QHRK); methionine-isoleucine-leucine-valine (MILV); methionine-isoleucine-leucine-phenylalanine (MILF); histidine-tyrosine (HY); and phenylalanine-tyrosine-tryptophan (FYW). A . (period) indicates conservation between groups of weakly similar properties as roughly equivalent to scoring=<0.5 and >0 in the Gonnet PAM 250 matrix. Non-limiting groups of amino acids having weakly similar properties include, e.g., cysteine-serine-alanine (CSA); alanine-threonine-valine (ATV); serine-alanine-glycine (SAG); serine-threonine-asparagine-lysine (STNK); serine-threonine-proline-alanine (STPA); serine-glycine-asparagine-aspartate (SGND); serine-asparagine-aspartate-glutamate-glutamine-lysine (SNDEQK); asparagine-aspartate-glutamate-glutamine-histidine-lysine (NDEQHK); asparagine-glutamate-glutamine-histidine-arginine-lysine (NEQHRK); phenylalanine-valine-leucine-isoleucine-methionine (FVLIM); and histidine-phenylalanine-tyrosine (HFY).

FIG. 8A-8C shows non-limiting amino acid sequences for subtilisin-like proteases or serin endopeptidases. Provided are sequences for T. harzianum (A4V8W5, SEQ ID NO:20); Hypocrea virens (strain Gv29-8/FGSC 10586) (G9MWK6, SEQ ID NO:21); Hypocrea jecorina (strain QM6a) (G0RT14, SEQ ID NO:22); T. parareesei (A0A2H2Z2P7, SEQ ID NO:23); and T. longibrachiatum ATCC 18648 (A0A2T4C1T9, SEQ ID NO:24). Also provided are consensus sequences, including Cons1 (SEQ ID NO:25), Cons2 (SEQ ID NO:26), Cons3 (SEQ ID NO:27), Cons4 (SEQ ID NO:28), Cons5 (SEQ ID NO:29), and Cons6 (SEQ ID NO:30). In another embodiment, for each consensus sequence (SEQ ID NOs:25-30), each X (if present) at each position is an amino acid (or a modified form thereof) that is provided in an aligned reference sequence. For instance, this X can be any amino acid provided in an aligned reference sequence (e.g., aligned reference sequences SEQ ID NOs:20-24 for the consensus sequence in one of SEQ ID NOs:25-30). The symbols for * (asterisk), : (colon), and . (period) refer to the same groups as described above for FIG. 7A-7C.

FIG. 9A-9B shows non-limiting amino acid sequences for trypsin-like serine proteases. Provided are sequences for T. harzianum (Q8WZM5, SEQ ID NO:40); Hypocrea jecorina (strain QM6a) (G0R816, SEQ ID NO:41); T. guizhouense (A0A1T3C4V6, SEQ ID NO:42); T. guizhouense (A0A1T3CMG9, SEQ ID NO:43); T. harzianum (G8G0S6, SEQ ID NO:44); T. longibrachiatum ATCC 18648 (A0A2T4CCJ3, SEQ ID NO:45); T. parareesei (A0A2H3A454, SEQ ID NO:46); T. citrinoviride (A0A2T4BDN7, SEQ ID NO:47); and T. arundinaceum (A0A395NV63, SEQ ID NO:48). Also provided are consensus sequences, including Cons1 (SEQ ID NO:49), Cons2 (SEQ ID NO:50), and Cons3 (SEQ ID NO:51). In another embodiment, for each consensus sequence (SEQ ID NOs:49-51), each X (if present) at each position is an amino acid (or a modified form thereof) that is provided in an aligned reference sequence. For instance, this X can be any amino acid provided in an aligned reference sequence (e.g., aligned reference sequences SEQ ID NOs:40-48 for the consensus sequence in one of SEQ ID NOs:49-51). The symbols for * (asterisk), : (colon), and . (period) refer to the same groups as described above for FIG. 7A-7C.

FIG. 10A-10C shows non-limiting amino acid sequences for aspartyl proteases, aspartate proteases, aspartic protease pro-precursors, endothiapepsins, or peptidase A1 domain-containing proteins. Provided are sequences for T. harzianum (A0A2K0UA10, SEQ ID NO:60); Hypocrea atroviridis (strain ATCC 20476/IMI 206040) (G9NK25, SEQ ID NO:61); T. gamsii (A0A2K0T047, SEQ ID NO:62); Hypocrea jecorina (Q2WBH2, SEQ ID NO:63); Hypocrea virens (strain Gv29-8/FGSC 10586) (G9MLM6, SEQ ID NO:64); T. harzianum (Q9HDT6, SEQ ID NO:65); T. parareesei (A0A2H2ZPA8, SEQ ID NO:66); T. citrinoviride (A0A2T4BEG6, SEQ ID NO:67); T. orientale (I2EC21, SEQ ID NO:68); T. arundinaceum (A0A395NB42, SEQ ID NO:69); and T. asperellum (Q64ID0, SEQ ID NO:70). Also provided are consensus sequences, including Cons1 (SEQ ID NO:71), Cons2 (SEQ ID NO:72), and Cons3 (SEQ ID NO:73). In another embodiment, for each consensus sequence (SEQ ID NOs:71-73), each X (if present) at each position is an amino acid (or a modified form thereof) that is provided in an aligned reference sequence. For instance, this X can be any amino acid provided in an aligned reference sequence (e.g., aligned reference sequences SEQ ID NOs:60-70 for the consensus sequence in one of SEQ ID NOs:71-73). The symbols for * (asterisk), : (colon), and . (period) refer to the same groups as described above for FIG. 7A-7C.

FIG. 11A-11B shows non-limiting amino acid sequences for aspartyl proteases and peptidase A1 domain-containing proteins. Provided are sequences for T. harzianum (Q334I5, SEQ ID NO:80); T. harzianum CBS 226.95 (A0A2T4A8V4, SEQ ID NO:81); T. harzianum (A0A2K0TFV3, SEQ ID NO:82); T. asperellum (Q64HW0, SEQ ID NO:83); and Hypocrea virens (strain Gv29-8/FGSC 10586) (G9MS93, SEQ ID NO:84). Also provided are consensus sequences, including Cons1 (SEQ ID NO:85), Cons2 (SEQ ID NO:86), and Cons3 (SEQ ID NO:87). In another embodiment, for each consensus sequence (SEQ ID NOs:85-87), each X (if present) at each position is an amino acid (or a modified form thereof) that is provided in an aligned reference sequence. For instance, this X can be any amino acid provided in an aligned reference sequence (e.g., aligned reference sequences SEQ ID NOs:80-84 for the consensus sequence in one of SEQ ID NOs:85-87). The symbols for * (asterisk), : (colon), and . (period) refer to the same groups as described above for FIG. 7A-7C.

FIG. 12 shows non-limiting amino acid sequences for metallopeptidases, zincins, or M43 domain-containing proteins. Provided are sequences for Hypocrea atroviridis (strain ATCC 20476/IMI 206040) (G9NHX6, SEQ ID NO:90); T. gamsii (A0A0W7VXN4, SEQ ID NO:91); and T. longibrachiatum ATCC 18648 (A0A2T4CEU0, SEQ ID NO:92). Also provided are consensus sequences, including Cons1 (SEQ ID NO:93) and Cons2 (SEQ ID NO:94). In another embodiment, for each consensus sequence (SEQ ID NOs:93-94), each X (if present) at each position is an amino acid (or a modified form thereof) that is provided in an aligned reference sequence. For instance, this X can be any amino acid provided in an aligned reference sequence (e.g., aligned reference sequences SEQ ID NOs:90-92 for the consensus sequence in one of SEQ ID NOs:93-94). The symbols for * (asterisk), : (colon), and . (period) refer to the same groups as described above for FIG. 7A-7C.

FIG. 13A-13B shows non-limiting amino acid sequences for carboxypeptidases or M14 domain-containing proteins. Provided are sequences for Hypocrea atroviridis (strain ATCC 20476/IMI 206040) (G9P8Z8, SEQ ID NO:100); Hypocrea virens (strain Gv29-8/FGSC 10586) (G9MKD8, SEQ ID NO:101); T. harzianum CBS 226.95 (A0A2T3ZS57, SEQ ID NO:102); T. harzianum (A0A2K0ULU3, SEQ ID NO:103); T. parareesei (A0A2H2ZH18, SEQ ID NO:104); and T. harzianum (A0A0F9ZWU1, SEQ ID NO:105). Also provided are consensus sequences, including Cons1 (SEQ ID NO:106) and Cons2 (SEQ ID NO:107). In another embodiment, for each consensus sequence (SEQ ID NOs:106-107), each X (if present) at each position is an amino acid (or a modified form thereof) that is provided in an aligned reference sequence. For instance, this X can be any amino acid provided in an aligned reference sequence (e.g., aligned reference sequences SEQ ID NOs:100-105 for the consensus sequence in one of SEQ ID NOs:106-107). The symbols for * (asterisk), : (colon), and . (period) refer to the same groups as described above for FIG. 7A-7C.

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

Agricultural crop generation involves consideration of various factors to ensure healthy and productive growth of the crops, including the geographical location and growth conditions. However, crops may encounter various agricultural growth difficulties, including soil contamination, genetic mutations, pests (such as insects), disease (e.g., fungal, bacterial, and viral diseases), disruptive effects of automated techniques (e.g., tilling, planting, harvesting, watering, etc.), and other non-ideal growing conditions such as soil composition, humidity (excessive or very low), temperature (very high or very low), luminosity level (e.g., excess solar luminosity or lack thereof), flooding and/or drought, stress caused by fertilizers, inadequate pollination, excess of soil salts (e.g., minerals), and lack of organic material and/or minerals in the soil.

In one embodiment, it is useful to use substances that act as a biological control agent (BCA). For instance, such an agent can counteract the growth or effect of pathogenic microbes on a plant or a plant material. In one example, a BCA includes a biofungicide that kills or inhibits pathogenic fungi. A BCA can also exhibit other properties, such as for a biostimulant that stimulates plant growth.

In another embodiment, to help resolve agricultural growth difficulties, it is useful to use substances that are compatible with the plants. One type of compatibility that may be used considers the types of amino acids that the plant generates to sustain life. During the agricultural growth process, plants spend energy manufacturing certain amino acids that are important for their well-being. Biostimulants and/or nutritional correctors can supply these amino acids and allow the plant to redirect its energy to performing other functions. Application of biostimulants may reduce negative impacts of biotic stressors as well as abiotic stressors and help correct micronutrient and/or macronutrient deficiencies in the plant. Biostimulant compositions described herein have an amino acid profile. That profile may be based, at least in part, on an initial feedstock used to make the biostimulant composition. Example feedstocks include plant waste (e.g., husks or seedpods) and plants having limited economic value.

It has been observed that some biostimulant compositions, such as those derived from rice, do not have an amino acid profile that matches the needs of some plants growing under some conditions. Additionally, some biostimulant compositions are created using acid hydrolysis, which often destroys certain nutrients and/or amino acids in the feedstock, which can generate free amino acids that may be useful to a plant. While animal derived biostimulants may be generated, such biostimulant compositions lack some components such as phytohormones that are beneficial for plant growth. Further, it is generally more difficult to break down proteins from animal feedstock than from plant feedstock. Some biostimulant compositions may also not be suitable because of synthesis difficulties, lack of efficiency in generating the composition, cost of production, and environmental condition limitations.

Preparing such biofungicides or biostimulants can be challenging, and this present disclosure relates to proteases from one or more Trichoderma species for enzymatic hydrolysis of the feedstock. In particular, the protease can be provided in a formulation (e.g., a protease formulation) configured to hydrolyze a plant protein or a feedstock including the plant protein. The protease can be isolated from a Trichoderma species prior to being introduced to the feedstock. Alternatively, an isolate including the Trichoderma species can be provided to the feedstock, so long as the isolate provides or expresses the desired protease.

In certain embodiments, the feedstock contains plant material such material from a carob plant, a peanut plant, a lupin plant, a soybean plant, a rice plant, or the like. Sources that have a high concentration of vegetable protein can be used in various embodiments. When biostimulant compositions or biofungicidal compositions are produced from plant feedstock, acid hydrolysis is not used. Some disclosed biostimulant compositions are produced by enzymatic hydrolysis of plant feedstock.

The protease can be used to enzymatically treat the feedstock, thereby providing a hydrolysis product. The protease may be provided as a formulation, which can include any useful carrier or additive, such as any described herein. For example, the protease (or mixture of proteases) may be provided in liquid form or solid form (e.g., lyophilized form). In use, the protease formulation can be added to the feedstock and then incubated under conditions to promote proteolytic cleavage of plant proteins present within the feedstock, thereby providing a liquid-based hydrolysis product.

The hydrolysis product can include multiple amino acids, in one instance. Other components may be present, such as one or more components that act as a secondary metabolite. Certain disclosed biostimulant compositions can include oligopeptides that may bioencapsulate micronutrients and/or macronutrients. In certain embodiments, biostimulant compositions are produced from feedstocks that generate an amino acid profile suitable for plants of many types.

The result from enzymatic hydrolysis of feedstock may include free amino acids, peptides, and proteins. Free amino acids can be obtained from hydrolysis and are not bound to any other amino acids through peptide bonds. Due to the low molecular weight of free amino acids, plants are able to assimilate free amino acids quickly and their effects on plant metabolism are more defined. Therefore, free amino acids can be important in plant nutrition. Of note, when two or more amino acids are joined together (by a peptic bond), they form a peptide. The longer the length of the peptide (more amino acids attached), the more difficult will be the direct assimilation by plants. In addition to free amino acids, peptides or proteins may be present within the liquid hydrolysis product. The union of the different polypeptide chains forms a protein. The structural units of proteins are the amino acids joined in a sequence and the characteristic order for each type of protein. Free amino acids and some low molecular weight peptides are useful as products applied to plants. The percentage of each type of amino acids depends on the type of hydrolysate and the origin of the proteins (animal or vegetable), and with it, the quality of the final product.

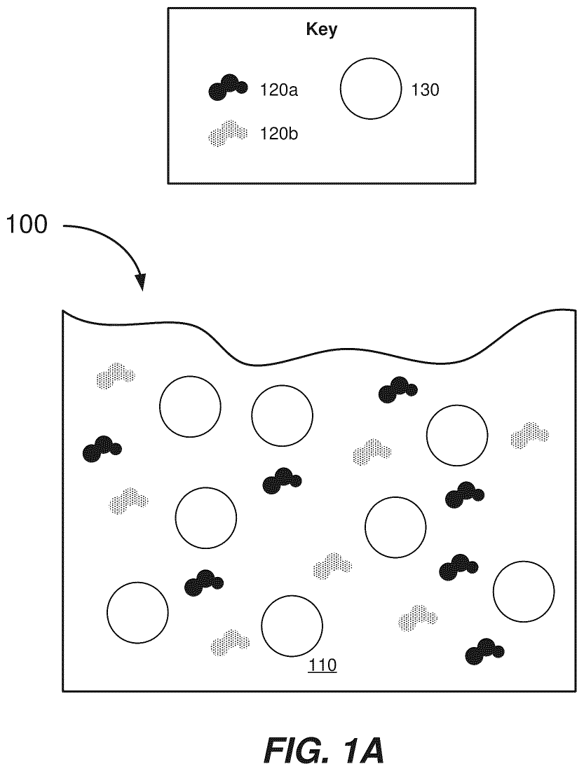

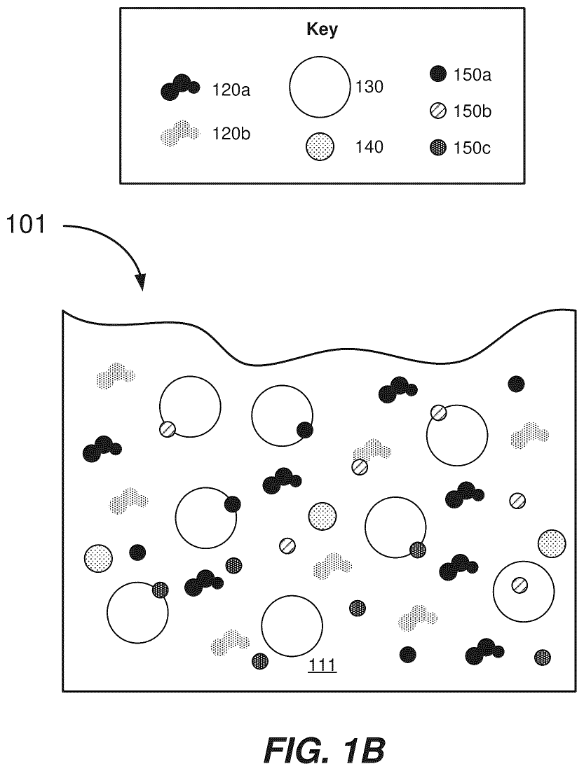

FIG. 1A shows a non-limiting schematic illustration of components within a hydrolysis product. Provided is a composition 100 having a liquid 110 with suspended components. The suspended components include various types of free amino acids, which are depicted as a first type of amino acid 120a and a second type of amino acid 120b. Although two types are depicted in this figure, it will be understood by a person of skill in the art that many types of free amino acids may be in the liquid 110 depending on the amino acid profile, and that the relative concentrations of the free amino acids may vary. The liquid 110 can also include oligopeptides 130, in which enzymatic treatment may not completely hydrolyze the entire protein into fee amino acids.

The hydrolysis product, in turn, can be employed as a biostimulant composition and can include other additives (e.g., nutrients) to provide a formulation (e.g., a biostimulant formulation). Such formulations can stimulate plant growth, such as for a biostimulant. In other instances, the compositions and formulations herein can provide biotic stress relief, in which, for example, the plant might be capable of continuing production even when under attack by a pathogen.

For instance, FIG. 1B shows an example of a biostimulant formulation or a biofungicidal formulation including various components. FIG. 1B includes a formulation 101 having a liquid 111 with suspended components. The suspended components include various types of free amino acids, which are depicted as a first type of amino acid 120a and a second type of amino acid 120b. As described above, many types of free amino acids may be in the liquid depending on the amino acid profile, and the relative concentrations of the free amino acids may vary. The liquid 111 also includes macronutrients 140, as well as micronutrients 150a/150b/150c. Although one type of macronutrient and three types of micronutrients are depicted in this figure, it will be understood by a person of skill in the art that more or fewer types of micronutrients and more or fewer types of macronutrients may be presented in the composition 101.

In various embodiments, manganese, boron, zinc, and mixtures of zinc and manganese may be one or more of micronutrients 150a/150b/150c. In some embodiments, only one type of micronutrient (e.g., manganese, boron, or zinc) is present. In some embodiments, mixtures of micronutrients (e.g., zinc and manganese) are present. In various embodiments, calcium or potassium may be macronutrient 140. In some embodiment, only one type of macronutrient is added; and no additional micronutrients 150a/150b/150c are added, but some micronutrients from the original feedstock itself may be present. In some embodiments, some micronutrients 150a/150b/150c and/or macronutrients 140 may be derived from the plant-based protein source. In some embodiments, some micronutrients 150a/150b/150c and/or macronutrients 140 may be subsequently added to the liquid 111. The liquid 111 can also include oligopeptides 130, which may bioencapsulate micronutrients 150a/150b/150c to help facilitate their delivery to parts of a plant.

The compositions and formulations herein can be prepared by providing a feedstock and then combining one or more proteases from a Trichoderma strains or species with the feedstock, thereby obtaining a hydrolysis product. The hydrolysis product may be further treated, such as by separating one or more components from the hydrolysis product and optionally combining the component(s) with a nutrient.

The compositions and formulations herein can be prepared using any of various methods. In some embodiments, the compositions are made by conducting enzymatic hydrolysis of a plant-based protein source and by optionally adding supplemental nutrients to the composition, either before or after the hydrolysis. The enzymatic hydrolysis converts plant-based protein to free amino acids and oligopeptides.

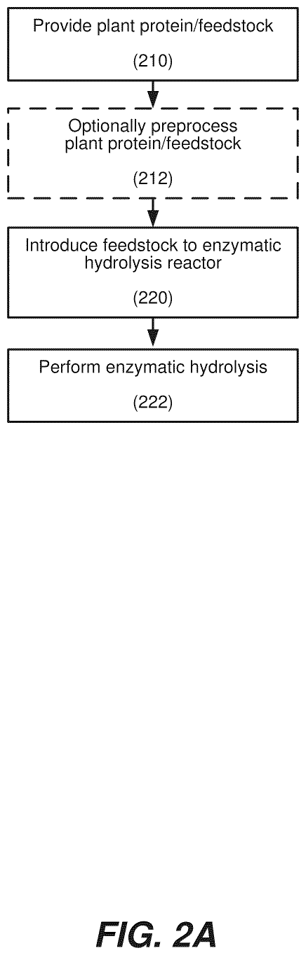

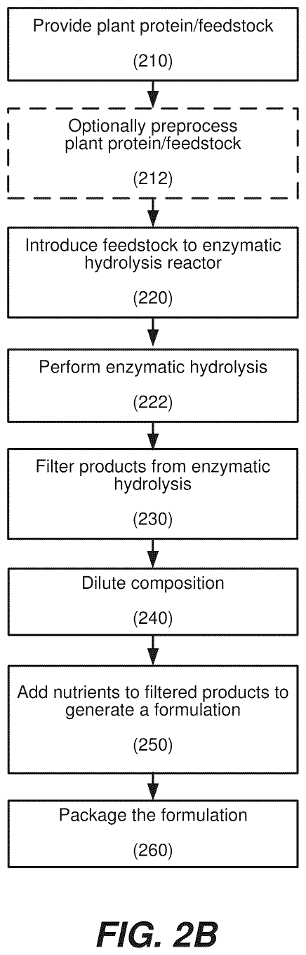

FIG. 2A provides a process flow diagram depicting operations of a method embodiment described herein. In an operation 210, a plant protein and/or feedstock is provided. Example plant sources of feedstock, including plant-based proteins, are described herein and may include but are not limited to carobs, peanuts, rice, soybean, Plukenetia volubilis, and tarwi. The raw plant-based feedstock may be processed (such as ground to a meal), to achieve a feedstock with a particular particle size and water content. In some embodiments, the plant-based feedstock is dried and then milled.

Yet other plant-based protein sources include but are not limited to plant material from the Fabaceae and/or Leguminosae family. Particular examples of plant-based protein sources include plant material from the Ceratonia genus, the Arachis genus, the Lupinus genus, the Glycine genus and the Pisum genus. For example, carob germ or carobs (Ceratonia siliqua) may be a suitable plant-based protein source. Peanuts (Arachis hypogaea) may also be a suitable plant-based protein source. Tarwi (Lupinus mutabilis) may also be a suitable plant-based protein source. Soybean (Glycine max) may also be a suitable plant-based protein source. Peas (Pisum sativum) may also be a suitable plant-based protein source. Other suitable genera that may provide a protein source include but are not limited to Astragalus, Acacia, Indigofera, Crotalaria, and Mimosa.

Some plant-based protein sources may be from the Euphorbiaceae family. An example genus from this family is the Plukenetia genus. Plukenetia volubilis, or Sacha inchi, is a perennial plant that is native to tropical South America. Plukenetia volubilis may also be a suitable plant-based protein source as it may have significant protein content as well as omega-3 fatty acids, omega-6 fatty acids, and omega-9 fatty acids. Other plant-based protein sources may be from the Poaceae family. One example genus from this family is the Oryza genus. For example, rice may be a suitable plant-based protein source.

In various embodiments, parts of a plant may be used as the plant-based protein source. Example sources include but are not limited to roots, stems, husks, leaves, and seeds. In certain embodiments, plant feedstock is used with little or no preparation other than harvesting and optionally storing and/or milling. In some embodiments, plant feedstock is subject to a post-harvest process such as high temperature drying, oil extraction, or similar process. In peanut sources, after oil extraction, the remaining dry “cake” is used as feedstock. In carob sources, the whole seed with the husk is dried and milled to form the feedstock. In lupine sources, the beans are dried and milled to form the feedstock.

In some embodiments, the plant-based protein source may have at least about 60% protein content by weight of the prepared feedstock (such as dry cake of peanut feedstock), or at least about 50% protein content by weight, or at least about 30% protein content by weight.

Turning back to FIG. 2A, an optional operation 212 can include pre-processing the feedstock (such as a feedstock powder. Pre-processing may be performed to eliminate polyphenols in vegetable flour because they inhibit the functioning of protease enzymes. Various types of pre-hydrolysis may be performed. Examples include mechanical agitation, addition of water or other liquid, chemical processing such as chemical extraction, sieving, etc. In one example, during pre-hydrolysis processing, polyphenols are extracted from the meal or other feedstock using, e.g., ethanol. In another instance, the feedstock can be sterilized.

Proteases may be mixed with the plant-based feedstock powder. The pH may also be adjusted to make the pH suitable for the enzyme used. In some embodiments, enzymes for conducting enzymatic hydrolysis are added to the feedstock during a preprocessing operation. In an operation 220, the feedstock is introduced to an enzymatic hydrolysis reactor.

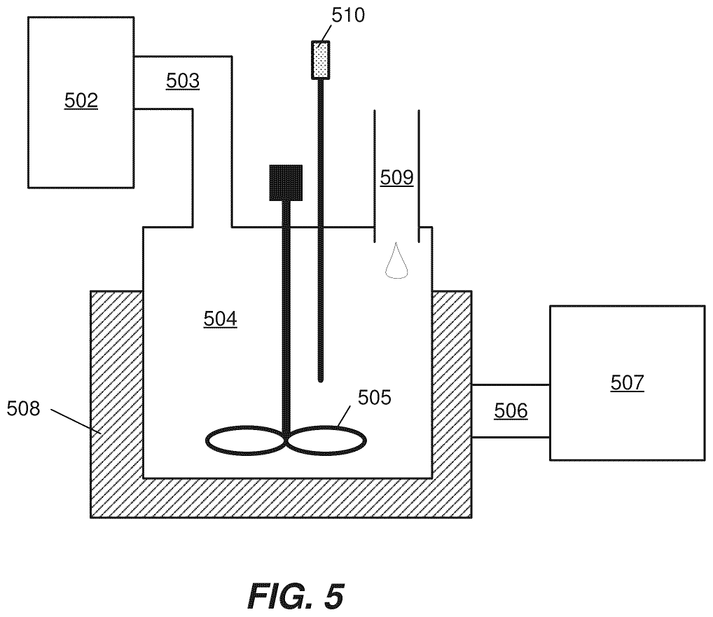

An example of a hydrolysis reactor is provided in FIG. 5. The enzymatic hydrolysis reactor may include a vessel 504 for containing and/or mixing various components, including processed feedstock and enzymes from a source 502 through inlet 503. In some embodiments, the enzymatic hydrolysis reactor includes a mixing or agitation mechanism such as propeller 505. The reactor also includes a pH probe 510 for measuring pH. pH and temperature are controlled in the vessel 504. The pH may be maintained at any useful pH range, depending on the type of protease employed in the reactor. In one instance, the pH range is between 7 and 9, or about 8.5. The pH can be controlled by including an inlet 509 for dripping acid or base fluids to regulate the pH. For example, 10 M of NaOH may be added to maintain a pH of about 8.5.

The temperature may be maintained at a temperature between about 55° C. and about 60° C. The temperature may be maintained by using heat sleeve 508, a heater, and/or a chiller. The enzymatic hydrolysis reactor can be configured to hydrolyze proteins in the feedstock to produce free amino acids and oligopeptides. Proteases can be added to the feedstock either before or after the feedstock is introduced to the reactor. Water may be added to the enzymes and/or feedstock, either before or after the feedstock is introduced to the reactor. Once, all the components are added to the reactor, the temperature and/or pressure of the reactor may be adjusted and, from there, enzymatic hydrolysis can proceed. In some embodiments, the plant-based feedstock includes enzymes, plant-based protein source as a powder, and water.

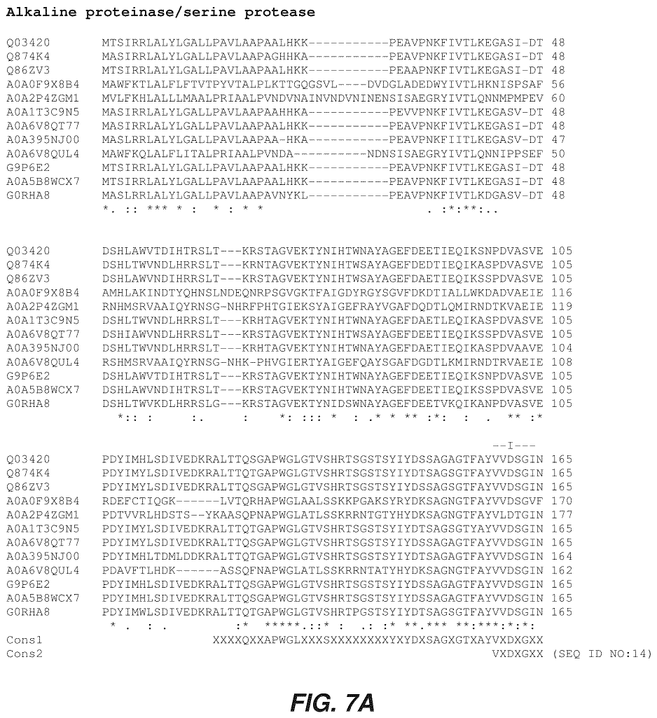

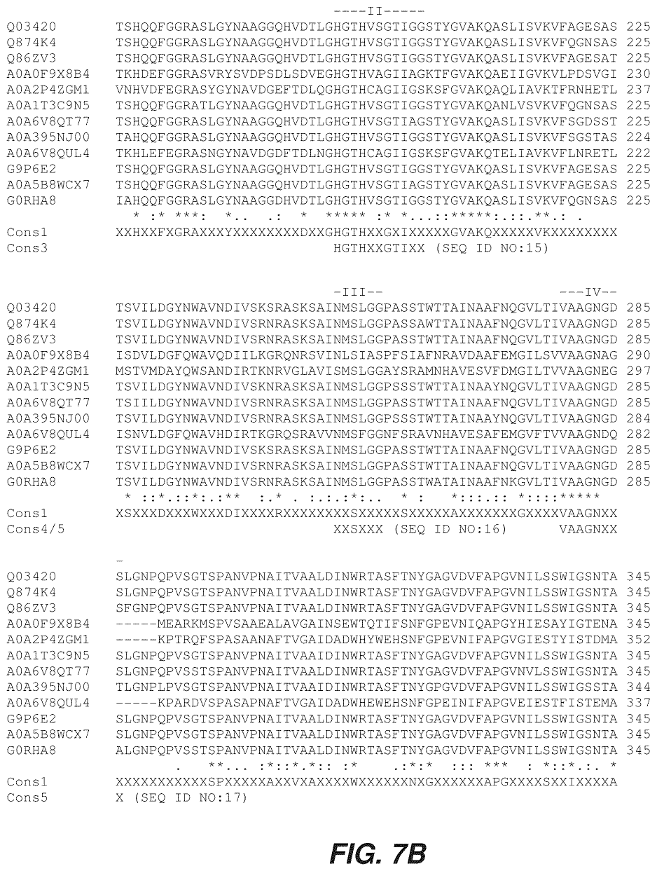

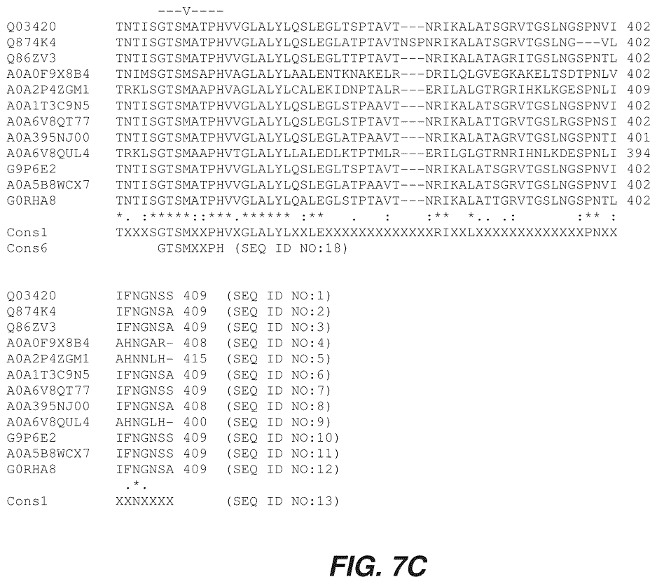

The type of enzyme used in enzymatic hydrolysis depends on the feedstock and the type of amino acid profile desired for the biostimulant composition. Enzymes are capable of breaking protein chains at a particular hydrolysis reaction rate. One enzyme that may be used is a protease that is a purified liquid enzymatic preparation. Some enzymes are widely available and widely used in the detergent production industry, the food industry, and in the textile industry. Examples of proteases that may be used for enzymatic hydrolysis include but are not limited to aspartic proteases, serine proteases, thiol proteases, metalloproteases, as well as any others described herein (e.g., in Tables 1-5 or FIGS. 7-13). Examples of aspartic proteases include but are not limited to pepsin, pepsin A, chymosin, and renin. Examples of serine proteases include but are not limited to trypsin, chymotrypsin, subtilisin novo, and alcalase. Examples of thiol proteases include but are not limited to pure papain and bromelain. Proteases may be derived from one or more of the following sources: ox, pig, calf, papaya, pineapple, Bacillus subtilis, Bacillus licheniformis, Aspergillus niger, Ananas comosus, and Aspergillus oryzae. Proteases may be provided as a mixture of various types of proteases. For example, a protease that is provided for enzymatic hydrolysis may include a mix of an aspartic protease, a metalloprotease, and a serine protease. Examples of protease mixtures include but are not limited to ProZyme™ available from PRN Pharmacal in Pensacola, FL; Panzyme™ available from Nutra BioGenesis in Park City, Utah Biozyme A™ available from G-Biosciences in St. Louis, MO, and Sanzyme available from Ciba Giegy of Switzerland.

Returning to FIG. 2A, in an operation 222, enzymatic hydrolysis is performed. During enzymatic hydrolysis, the following parameters can be monitored and controlled: substrate and enzyme concentration, reaction temperature, pH, and stirring speed. The reference substrate (e.g., vegetable flour) concentration of milled feedstock weight to water volume can be about 10% to 15% (w/v).

In one example, for enzymatic hydrolysis of carob germ, water is added to 300 grams of carob germ having a dry matter content of 55% to a final volume of 1 L, such that the resulting mixtures includes a concentration of protein content of 18% (w/v). The enzyme concentration during enzymatic hydrolysis may be about 0.1% to 0.2% (v/v) or about 0.15% (v/v). Enzymatic hydrolysis may be performed in the reactor at a temperature of about 45° C. to 55° C. or up to 60° C. In some embodiments, the mixture may be mixed for a duration of about 2 hours to 4 hours. The enzymatic hydrolysis may be performed at standard atmospheric pressure.

The pH of the enzymatic hydrolysis is determined by the pH suitable for the protease selected. Some enzymes are suitable for a pH of about 7 to 11, and some can have maximum activity at a pH of about 9. During enzymatic hydrolysis, concentrated NaOH may be added to maintain the pH in such way so as not to substantially increase the volume in the vessel.

Stirring speed may be adjusted throughout the enzymatic hydrolysis process depending on the texture of the hydrolysates. For example, when insoluble material solubilizes, stirring speed may be reduced to accommodate the newly soluble texture of the hydrolysates. Enzymatic hydrolysis may be performed until about 10% by weight or at least about 50% by weight of the amount of proteins in the feedstock is left unconverted to free amino acids, oligopeptides, and peptides.

After the hydrolysis process completes, the hydrolysis product may be optionally centrifuged. The centrifuged hydrolyzed mixture is removed from the reactor which may be performed by delivering via an outlet 506 of FIG. 5 to a filter 507. While proteinaceous material in the feedstock is broken down by proteases, other material in the feedstock is left wholly or partially unreacted. Examples of such unreacted materials include, micronutrients, macronutrients, phytohormones, and the secondary metabolites. Such unreacted material can include solid waste products, which can be separated by centrifugation, filtered, or otherwise removed and then discarded.

The hydrolysis product can be employed as a biostimulant composition or a biofungicidal composition. Alternatively, further operations can be conducted to further isolate, treat, or modify the hydrolysis product to provide a formulation. In some embodiments, after hydrolysis, hydrolyzing enzymes are inactivated by, e.g., a temperature shock. As seen in FIG. 2B, in an operation 230, the products from the enzymatic hydrolysis can be filtered. In some embodiments, two filtrations are carried out (coarse and fine). The first filtration eliminates solids, and the second eliminates further contaminants and solids, which are smaller in size. After filtration, in certain embodiments, the product is concentrated to a density of about 1 to 1.3 g/mL, such as about 1.18 g/mL. Finally, in some embodiments, the resulting product is pasteurized to eliminate microorganism contaminants.

In an operation 240, the biostimulant composition or the biofungicidal composition can be diluted to an amount, such as those described above. In some embodiments, water is added to the composition to achieve a water content of at least about 40% by volume.

In an operation 250, nutrients such as micronutrients and/or macronutrients are added to the filtered and diluted products to generate a biostimulant formulation. The micronutrients and macronutrients can be mixed with the products from the reactor to form a homogeneous mixture, which may prevent particles from sinking to the bottom of the liquid. Mixing may be performed using a paddle or other mechanical component, which may be automatically or manually controlled. Micronutrients include but are not limited to iron, manganese, boron, molybdenum, zinc, chlorine, sodium, and cobalt. One, two, three, or more of the above micronutrients may be added. The amount added may be such that they result in the concentration of each micronutrient including both added micronutrients and existing micronutrients from the plant-based protein source, in the biostimulant composition to be of about 1% to 15% by weight.

Macronutrients include nitrogen, phosphorous, potassium, calcium, sulfur, magnesium, carbon, oxygen, and hydrogen, which may also be added such that the resulting concentration of one or more of the macronutrients is about 1% to 15% by weight. In some embodiments, macronutrients are not added.

In an operation 260, the diluted composition can be packaged. As described above, the diluted composition may be packaged in liquid form into containers (e.g., bottles) of any of various sizes, such as 1 L bottles.

In some embodiments, the methods herein allow for enzymatic processing of feedstock that are generally deemed to be of low value or of low accessibility. For instance, some feedstocks that may be used have organic origin that have traditionally been considered directly as waste, or at most, are considered low added value materials. These different agro-industrial by-products have properties that give them great potential for application in the agricultural biotechnology industry. In another instance, these starting materials are not easily usable, as they are not accessible or available. For example, its high insolubility, mainly, makes its use difficult. However, enzyme technology, with extraction and/or modification processes, can convert these organic materials into new products with greater functionality, due to the concentration of active principles, and better application technological properties (increased solubility and decreased molecular size of its components).

The compositions and formulations described herein can be applied to crops or plants in various ways. Methods of using the composition and formulation can include treating a plant, such as by preparing any composition or a formulation herein; and delivering the composition or the formulation to a plant, a portion thereof, a plant material, or a soil in proximity to the plant. The composition or the formulation can be a biostimulant, a biofungicide, and/or a BCA. In one embodiment, the formulation includes the composition and another component, such as an added aqueous solvent (e.g., water). Such methods can be useful for, in some instances, in preventative treatment of plants or plant materials.

The compositions and formulations herein can be applied or delivered to a plant in any useful manner. Delivering can include any useful method, such as by providing the composition or formulation by way of fertigation delivery to an irrigation system (e.g., a drip irrigation system), or by way of foliar delivery directly to plants. In one embodiment, the composition or formulation can be applied by dissolving in an aqueous solvent prior to delivery to the soil, the plant, or the plant material (e.g., seed, seedling, bulb, etc.). In another embodiment, application can include pilling the seeds of a plant (e.g., using any coating described herein). In yet another embodiment, application can include immersion of the plant or the plant material in a bath containing a composition or formulation herein. Furthermore, application can include providing one or more additives (e.g., any herein) before, during, or after delivery of the composition or formulation to the soil, the plant, or the plant material. Other modes of delivery include broadcast application, liquid or dry in-furrow application, spraying, atomizing, vaporizing, misting, scattering, dusting, coating, watering, squirting, sprinkling, pouring, fumigating, and the like to the locus to be protected.

In particular embodiments, delivery can result in treating a plant. Such treating can include protecting the plant against a pathogenic organism, stimulating growth of the plant, or counteracting growth of a pathogenic organism in proximity to the plant. Use can include delivering an effective amount of the composition or formulation onto the soil and/or on the plants for such treatment. Such delivery can include drip irrigation (e.g., at or close to transplanting, with optional delivery at a later interval after 14-30 days), spraying (e.g., directed either in furrow during planting or to the soil surface after planting), soaking (e.g., soaking of soil; and/or soaking of plants or plant materials prior to planting), soil treatment (before or after sowing or transplanting), direct mixing with soil, direct application to seeds, and the like. Such treatment can provide, for instance, better root development, increased proliferation of secondary roots, increased seedling weight, and/or improved crop yields.

Delivery or treatment can include any useful regime. In one instance, delivery includes providing the composition or formulation at or close to transplanting. In another instance, delivery can include application at one or more stages of plant growth (e.g., germination, flowering, fruiting, etc.). Application can include one or more locations on the plant or soil (e.g., stem, leaves, roots, flowers, and/or fruit). Such application can include multiple applications to a single plant at different times (e.g., at or close to transplanting, with optional delivery at a later interval after 14-30 days).

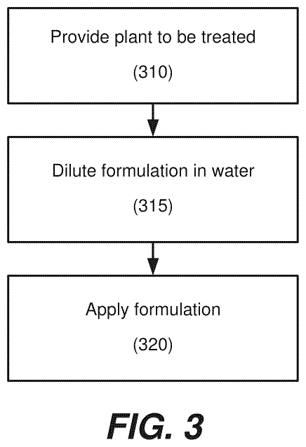

Prior to applying to crops, a biostimulant composition can be diluted. FIG. 3 provides a process flow diagram depicting operations that may be performed in accordance with certain embodiments. In operation 310, the plant to be treated is located or provided. The plant can be any one of a variety of crops, both ones having intensive short cycles and extensive long cycles. Examples include but are not limited to vegetables, industrial grains, berries, sugar cane, fruit trees, superfoods, and grapes. Biostimulants are not crop specific and can be useful for the vast majority of crops grown, including agricultural, medical and horticultural crops. They can be used in organic or conventional farming. Each plant type can utilize a different application regime of biostimulant, to maximize productivity.

In operation 315, a biostimulant is diluted to an amount such as those described above. In some embodiments, water is added to the biostimulant composition to achieve a water content of at least about 40% by volume.

In operation 320, the diluted biostimulant is applied to a target crop. When the diluted biostimulant is applied depends on the composition of the biostimulant, the amount of diluted biostimulant applied, and the time in the life cycle of the plant that can take advantage of the benefits of the biostimulant composition. Plants undergo various stages of life in their life cycles: seeds, sprouts or germination, seedlings, adult plants that undergo pre-flowering, flowering, pre-fruiting, and/or fruiting. Plants undergo reproduction and pollination, which may involve growth of flowers and/or fruits, prior to seed spreading. Some plants in different parts of their life cycles can use different amounts of a diluted biostimulant. Some plants in different parts of their life cycles can use different amounts of the same biostimulant. Biostimulant compositions can be applied to various parts of a plant, such as the seed, seedling, stem, leaves, branches, flowers, and fruit, and its surroundings, including the soil. The diluted biostimulant may be applied to a plant in a pot, or a plant grown by hydroponics, or a plant grown in an open field. Each of these types of plants may utilize different amounts of biostimulant.

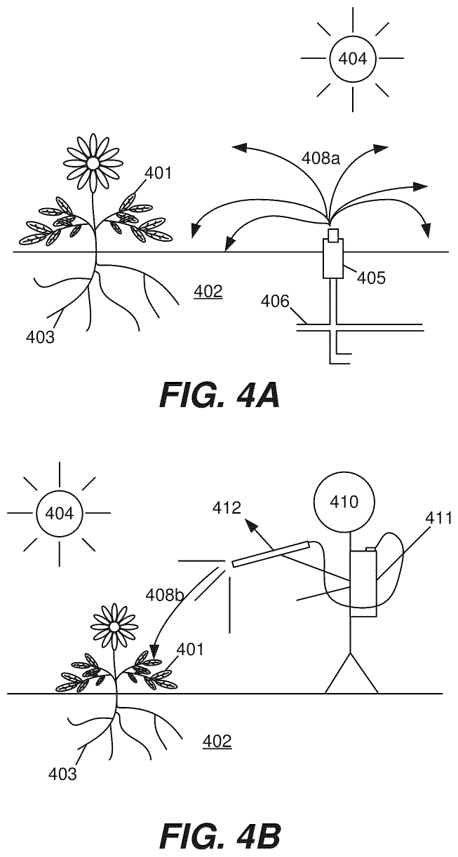

The location in which the diluted biostimulant is applied may also vary from plant to plant. For example, in some embodiments, irrigation systems are used, such as shown in the example in FIG. 4A, which includes a schematic diagram of a plant 401 having roots 403 in soil 402 under a light source 404 (in this case, the sun). Also provided is an irrigation system having piping 406 and a delivery spout 405, whereby the trajectory 408a of a diluted biostimulant may be used to apply the diluted biostimulant via irrigation.

In some embodiments, diluted biostimulants are applied directly to a plant, such as to the leaves or the foliage of a plant and may be manually applied by a person. An example is provided in FIG. 4B, which is a schematic diagram of a plant 401 having roots 403 in soil 402 under a light source 404, whereby the trajectory 408b of a diluted biostimulant is delivered or sprayed via a mister 412 handled by a human 410 from a container 411 of biostimulant. Where the diluted biostimulant is applied depends on environmental variables as well.

Any of the discussions herein regarding the diluted biostimulant could be used to form and deliver a diluted biofungicide. For instance, the timing of application and amount of a dilute biofungicide could depend on the plant life cycle, environmental variables, and the like.

The compositions or formulations herein can be employed in any useful plant, portion thereof (e.g., roots, tubers, stems, flowers, and/or leaves), plant material (e.g., seed, seedlings, bulbs, etc.), or soil for growing such plants. Non-limiting plants include crop plants, turfs, ornamentals, agronomic row or other field crops, plants prone to fungal contamination (e.g., tomatoes, bell peppers, soy, and the like), and plants prone to new pathogenic fungi (e.g., rice, wheat, and the like). Yet other plants include agricultural plant species, including coffee, cucumber, tomato, pepper, and radish.

In some embodiments, the compositions and formulations herein can be used against pathogens, including pathogenic fungi. Non-limiting pathogens include Botryosphaeria (e.g., B. dothidea), Botrytis (e.g., B. cinerea), Clarireedia (e.g., C. homoeocarpa), Colletotrichum (e.g., C. coccodes), Fusarium (e.g., F. oxysporum), Macrophomina (e.g., M. phaseolina), Phytophthora (e.g., P. cactorum, P. cinnamomi, P. citricola, P. citrophthora, P. cryptogea, P. drechsleri, P. infestans, and/or P. nicotianae), Pythium (e.g., P. aphanidermatum, P. irregulare, P. spiculum, and/or P. ultimum), Rhizoctonia (e.g., R. solani), Rosellinia (e.g., R. necatrix), Sclerotinia (e.g., S. homoeocarpa, S. sclerotiorum, and/or S. solfsii), Sclerotium (e.g., S. rolfsii), Thielaviopsis (e.g., T. basicola) species, Verticillium (e.g., V. dahliae), as well as combinations thereof.

Proteases from Trichoderma and Formulations Thereof

One or more proteases may be employed to treat a feedstock. In some embodiments, the protease(s) can be from at least one particular Trichoderma species. In other embodiments, the protease(s) can be from at least two particular Trichoderma species. In some cases, the species is selected from Trichoderma parareesei, Trichoderma virens, Trichoderma atroviride, and Trichoderma asperellum. The protease can be provided as isolated proteins (e.g., isolated proteases) or as isolates, which can include spores, mycelium, or both.

The protease(s) can be from a combination of multiple species, such as two, three, four, or more species. In one instance, the compositions, formulations, and methods can include one or more proteases from T. parareesei (e.g., strain T6) and T. virens (e.g., strain T59). One or more proteases can be provided from other Trichoderma species. For instance, the compositions, formulations, and methods can further include one or more proteases from T. atroviride (e.g., strain T11) and/or T. asperellum (e.g., strain T25). In other embodiments, the compositions, formulations, and methods can include a combination of one or more proteases from multiple strains of the same species, such as two, three, four, or more strains. In yet other embodiments, the compositions, formulations, and methods can include a combination of one or more proteases from multiple strains of different species, in which at least two strains or two species are different. Further Trichoderma species are described herein.

The relative amount of each protease from each species or strain can provide an effective amount to provide a particular effect. Such an effective amount can be determined by the amount that can provide a particular profile (e.g., amino acid profile) within the hydrolysis product.

A protease (e.g., EC 3.4.11; EC 3.4.13-19; EC 3.4.21-24; and EC 3.4.99) can include exopeptidases, endopeptidases, alkaline proteases, acid proteases, serine proteases (EC 3.4.21), serine carboxy proteases (EC 3.4.16), cysteine proteases (EC 3.4.22), aspartic proteases (EC 3.4.23), metalloproteases I (EC 3.4.24), metallocarboxy proteases (EC 3.4.17), aspergillopepsin (EC 3.4.23.18), trypsin (EC 3.4.21.4), saccharopepsin (EC 3.4.23.25), cerevisin (EC 3.4.21.48), and carboxypeptidase B (EC 3.4.17.2).

Non-limiting serine proteases include trypsin-like protease, chymotrypsin-like protease, subtilisin-like protease, alkaline serine protease, serine carboxypeptidase, dipeptidyl peptidase, dipeptidase E, sedolisin, and others. In particular embodiments, use of serine proteases includes providing a pH of around 6-10, e.g., about 8-9.

In one example, the alkaline proteinases and serine proteases are provided as SEQ ID NOs:1-12 (FIG. 7A-7C). In some embodiments, the protease includes a polypeptide sequence having at least 90% sequence identity to any one of SEQ ID NOs:1-12.

In other examples, the serine protease includes a polypeptide sequence having at least 90% sequence identity to SEQ ID NO:13, in which each X is any amino acid provided in an aligned reference sequence (e.g., aligned reference sequences SEQ ID NOs:1-12 for the consensus sequence of SEQ ID NO:13), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix).

In yet other examples, the serine protease includes a polypeptide sequence having at least 90% sequence identity to any one of SEQ ID NOs:14-18. In some embodiments, the serine protease includes a polypeptide sequence having at least 90% sequence identity to each of SEQ ID NOs:14-18, in which each of SEQ ID NOs:14-18 is optimally aligned when the polypeptide sequence is used as the reference sequence.

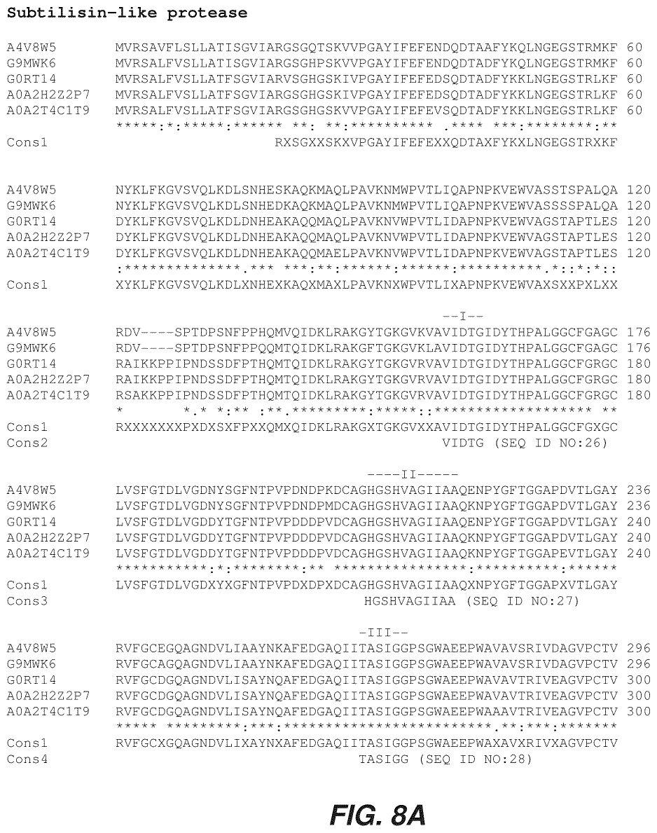

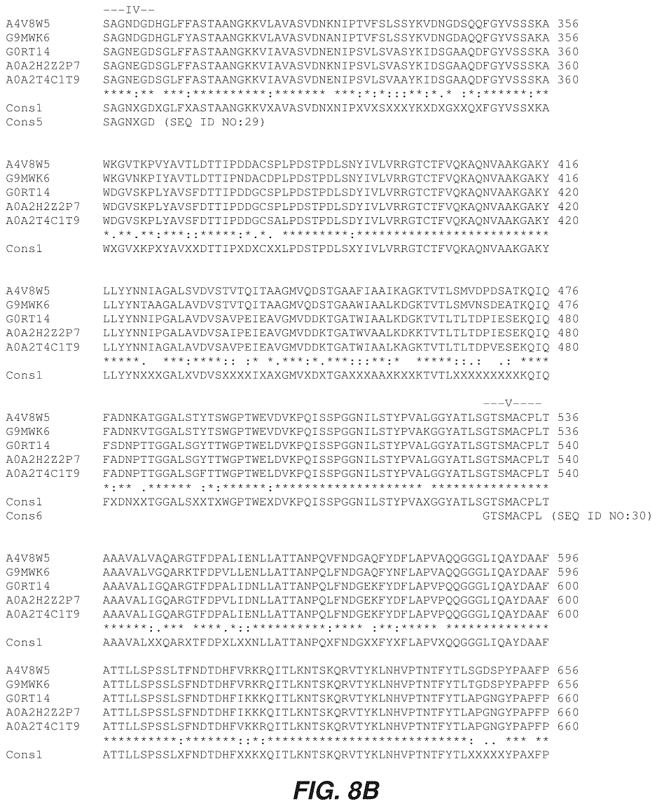

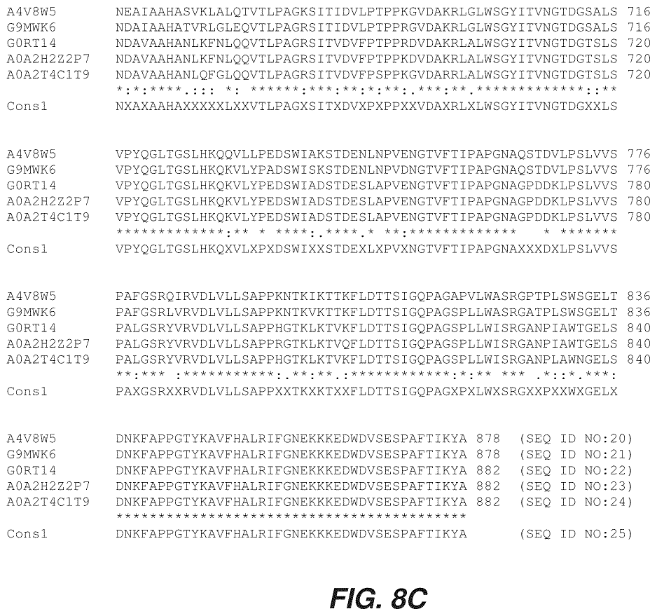

In one example, the subtilisin-like proteases are provided as SEQ ID NOs:20-24 (FIG. 8A-SC). In some embodiments, the protease includes a polypeptide sequence having at least 90% sequence identity to any one of SEQ ID NOs:20-24.

In other examples, the subtilisin-like protease includes a polypeptide sequence having at least 90% sequence identity to SEQ ID NO:25, in which each X is any amino acid provided in an aligned reference sequence (e.g., aligned reference sequences SEQ ID NOs:20-24 for the consensus sequence of SEQ ID NO:25), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix).

In yet other examples, the subtilisin-like protease includes a polypeptide sequence having at least 90% sequence identity to any one of SEQ ID NOs:26-30. In some embodiments, the protease includes a polypeptide sequence having at least 90% sequence identity to each of SEQ ID NOs:26-30, in which each of SEQ ID NOs:26-30 is optimally aligned when the polypeptide sequence is used as the reference sequence.

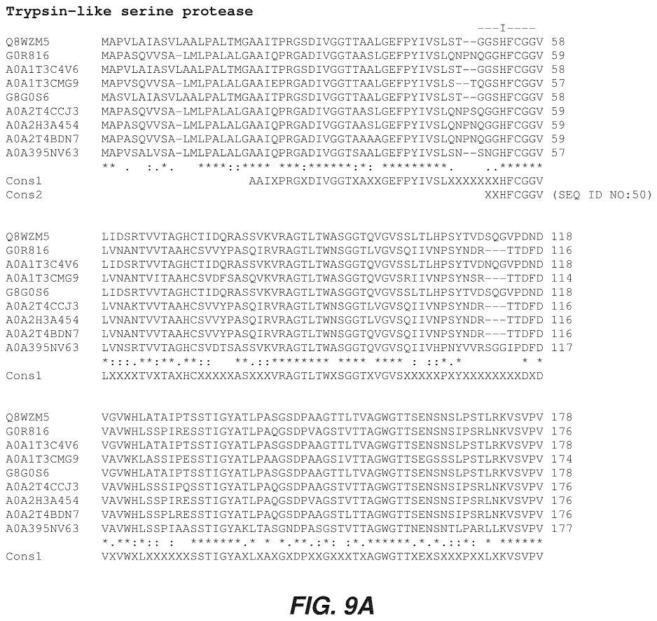

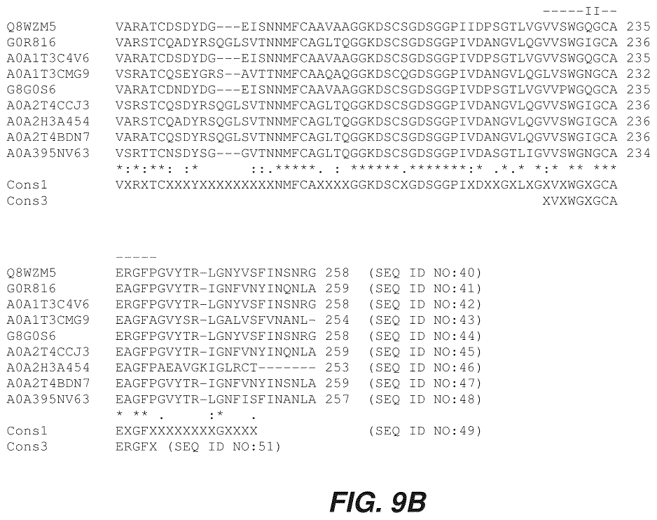

In one example, the trypsin-like proteases are provided as SEQ ID NOs:40-48 (FIG. 9A-9B). In some embodiments, the protease includes a polypeptide sequence having at least 90% sequence identity to any one of SEQ ID NOs:40-48.

In other examples, the trypsin-like protease includes a polypeptide sequence having at least 90% sequence identity to SEQ ID NO:49, in which each X is any amino acid provided in an aligned reference sequence (e.g., aligned reference sequences SEQ ID NOs:40-48 for the consensus sequence of SEQ ID NO:49), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix).

In yet other examples, the trypsin-like protease includes a polypeptide sequence having at least 90% sequence identity to any one of SEQ ID NOs:50-51. In some embodiments, the protease includes a polypeptide sequence having at least 90% sequence identity to each of SEQ ID NOs:50-51, in which each of SEQ ID NOs:50-51 is optimally aligned when the polypeptide sequence is used as the reference sequence.

Non-limiting acid proteases include aspartyl protease/aspartic proteases, glutamic proteases, glutamine proteases, eqolisins, and others. In particular embodiments, the use of acid proteases includes providing a pH of around 2-5, e.g., about 3-4.

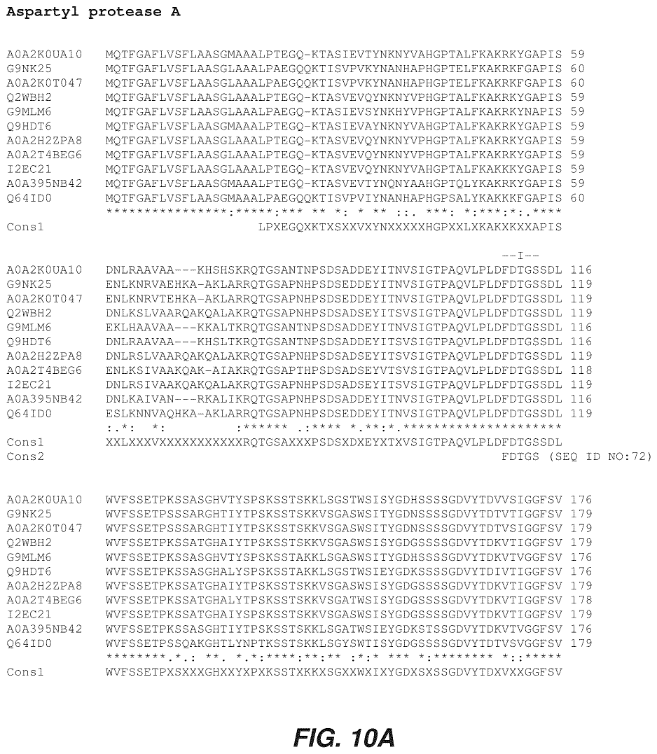

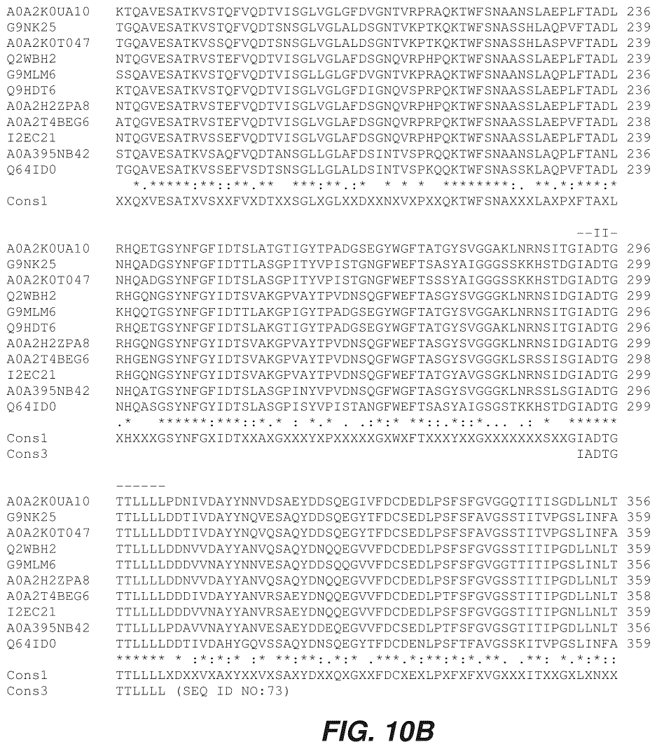

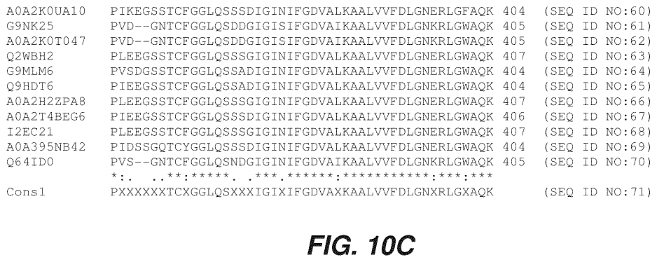

In one example, the aspartyl proteases are provided as SEQ ID NOs:60-70 (FIG. 10A-10C). In some embodiments, the protease includes a polypeptide sequence having at least 90% sequence identity to any one of SEQ ID NOs:60-70.

In other examples, the aspartyl protease includes a polypeptide sequence having at least 90% sequence identity to SEQ ID NO:71, in which each X is any amino acid provided in an aligned reference sequence (e.g., aligned reference sequences SEQ ID NOs:60-70 for the consensus sequence of SEQ ID NO:71), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix).

In yet other examples, the aspartyl protease includes a polypeptide sequence having at least 90% sequence identity to any one of SEQ ID NOs:72-73. In some embodiments, the protease includes a polypeptide sequence having at least 90% sequence identity to each of SEQ ID NOs:72-73, in which each of SEQ ID NOs:72-73 is optimally aligned when the polypeptide sequence is used as the reference sequence.

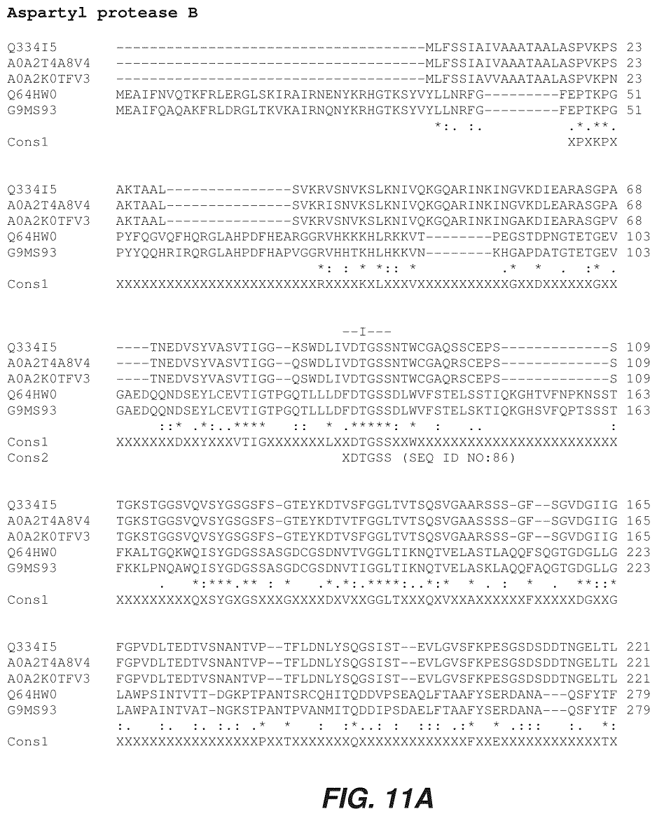

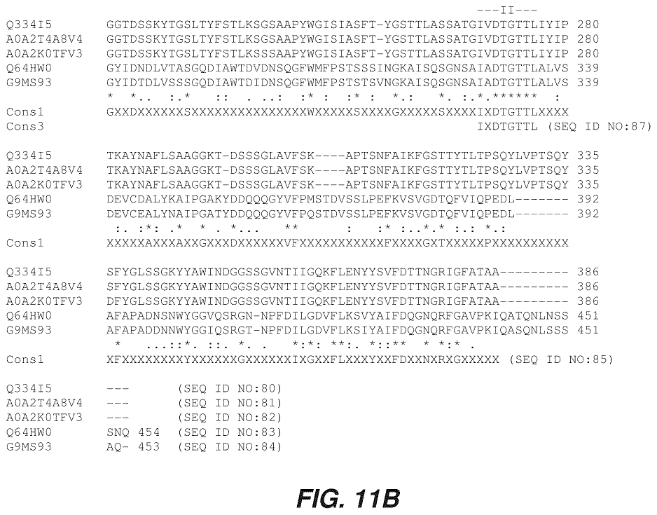

In another example, the aspartyl proteases are provided as SEQ ID NOs:80-84 (FIG. 11A-11B). In some embodiments, the protease includes a polypeptide sequence having at least 90% sequence identity to any one of SEQ ID NOs:80-84.

In other examples, the aspartyl protease includes a polypeptide sequence having at least 90% sequence identity to SEQ ID NO:85, in which each X is any amino acid provided in an aligned reference sequence (e.g., aligned reference sequences SEQ ID NOs:80-84 for the consensus sequence of SEQ ID NO:85), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix).

In yet other examples, the aspartyl protease includes a polypeptide sequence having at least 90% sequence identity to any one of SEQ ID NOs:86-87. In some embodiments, the protease includes a polypeptide sequence having at least 90% sequence identity to each of SEQ ID NOs:86-87, in which each of SEQ ID NOs:86-87 is optimally aligned when the polypeptide sequence is used as the reference sequence.

Non-limiting metalloproteases or metallopeptidases include carboxypeptidase (e.g., carboxypeptidase A or carboxypeptidase B), glutamate carboxypeptidase, methionine-aminopeptidase, aminopeptidase (e.g., aminopeptidase Y), protease M35 (deuterolysin), protease M36 (fungalysin), peptidase M6 (InhA-like protease), and others. In particular embodiments, the use of metalloproteases includes providing a metal (e.g., a divalent metal, such as zinc or cobalt). Non-limiting cysteine proteases include aspartyl endopeptidase (legumain).

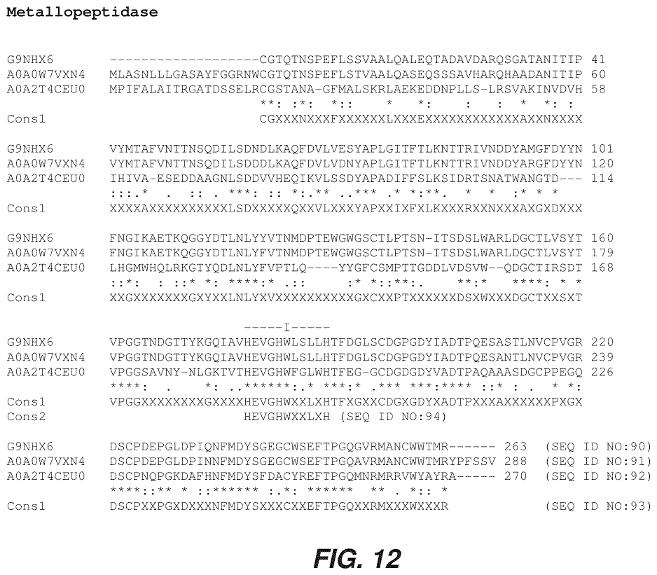

In one example, the metallopeptidases are provided as SEQ ID NOs:90-92 (FIG. 12). In some embodiments, the protease includes a polypeptide sequence having at least 90% sequence identity to any one of SEQ ID NOs:90-92.

In other examples, the metallopeptidase includes a polypeptide sequence having at least 90% sequence identity to SEQ ID NO:93, in which each X is any amino acid provided in an aligned reference sequence (e.g., aligned reference sequences SEQ ID NOs:90-92 for the consensus sequence of SEQ ID NO:93), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring=<0.5 and >0 in the Gonnet PAM 250 matrix). In yet other examples, the metallopeptidase includes a polypeptide sequence having at least 90% sequence identity to SEQ ID NO:94.

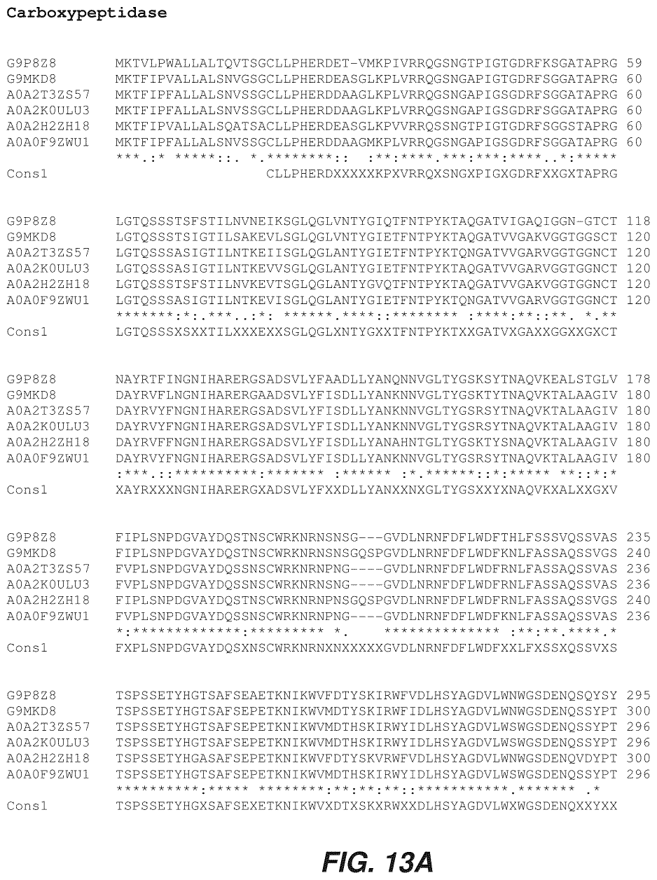

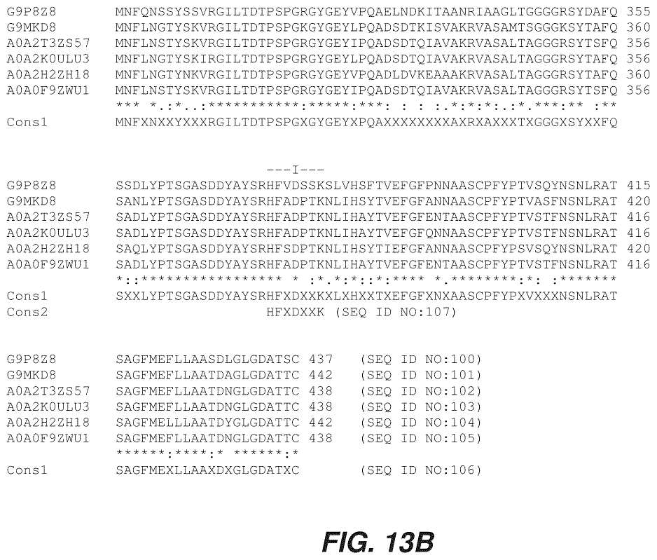

In another example, the carboxypeptidases are provided as SEQ ID NOs:100-105 (FIG. 13A-13B). In some embodiments, the protease includes a polypeptide sequence having at least 90% sequence identity to any one of SEQ ID NOs:100-105.

In other examples, the carboxypeptidase includes a polypeptide sequence having at least 90% sequence identity to SEQ ID NO:106, in which each X is any amino acid provided in an aligned reference sequence (e.g., aligned reference sequences SEQ ID NOs:100-105 for the consensus sequence of SEQ ID NO:106), a conservative amino acid substitution for any amino acid provided in an aligned reference sequence, an amino acid having strongly similar properties for any amino acid provided in an aligned reference sequence (e.g., equivalent to scoring >0.5 in the Gonnet PAM 250 matrix), or an amino acid having weakly similar properties for any amino acid provided in an aligned reference sequence (e.g., equivalent to scoring <0.5 and >0 in the Gonnet PAM 250 matrix). In yet other examples, the carboxypeptidase includes a polypeptide sequence having at least 90% sequence identity to SEQ ID NO: 107.

Further proteases are provided below, including serine proteases (Table 1), aspartate proteases (Table 2), metalloproteases (Table 3), glutamate/glutamine proteases (Table 4), and cysteine proteases (Table 5) from a Trichoderma species.

TABLE 1
Non-limiting serine proteases from Trichoderma

| UniProtKB | | | Length |
|---|---|---|---|
| No. | Protein names | Organism | [aa] |
| G0R816 | Trypsin-like serine protease | T. reesei | 259 |
| G0RW36 | Predicted protein | T. reesei | 533 |
| G0R938 | Predicted protein | T. reesei | 286 |
| G0RXF1 | Predicted protein | T. reesei | 637 |
| G0RUJ7 | Predicted protein | T. reesei | 555 |
| G0RXE9 | Predicted protein | T. reesei | 612 |
| G0RT14 | Predicted protein | T. reesei | 882 |
| G0REN2 | Serine protease | T. reesei | 388 |
| A0A2H3A454 | Trypsin-like protease | T. parareesei | 253 |
| A0A2H2ZBP2 | Carboxypeptidase (EC 3.4.16.-) | T. parareesei | 532 |
| A0A2H2ZH18 | Carboxypeptidase A | T. parareesei | 442 |
| A0A2H2Z7U4 | Carboxypeptidase (EC 3.4.16.-) | T. parareesei | 558 |
| A0A2H2YVL3 | Peptidase S53 domain-containing protein | T. parareesei | 481 |
| A0A2H2ZJE3 | Tripeptide peptidase | T. parareesei | 637 |
| A0A2H2ZDJ6 | Subtilisin like protease (SUB3) | T. parareesei | 433 |
| A0A2H2Z2P7 | Subtilisin-like protease PPRC1 | T. parareesei | 882 |
| A0A2T4AHU5 | Peptidase S1 domain-containing protein | T. harzianum | 254 |
| A0A2T4AS89 | Peptidase S1 domain-containing protein | T. harzianum | 258 |
| A0A2T4A2L2 | Carboxypeptidase (EC 3.4.16.-) | T. harzianum | 575 |
| A0A2T4API9 | Carboxypeptidase (EC 3.4.16.-) | T. harzianum | 548 |
| A0A2T4AGG8 | Uncharacterized protein | T. harzianum | 621 |
| A0A2T3ZS57 | Peptidase_M14 domain-containing protein | T. harzianum | 438 |
| A0A2T4ABC6 | Carboxypeptidase (EC 3.4.16.-) | T. harzianum | 436 |
| A0A2T3ZUL6 | Uncharacterized protein (Fragment) | T. harzianum | 235 |
| A0A2T4ASE5 | Peptidase S53 domain-containing protein | T. harzianum | 711 |
| A0A2T3ZW21 | Peptidase S53 domain-containing protein | T. harzianum | 618 |
| A0A2T3ZVF2 | Peptidase S53 domain-containing protein | T. harzianum | 621 |
| A0A2T3ZW26 | Peptidase S53 domain-containing protein | T. harzianum | 637 |
| A0A2T4AL11 | Peptidase S53 domain-containing protein | T. harzianum | 649 |
| A0A2T4AL37 | Peptidase S53 domain-containing protein | T. harzianum | 626 |
| A0A2T4ACG8 | Peptidase S53 domain-containing protein | T. harzianum | 578 |
| A0A2T4AKI5 | Peptidase S53 domain-containing protein | T. harzianum | 631 |
| A0A2T4AL09 | Peptidase S53 domain-containing protein | T. harzianum | 587 |
| A0A2T4ACZ2 | Peptidase_S8 domain-containing protein | T. harzianum | 433 |
| A0A2T4A8Y8 | Uncharacterized protein | T. harzianum | 918 |
| A0A2T3ZSB7 | Uncharacterized protein | T. harzianum | 920 |
| A0A2T4AN88 | Uncharacterized protein | T. harzianum | 878 |
| G9MMR2 | Peptidase S1 domain-containing protein | T. virens | 254 |
| G9MZZ8 | Peptidase S1 domain-containing protein | T. virens | 258 |
| G9NCM3 | Carboxypeptidase (EC 3.4.16.-) | T. virens | 548 |
| G9NBT0 | Peptidase S53 domain-containing protein | T. virens | 637 |
| G9MY16 | Peptidase S53 domain-containing protein | T. virens | 685 |
| G9MSU4 | Peptidase S53 domain-containing protein | T. virens | 706 |
| G9MX31 | Peptidase S53 domain-containing protein | T. virens | 624 |
| G9NBT2 | Peptidase S53 domain-containing protein | T. virens | 612 |
| G9MJL0 | Peptidase S53 domain-containing protein | T. virens | 556 |
| G9MFA6 | Uncharacterized protein | T. virens | 407 |
| G9MIQ9 | Peptidase S8 domain-containing protein | T. virens | 437 |
| G9N8A5 | Uncharacterized protein | T. virens | 391 |
| G9NA09 | Uncharacterized protein | T. virens | 402 |
| G9MZX8 | Uncharacterized protein | T. virens | 409 |
| G9MWK6 | Uncharacterized protein | T. virens | 878 |
| G9MK68 | Uncharacterized protein | T. virens | 892 |
| G9NM07 | Peptidase S1 domain-containing protein | T. atroviride | 255 |
| G9NZ06 | Peptidase_M43 domain-containing protein | T. atroviride | 307 |
| | (Fragment) | | |
| G9NEQ8 | Uncharacterized protein | T. atroviride | 533 |
| G9NKD1 | Uncharacterized protein | T. atroviride | 286 |
| G9NYD6 | Peptidase S53 domain-containing protein | T. atroviride | 637 |
| G9NTV7 | Peptidase S53 domain-containing protein | T. atroviride | 685 |
| G9NE12 | Peptidase S53 domain-containing protein | T. atroviride | 679 |
| G9NRV9 | Peptidase S53 domain-containing protein | T. atroviride | 665 |
| G9NQ23 | Peptidase S53 domain-containing protein | T. atroviride | 641 |
| | (Fragment) | | |
| G9NPW8 | Peptidase S53 domain-containing protein | T. atroviride | 704 |
| | (Fragment) | | |
| G9NEV6 | Peptidase S53 domain-containing protein | T. atroviride | 637 |
| G9NEV8 | Peptidase S53 domain-containing protein | T. atroviride | 591 |
| G9NHT4 | Peptidase S53 domain-containing protein | T. atroviride | 609 |
| G9PC25 | Peptidase S53 domain-containing protein | T. atroviride | 631 |
| G9PBS5 | Peptidase_S8 domain-containing protein | T. atroviride | 439 |
| G9P6E2 | Uncharacterized protein | T. atroviride | 409 |
| G9P7N9 | Uncharacterized protein | T. atroviride | 884 |
| G9P8R5 | Uncharacterized protein | T. atroviride | 913 |
| G9PCI8 | Uncharacterized protein | T. atroviride | 887 |
| A0A2T3ZHR2 | Carboxypeptidase (EC 3.4.16.-) | T. asperellum | 519 |
| A0A2T3YRE2 | Carboxypeptidase (EC 3.4.16.-) | T. asperellum | 621 |
| A0A2T3ZQ59 | Carboxypeptidase (EC 3.4.16.-) | T. asperellum | 550 |
| A0A2T3Z5X7 | Uncharacterized protein | T. asperellum | 532 |
| A0A2T3ZG11 | Uncharacterized protein | T. asperellum | 286 |
| A0A2T3Z8C1 | Peptidase S53 domain-containing protein | T. asperellum | 589 |
| A0A2T3Z3V9 | Peptidase S53 domain-containing protein | T. asperellum | 609 |
| A0A2T3Z657 | Peptidase S53 domain-containing protein | T. asperellum | 614 |
| A0A2T3Z670 | Peptidase S53 domain-containing protein | T. asperellum | 637 |
| A0A2T3YXL7 | Peptidase S53 domain-containing protein | T. asperellum | 703 |
| A0A2T3ZAM8 | Peptidase S53 domain-containing protein | T. asperellum | 685 |
| A0A2T3Z8Q8 | Peptidase S53 domain-containing protein | T. asperellum | 631 |
| A0A2T3Z616 | Peptidase S53 domain-containing protein | T. asperellum | 653 |
| A0A2T3Z7B8 | Peptidase_S8 domain-containing protein | T. asperellum | 438 |
| A0A2T3ZMF3 | Uncharacterized protein | T. asperellum | 882 |
| A0A2T3ZJC6 | Uncharacterized protein | T. asperellum | 924 |
| A0A2T3ZPH2 | Uncharacterized protein | T. asperellum | 867 |
| A0A2T3Z886 | Uncharacterized protein | T. asperellum | 909 |

TABLE 2
Non-limiting aspartate proteases from Trichoderma

| UniProtKB | | | Length |
|---|---|---|---|
| No. | Protein names | Organism | [aa] |
| G0R6X8 | Predicted protein | T. reesei | 477 |
| G0RSP8 | Predicted protein | T. reesei | 426 |
| G0RGD6 | Predicted protein | T. reesei | 451 |
| G0RN40 | Predicted protein | T. reesei | 646 |
| G0RVH9 | Predicted protein | T. reesei | 372 |
| G0RFV3 | Predicted protein | T. reesei | 417 |
| G0RJF6 | Predicted protein | T. reesei | 419 |
| G0RKE2 | Predicted protein | T. reesei | 488 |
| G0R9K1 | Predicted protein | T. reesei | 399 |
| G0RIW3 | Predicted protein | T. reesei | 395 |
| G0R8T0 | Aspartate protease | T. reesei | 407 |
| G0RTY7 | Predicted protein | T. reesei | 430 |
| G0RS55 | Predicted protein | T. reesei | 427 |
| G0RG34 | Aspartic peptidase A1 | T. reesei | 452 |
| G0RIW3 | Predicted protein | T. reesei | 395 |
| A0A2H2Z6N9 | Aspartyl protease | T. parareesei | 399 |
| A0A2H3ABB1 | Aspartictype endopeptidase ctsD | T. parareesei | 516 |
| A0A2H2ZEG7 | Aspartyl protease | T. parareesei | 477 |
| A0A2H2ZQT8 | Aspartyl protease | T. parareesei | 426 |
| A0A2H2ZJJ0 | Aspartyl protease | T. parareesei | 488 |
| A0A2H2YXC2 | Aspartyl protease | T. parareesei | 372 |
| A0A2H2ZI61 | Aspartyl protease | T. parareesei | 436 |
| A0A2H2ZRC6 | Aspartyl protease | T. parareesei | 535 |
| A0A2T4A7T9 | Peptidase A1 domain-containing protein | T. harzianum | 515 |
| A0A2T3ZZM6 | Peptidase A1 domain-containing protein | T. harzianum | 504 |
| A0A2T3ZY81 | Peptidase A1 domain-containing protein | T. harzianum | 664 |
| A0A2T4A8X8 | Peptidase A1 domain-containing protein | T. harzianum | 383 |
| A0A2T3ZYZ2 | Peptidase A1 domain-containing protein | T. harzianum | 459 |
| A0A2T4APY8 | Peptidase A1 domain-containing protein | T. harzianum | 418 |
| A0A2T4AJK8 | Peptidase A1 domain-containing protein | T. harzianum | 473 |
| A0A2T4A8V4 | Peptidase A1 domain-containing protein | T. harzianum | 386 |
| A0A2T3ZWM1 | Peptidase A1 domain-containing protein | T. harzianum | 489 |
| A0A2T3ZUL1 | Peptidase A1 domain-containing protein | T. harzianum | 355 |
| A0A2T4A4V7 | Peptidase A1 domain-containing protein | T. harzianum | 372 |
| A0A2T4AP91 | Peptidase A1 domain-containing protein | T. harzianum | 411 |
| A0A2T3ZX38 | Peptidase A1 domain-containing protein | T. harzianum | 621 |
| A0A2T4ATY2 | Peptidase A1 domain-containing protein | T. harzianum | 530 |
| G9NC88 | Peptidase A1 domain-containing protein | T. virens | 417 |
| G9N404 | Peptidase A1 domain-containing protein | T. virens | 426 |
| G9N310 | Peptidase A1 domain-containing protein | T. virens | 457 |
| G9MR65 | Peptidase A1 domain-containing protein | T. virens | 529 |
| G9MXZ4 | Peptidase A1 domain-containing protein | T. virens | 372 |
| | (Fragment) | | |
| G9MS88 | Peptidase A1 domain-containing protein | T. virens | 536 |
| G9MSK5 | Peptidase A1 domain-containing protein | T. virens | 416 |
| G9NCX5 | Peptidase A1 domain-containing protein | T. virens | 410 |
| G9MHD2 | Peptidase A1 domain-containing protein | T. virens | 546 |
| | (Fragment) | | |
| G9MWZ0 | Peptidase A1 domain-containing protein | T. virens | 332 |
| G9N2C5 | Peptidase A1 domain-containing protein | T. virens | 488 |
| G9N3H5 | Peptidase A1 domain-containing protein | T. virens | 470 |
| G9MUE5 | Peptidase A1 domain-containing protein | T. virens | 400 |
| G9N023 | Peptidase A1 domain-containing protein | T. virens | 389 |
| G9MNJ2 | Peptidase A1 domain-containing protein | T. virens | 395 |
| G9MLM6 | Peptidase A1 domain-containing protein | T. virens | 404 |
| G9P711 | Peptidase A1 domain-containing protein | T. atroviride | 418 |
| G9NPY9 | Peptidase A1 domain-containing protein | T. atroviride | 513 |
| G9NU53 | Peptidase A1 domain-containing protein | T. atroviride | 437 |
| G9P8C3 | Peptidase A1 domain-containing protein | T. atroviride | 549 |
| | (Fragment) | | |
| G9PC71 | Peptidase A1 domain-containing protein | T. atroviride | 447 |
| G9NTT8 | Peptidase A1 domain-containing protein | T. atroviride | 372 |
| G9P5R4 | Peptidase A1 domain-containing protein | T. atroviride | 408 |
| G9NLJ7 | Peptidase A1 domain-containing protein | T. atroviride | 355 |
| G9PAL4 | Peptidase A1 domain-containing protein | T. atroviride | 488 |
| G9NUM7 | Peptidase A1 domain-containing protein | T. atroviride | 480 |
| G9NQ54 | Peptidase A1 domain-containing protein | T. atroviride | 401 |
| G9P228 | Peptidase A1 domain-containing protein | T. atroviride | 389 |
| G9NK25 | Peptidase A1 domain-containing protein | T. atroviride | 405 |
| A0A2T3YQS2 | Peptidase A1 domain-containing protein | T. asperellum | 391 |
| A0A2T3YXT1 | Peptidase A1 domain-containing protein | T. asperellum | 401 |
| A0A2T3ZJ75 | Peptidase A1 domain-containing protein | T. asperellum | 387 |
| A0A2T3ZFP3 | Peptidase A1 domain-containing protein | T. asperellum | 405 |
| A0A2T3Z090 | Peptidase A1 domain-containing protein | T. asperellum | 355 |
| A0A2T3Z9G1 | Peptidase A1 domain-containing protein | T. asperellum | 475 |
| A0A2T3YYD7 | Peptidase A1 domain-containing protein | T. asperellum | 353 |
| A0A2T3ZNE0 | Peptidase A1 domain-containing protein | T. asperellum | 416 |
| A0A2T3YZS4 | Peptidase A1 domain-containing protein | T. asperellum | 613 |
| A0A2T3YYV0 | Peptidase A1 domain-containing protein | T. asperellum | 489 |
| A0A2T3ZMT8 | Peptidase A1 domain-containing protein | T. asperellum | 424 |
| A0A2T3YXK7 | Peptidase A1 domain-containing protein | T. asperellum | 513 |
| A0A2T3Z8Y9 | Peptidase A1 domain-containing protein | T. asperellum | 455 |
| A0A2T3ZAL4 | Peptidase A1 domain-containing protein | T. asperellum | 372 |

TABLE 3
Non-limiting metalloproteases from Trichoderma

| UniProtKB | | | Length |
|---|---|---|---|
| No. | Protein names | Organism | [aa] |
| G0RT39 | Predicted protein | T. reesei | 442 |
| G0RKF5 | Predicted protein | T. reesei | 442 |
| G0RRB3 | Predicted protein | T. reesei | 594 |
| G0RBV9 | Acetylornithine deacetylase | T. reesei | 571 |
| G0RSV5 | Peptide hydrolase (EC 3.4.-.-) | T. reesei | 513 |
| G0RSY9 | Peptide hydrolase (EC 3.4.-.-) | T. reesei | 491 |
| G0RNF4 | Peptide hydrolase (EC 3.4.-.-) | T. reesei | 384 |
| G0RC20 | Peptide hydrolase (EC 3.4.-.-) | T. reesei | 395 |
| G0RF20 | Peptide hydrolase (EC 3.4.-.-) | T. reesei | 369 |
| G0RIL9 | Neutral protease 2 (EC 3.4.24.39) | T. reesei | 347 |
| | (Deuterolysin) | | |
| G0RX52 | Extracellular metalloproteinase (EC | T. reesei | 640 |
| | 3.4.24.-) (Fungalysin) | | |
| G0RF39 | Carboxypeptidase (EC 3.4.16.-) | T. reesei | 548 |
| A0A2H2Z803 | Metallocarboxypeptidase | T. parareesei | 398 |
| A0A2H2ZQT2 | Zinc carboxypeptidase | T. parareesei | 419 |
| A0A2H2ZSA8 | M20_dimer domain-containing protein | T. parareesei | 416 |
| A0A2H2Z059 | Peptide hydrolase (EC 3.4.-.-) | T. parareesei | 491 |
| A0A2H2Z2T6 | Peptide hydrolase (EC 3.4.-.-) | T. parareesei | 436 |
| A0A2H2ZN91 | Peptide hydrolase (EC 3.4.-.-) | T. parareesei | 513 |
| A0A2H3A2T4 | Peptide hydrolase (EC 3.4.-.-) | T. parareesei | 369 |
| A0A2H3A0S7 | Peptide hydrolase (EC 3.4.-.-) | T. parareesei | 384 |
| A0A2H2Z9D1 | Peptide hydrolase (EC 3.4.-.-) | T. parareesei | 372 |
| A0A2H3AC59 | Neutral protease 2 (EC 3.4.24.39) | T. parareesei | 347 |
| | (Deuterolysin) | | |
| A0A2H2ZSQ2 | Uncharacterized protein | T. parareesei | 646 |
| A0A2T4AN33 | Peptidase_M14 domain-containing protein | T. harzianum | 442 |
| A0A2T4AER1 | Peptidase_M14 domain-containing protein | T. harzianum | 422 |
| A0A2T4AA39 | M20_dimer domain-containing protein | T. harzianum | 418 |
| A0A2T3ZV96 | M20_dimer domain-containing protein | T. harzianum | 584 |
| A0A2T4AN00 | Probable Xaa-Pro aminopeptidase P | T. harzianum | 444 |
| A0A2T4AQC4 | Peptide hydrolase (EC 3.4.-.-) | T. harzianum | 491 |
| A0A2T3ZUJ0 | Peptide hydrolase (EC 3.4.-.-) | T. harzianum | 427 |
| A0A2T4AQI7 | Peptide hydrolase (EC 3.4.-.-) | T. harzianum | 522 |
| A0A2T3ZUP1 | Peptide hydrolase (EC 3.4.-.-) | T. harzianum | 369 |
| A0A2T4ACX3 | Peptide hydrolase (EC 3.4.-.-) | T. harzianum | 379 |
| A0A2T4AAN8 | Peptide hydrolase (EC 3.4.-.-) | T. harzianum | 398 |
| A0A2T4AFJ4 | Neutral protease 2 (EC 3.4.24.39) | T. harzianum | 347 |
| | (Deuterolysin) | | |
| A0A2T4A1D0 | Extracellular metalloproteinase (EC | T. harzianum | 638 |
| | 3.4.24.-) (Fungalysin) | | |
| A0A2T4A489 | Uncharacterized protein | T. harzianum | 673 |
| G9MNF3 | Peptidase_M14 domain-containing protein | T. virens | 422 |
| G9N5W5 | M20_dimer domain-containing protein | T. virens | 419 |
| G9MV10 | M20_dimer domain-containing protein | T. virens | 570 |
| G9NC25 | Peptide hydrolase (EC 3.4.-.-) | T. virens | 515 |
| G9MIS6 | Peptide hydrolase (EC 3.4.-.-) | T. virens | 379 |
| G9MWX4 | Peptide hydrolase (EC 3.4.-.-) (Fragment) | T. virens | 433 |
| G9N5Z0 | Peptide hydrolase (EC 3.4.-.-) | T. virens | 395 |
| G9MYA8 | Peptide hydrolase (EC 3.4.-.-) | T. virens | 369 |
| G9MP13 | Neutral protease 2 (EC 3.4.24.39) | T. virens | 347 |
| | (Deuterolysin) | | |
| G9MES4 | Extracellular metalloproteinase (EC | T. virens | 638 |
| | 3.4.24.-) (Fungalysin) | | |
| G9N8B6 | Uncharacterized protein | T. virens | 673 |
| G9P8Z8 | Peptidase_M14 domain-containing protein | T. atroviride | 437 |
| G9NZD3 | Peptidase_M14 domain-containing protein | T. atroviride | 420 |
| G9NFM3 | Peptidase_M14 domain-containing protein | T. atroviride | 441 |
| G9NGR6 | M20_dimer domain-containing protein | T. atroviride | 412 |
| G9NGL8 | M20_dimer domain-containing protein | T. atroviride | 558 |
| G9NTY8 | M20_dimer domain-containing protein | T. atroviride | 541 |
| G9NL69 | Probable Xaa-Pro aminopeptidase P | T. atroviride | 426 |
| G9P7L1 | Peptide hydrolase (EC 3.4.-.-) | T. atroviride | 492 |
| G9P7C7 | Peptide hydrolase (EC 3.4.-.-) | T. atroviride | 511 |
| G9PBQ8 | Peptide hydrolase (EC 3.4.-.-) | T. atroviride | 385 |
| G9PAW4 | Peptide hydrolase (EC 3.4.-.-) | T. atroviride | 428 |
| G9NHR8 | Peptide hydrolase (EC 3.4.-.-) | T. atroviride | 399 |
| G9NRV1 | Peptide hydrolase (EC 3.4.-.-) | T. atroviride | 369 |
| GYNZY0 | Neutral protease 2 (EC 3.4.24.39) | T. atroviride | 347 |
| | (Deuterolysin) | | |
| G9NXA4 | Extracellular metalloproteinase (EC | T. atroviride | 638 |
| | 3.4.24.-) (Fungalysin) | | |
| G9NNM8 | Uncharacterized protein | T. atroviride | 689 |
| A0A2T3YU17 | Peptidase_M14 domain-containing protein | T. asperellum | 555 |
| A0A2T3ZMC7 | Peptidase_M14 domain-containing protein | T. asperellum | 441 |
| A0A2T3ZIG7 | Peptidase_M14 domain-containing protein | T. asperellum | 419 |
| A0A2T3ZPE4 | Peptidase_M14 domain-containing protein | T. asperellum | 437 |
| A0A2T3Z3J5 | M20_dimer domain-containing protein | T. asperellum | 413 |
| A0A2T3Z3C5 | M20_dimer domain-containing protein | T. asperellum | 558 |
| A0A2T3ZML3 | Peptide hydrolase (EC 3.4.-.-) | T. asperellum | 492 |
| A0A2T3Z7G6 | Peptide hydrolase (EC 3.4.-.-) | T. asperellum | 384 |
| A0A2T3YYI1 | Peptide hydrolase (EC 3.4.-.-) | T. asperellum | 428 |
| A0A2T3YZR9 | Peptide hydrolase (EC 3.4.-.-) | T. asperellum | 369 |
| A0A2T3ZMN3 | Peptide hydrolase (EC 3.4.-.-) | T. asperellum | 511 |
| A0A2T3YTP5 | Carboxypeptidase (EC 3.4.16.-) | T. asperellum | 564 |
| A0A2T3ZHS6 | Neutral protease 2 (EC 3.4.24.39) | T. asperellum | 347 |
| | (Deuterolysin) | | |
| A0A2T3Z179 | Extracellular metalloproteinase (EC | T. asperellum | 638 |
| | 3.4.24.-) (Fungalysin) | | |
| A0A2T3YVP9 | Uncharacterized protein | T. asperellum | 690 |
| A0A2T3ZEF7 | Peptidase S1 domain-containing protein | T. asperellum | 255 |

TABLE 4
Non-limiting glutamate/glutamine proteases from Trichoderma

| UniProtKB | | | Length |
|---|---|---|---|
| No. | Protein names | Organism | [aa] |
| G0RXB5 | Predicted protein | T. reesei | 282 |
| A0A2H2ZSE0 | Uncharacterized protein | T. parareesei | 242 |
| A0A2H2ZKW9 | Aspartyl protease | T. parareesei | 395 |
| A0A2T4A7Z7 | Uncharacterized protein | T. harzianum | 264 |
| A0A2T3ZRV5 | Uncharacterized protein | T. harzianum | 221 |
| A0A2T4AJ17 | Peptidase A1 domain-containing protein | T. harzianum | 404 |
| A0A2T4AEU7 | Peptidase A1 domain-containing protein | T. harzianum | 395 |
| G9N465 | Uncharacterized protein | T. virens | 261 |
| G9NZH3 | Peptidase A1 domain-containing protein | T. atroviride | 395 |
| G9NTY0 | Uncharacterized protein | T. atroviride | 256 |
| A0A2T3Z0A4 | Uncharacterized protein | T. asperellum | 265 |
| A0A2T3ZI84 | Peptidase A1 domain-containing protein | T. asperellum | 395 |
| A0A2T3ZAN6 | Uncharacterized protein | T. asperellum | 256 |
| A0A2T3Z627 | Uncharacterized protein | T. asperellum | 243 |
| A0A2T3YS08 | Uncharacterized protein | T. asperellum | 266 |

TABLE 5
Non-limiting cysteine proteases from Trichoderma

| UniProtKB | | | Length |
|---|---|---|---|
| No. | Protein names | Organism | [aa] |
| G0RKG4 | Cysteine protease | T. reesei | 450 |
| A2H2ZRL7 | Cysteine protease | T. parareesei | 463 |
| A0A2H2ZB38 | Cysteine protease | T. parareesei | 431 |
| A0A2T4AVH5 | Uncharacterized protein | T. harzianum | 388 |
| A0A2T3ZUA1 | Carboxylic ester hydrolase (EC 3.1.1.-) | T. harzianum | 558 |
| G9MW72 | Uncharacterized protein | T. virens | 388 |
| G9MVA9 | Peptidase_M14 domain-containing protein | T. virens | 441 |
| G9P1Z5 | Uncharacterized protein | T. atroviride | 388 |
| A0A2T3ZLP7 | Uncharacterized protein | T. asperellum | 388 |

The proteases herein can be provided within a formulation (e.g., a protease formulation). This formulation, itself, can be employed as a reagent during hydrolysis processes or as a component within a biostimulant. The protease can be prepared in any useful manner.

In one instance, the protease can be isolated from an isolate obtained from a Trichoderma species, and the isolate can be obtained by fermentation. For instance, an initial inoculum having the Trichoderma strain may be maintained to provide a pure strain with minimal contaminants. In some embodiments, the initial inoculum is refreshed frequently and stored at 4° C. This initial inoculum can be grown on agar to provide the starting conidia (spores), which can then be washed from the surface of the culture with sterile saline and inoculated into culture media for fermentation (e.g., in a bioreactor).

In another instance, the fermentation broth is filtered to provide a filter cake, which can then be extruded to form granules. During extrusion and granulation, one or more carriers and/or additives can be added. Optionally, the filtrate (e.g., the protease) can be lyophilized and employed (e.g., in any formulation described herein).

In one embodiment, concentrated quantities of Trichoderma can be obtained using a liquid medium by inoculating pure Trichoderma species onto a solid agar plate (e.g., autoclaved potato dextrose agar (PDA)) for 1 week. Conidia (spores) can be washed from the surface of the culture with sterile saline and inoculated into semi-defined balanced media (SDBM, including molasses, urea, KH₂PO₄, MgSO₄·7H₂O, and sodium citrate, pH of 7.0). Cultures can be grown at 28° C., shaking for 24 hours. The Trichoderma biomass can be filtered (e.g., through 100 μm sieves). Trichoderma produced in this way can, in some instances, possess more than 50% colonization potential through 10 days of storage. Studies have demonstrated, however, that inclusion of additives, such as additional sugars or starches, metabolic inhibitors (such as CuSO₄), or kaolin can increase the longevity of spore-containing cultures of fungus. Blended strains of Trichoderma with combinations of these additives can be prepared to assess the overall colonization potential both before and after four months of storage. In use, Trichoderma strains can be refreshed from time to time. In one instance, from a PDA plate with fresh and uncontaminated colonies, a pre-inoculum can be prepared for both liquid and solid formulations.

Any useful fermentation method can be used, such as liquid fermentation, semi-solid fermentation, solid fermentation, and biofilm formation. In one instance, fermentation can include fermentation with a culture medium including rice husk, sugar beet pulp, sugar cane bagasse, glucose, glucose nitrate, maltose, maltose peptone, hydrolyzed corn, molasses, dextrose, potato dextrose, soy, starch, whey, or other agricultural products or byproducts of the food industry in solid forms and/or broth forms. Prior to inoculation of the Trichoderma species, the culture medium can be sterilized.

Depending on the final formulation, strains may be grown at a temperature between about 24-25° C. During the period of manufactory sporulation, the ecosystem can be controlled to avoid contamination from other fungi, such as penicillium. After sufficient production in the medium, the spores can be separated (e.g., centrifugation, air separation, filtration, etc.) and stored as active matter (e.g., such as in a solid formulation including the Trichoderma isolates). Alternatively, the liquid fermentation broth can be used as the active matter (e.g., such as in a liquid formulation including the Trichoderma isolates). In another embodiment, the fermentation broth can be filtered to provide a filter cake, which can then be processed to provide a solid formulation (e.g., granules, which can be obtained by granulation of the filter cake with one or more optional carriers and/or optional additives).

In some embodiments, a solid formulation of proteases from one or more Trichoderma species can be mixed with a solid carrier. If two or more species, are employed, then various ratios of the two proteases from each strain can be mixed with a solid carrier, as described herein.

Compositions and formulations can be tested in any useful manner, such as by using in vitro and in vivo assays. Non-limiting assays include enzymatic proteolytic assays, colony growth inhibition assays, conidial germination inhibition assays, viability assays, quantitative growth assessment of plant growth (e.g., as determined by plant height, number and/or quality of fruit, and the like), soil sample analysis (e.g., before, during, and after applying the formulation), total biomass measurements of plants, root growth determinations, and overall health monitoring of plants. In particular, in vitro assays can be conducted to test the enzymatic, proteolytic (or hydrolytic) activity of the protease for a detectable substrate.

Any of the protease compositions herein can be combined with a carrier to provide a formulation, which can be a liquid formulation, a solid formulation, or a semi-solid formulation. In some embodiments, a carrier is used with a Trichoderma or Trichoderma protease composition for hydrolyzing plant protein feedstock to produce a biostimulant formulation. Such carriers may be solid material (e.g., such as agar, particles, etc.) or a liquid (e.g., such as water or vegetable oil). The liquid carrier may also serve as a diluent.

In general, a carrier is an inert component within a formulation. While the presence of the inert ingredient may be helpful in using or delivering a formulation, the inert ingredient does not necessarily impart biofungicidal, biostimulant, or proteolytic effect. For instance, a protease formulation may include water or buffer as a carrier, which may assist in dispersing the protease into a feedstock to provide a liquid-based hydrolysis product; but the water and buffer itself does not provide bioactive effect.

A liquid formulation can include a liquid carrier, such as water, a vegetable oil, or a mineral oil. The liquid formulation can include any useful form, such as a concentrate (e.g., an emulsifiable concentrate) or an emulsion. In some embodiments, the liquid carrier is configured to stabilize formulation, thereby providing a shelf stable product. The liquid formulation can be processed to inactivate proteases. Inactivation processes can include use of thermal shock, exposure to light or radiation, and the like.

A solid formulation can include a solid particle. For example, the protease can be provided within a solid formulation. Note that whereas the protease formulation can be in liquid or solid form upon adding to a feedstock, the resulting hydrolysis product (e.g., as described herein) is typically in a liquid form. The solid particle can include a water-soluble, a wettable, or a water-dispersible particle, which can be dissolved or dispersed upon addition of an aqueous solvent. The particle can be of any form, such as a powder (e.g., a wettable powder, a freeze-dried powder, a spray-dried powder, a flowable powder, and the like), a pellet, or a granule (e.g., a microgranule, and the like). Such particles can be formed of a solid carrier such as a solid phase, a clay (e.g., kaolin), a sugar alcohol (e.g., sorbitol), a sugar, a saccharide, or a polysaccharide (e.g., cellulose). In another instance, the particle can be encapsulated with a coating, which can have one or more layers and in which each layer can include one or more solid carriers. Useful technologies and methodologies to prepare, synthesize, and modify nanoparticles can be employed to provide such coatings.

The concentration of the protease may depend on whether the formulation is a solid formulation or a liquid formulation. Within a solid formulation, the concentration of the protease(s) may be about 0.05 to 20% (w/w) of a solid formulation.

The protease formulation can include one or more proteases from a Trichoderma species, and the remaining weight of the formulation can be one or more carriers (e.g., any described herein), which can optionally include any additives described herein. Non-limiting solid carriers include clays (e.g., kaolin), saccharides, polysaccharides (e.g., cellulose), and the like.

Within a liquid form of the protease formulation, the concentration of the protease(s) may be from about 0.1 to 100% (w/v) or (v/v) of a liquid formulation. Such concentrations can be for each protease from each Trichoderma species within the formulation or for the combined concentration of all proteases from all Trichoderma species within the formulation. The remaining volume of the formulation can be one or more carriers (e.g., any described herein), which can optionally include any additives described herein. Non-limiting liquid carriers include mineral oil, water, and the like.

Carriers can include any useful combination of components, such as binders, encapsulating materials, carbonaceous matter, fillers, desiccants, liquids, dispersants, and the like. Non-limiting binders can include cellulose esters (e.g., methylcellulose and hydroxypropyl cellulose), organic silicates (e.g., organosilicon esters, gums, alginate, and the like), polyalkylene oxide (e.g., polyethylene oxide), polyvinyl alcohol, polyvinyl acetate, and starch.

Non-limiting encapsulating materials include alginate, chitosan, carrageenan, cellulose, dextrin, glucan, gums (locust bean, gellan gum, xanthan gum, etc.), gelatin, whey protein, starch, vegetable or microbial gum, and combinations thereof.

Non-limiting carbonaceous matter (e.g., carbonaceous particulate solid matter) can include alginate granules, agricultural byproducts (e.g., rice), aquatic products (e.g., seaweed or kelp), barley grain, bituminous coal, leonardite (e.g., Agrolig®), muck soil activated carbon and charcoal, organic soil, peat, peat-like substances (e.g., peat moss), pyrophyllite (e.g., Pyrax®, anhydrous aluminum silicate), shale, soft coal, sphagnum moss, tree bark granules, wheat bran, wood bark compost, and combinations thereof.

Non-limiting fillers can include alginic acid, cellulose, chitin, clays, cyclodextrins, diatomaceous earths, dextrose granules or powders, gelatin, ground agricultural products, maltose-dextrose granules or powders, mineral powder (e.g., bentonite, cation clay, diatomaceous earth, kaolin, talc, and the like), porous beads or powders, porous wood products, silica, silicates, sucrose granules or powders, talc, vermiculite, zeolite, and combinations thereof. Such fillers may be useful, e.g., for improving seed coating and/or enhancing water absorption within the solid formulation.

Non-limiting desiccants include a sugar (e.g., a non-reducing sugar, a disaccharide, trehalose, sucrose, and the like), a polyol (e.g., glycerol, triethylene glycol, and the like), sugar alcohol (e.g., mannitol, sorbitol, and the like), calcium sulfate, silica, and combinations thereof.

Non-limiting liquids include water, buffer, oil (e.g., mineral oil, corn oil, olive oil, palm oil, palm kernel oil, peanut oil, rapeseed oil, rice bran oil, soybean oil, sunflower oil, vegetable oil, and the like), a non-aqueous solvent, an organic solvent (e.g., alcohol), polyethylene glycols (e.g., PEG 200, PEG 300, PEG 400, etc.), propylene glycols (e.g., PPG-9, PPG-10, PPG-17, PPG-20, PPG-26, etc.), a polyethylene glycol-polypropylene glycol copolymer, an ethoxylated alcohol, an aqueous solvent (e.g., water or a buffer), polysorbates (e.g. polysorbate 20, polysorbate 40, polysorbate 60, polysorbate 80, etc.), silicones (siloxanes, trisiloxanes, etc.), and combinations thereof. Non-limiting dispersants include alcohol ethoxylates, naphthalene sulfonates, vinylpyrrolidone polymers, nonionic surfactants, ionic surfactants such as cationic or anionic surfactants, and the like.

Such formulations can further include one or more optional additives, such as any described herein. Additives can include added active ingredients, which may improve or enhance the formulation. For instance, such an additive can include zinc, manganese, and the like. For example, a protease may require the presence of a cofactor or a metal ion for maximal proteolytic activity, and the additive can include such a cofactor or metal ion.

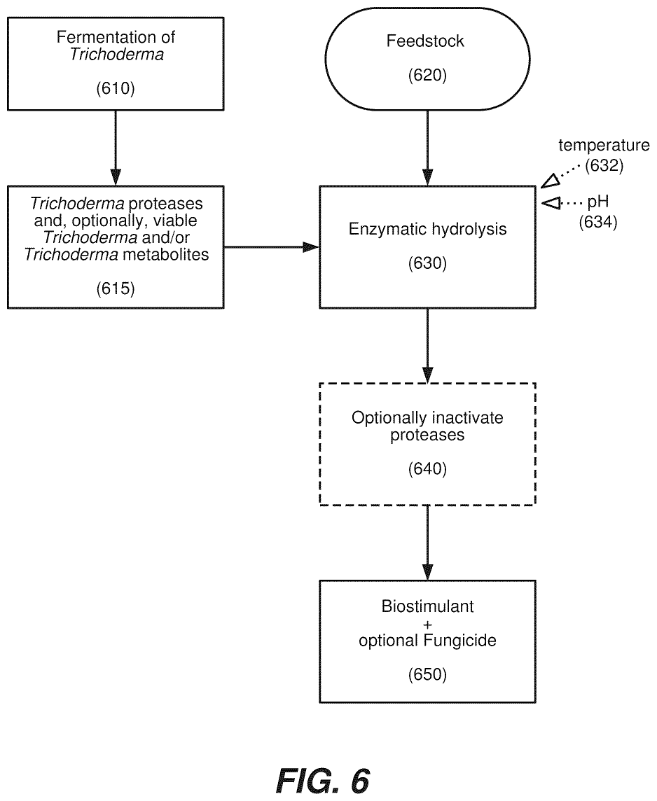

Proteases from Trichoderma can be used to treat a feedstock and provide a biostimulant formulation. One non-limiting process is provided in FIG. 6, which includes an operation 610 for fermentation of Trichoderma within a culture. In operation 615, one or more components can be obtained from the culture. For instance, such components can include Trichoderma-derived proteases and, optionally, viable Trichoderma and/or Trichoderma-derived metabolites. Such Trichoderma-derived components, or even viable Trichoderma itself, can be combined with feedstock 620. In one embodiment, an operation 630 includes enzymatic hydrolysis of the feedstock by using Trichoderma or Trichoderma-derived components. For example, Trichoderma-derived proteases can be used to lyse proteins present within the feedstock. During enzymatic hydrolysis, various process conditions can be optimized, such as temperature 632, pH 634, and the like.

Optionally, an operation 640 can be conducted to inactivate the proteases. Any useful inactivation method can be employed, such as use of thermal shock or radiation.

In operation 650, the resulting formulation or composition can be characterized as a biostimulant that can optionally include a fungicide. The biostimulant can include any described herein, such as a hydrolysis product or a component of a hydrolysis product (e.g., an amino acid and an oligopeptide, wherein the amino acid and the oligopeptide are derived from a plant-based feedstock). The fungicide can include metabolites, in which Trichoderma can release metabolites that create a fungicidal effect. Such an effect is observed even when proteases are inactivated, so it is not necessarily the proteases that provide the fungicide effect but the secondary metabolites.

### Trichoderma Species

The feedstocks herein can be treated with protease(s) from any Trichoderma species, as well as combinations of Trichoderma species. For instance, by such treatment, the protease(s) from the Trichoderma species can enzymatically hydrolyze the plant protein or feedstock. Non-limiting Trichoderma species include T. parareesei species, T. reesei species, T. virens species, T. aureoviride species, T. atroviride species, T. harzianum species, T. asperellum species, T. longibrachiatum species, as well as combinations thereof.

In some embodiments, the T. parareesei species can include strain T6 or strain iQB 6. In other embodiments, the species T. parareesei is characterized as CECT Accession No. 20102 (Colección Española de Cultivos Tipo [Spanish Type Culture Collection], Valencia, Spain), IMI No. 113135 (International Mycological Institute (IMI), now the Centre for Agriculture and Bioscience International, Wallingford, England), or IHEM Accession No. 5436 (Institute of Hygiene and Epidemiology, Mycology Laboratory (IHEM), now the Belgian Co-ordinated Collections of Micro-organisms, Brussels, Belgium). Yet other names for this species can include T. atrobrunneum F. B. Rocha, P. Chaverri & W. Jaklitsch 2014, T. aureoviride, T. aureoviride (Rifai), T. harzianum (Rifai), T. reesei, or Hypocrea jecorina.

In some instances, the species T. parareesei is defined by an intergenic sequence, such as an internal transcribed spacer (ITS) sequence. Non-limiting ITS sequences include GenBank Accession No.: AJ251698, Trichoderma reesei internal transcribed spacer 1 (ITS1), isolate 6 (SEQ ID NO:110) or a fragment thereof, as well as a nucleic acid sequence having at least 80%, 85%, 90%, or 95% sequence identity to SEQ ID NO:110 or a fragment thereof.

In other instances, the species T. parareesei is defined by the gene sequence for translation elongation factor 1-α (tef1). In one embodiment, the tef1 sequence includes GenBank Accession No.: AJ563621, Hypocrea jecorina partial tef1 gene for translation elongation factor 1, exons 5-6, strain IMI 113135 (SEQ ID NO:111) or a fragment thereof, as well as a nucleic acid sequence having at least 80%, 85%, 90%, or 95% sequence identity to SEQ ID NO:111 or a fragment thereof.

In another embodiment, the tef1 sequence includes GenBank Accession No.: KF699130, Trichoderma parareesei strain T6 translation elongation factor 1-alpha (tef1) gene, partial cds (SEQ ID NO:112) or a fragment thereof, as well as a nucleic acid sequence having at least 80%, 85%, 90%, or 95% sequence identity to SEQ ID NO:112 or a fragment thereof.

In yet another embodiment, the species T. parareesei is defined by the gene sequence for calmodulin (call), such as that provided in GenBank Accession No.: KF699131, Trichoderma parareesei strain T6 calmodulin (CAL1) gene, partial cds (SEQ ID NO:113) or a fragment thereof, as well as a nucleic acid sequence having at least 80%, 85%, 90%, or 95% sequence identity to SEQ ID NO:113 or a fragment thereof.

The presence of certain gene sequences can be determined by using primers to bind and to amplify targeted gene sequences. Non-limiting primers can include those for the fourth large intron of tef1 using primers EF1-728F (5′-CATCGAGAAGTCGAGAAGG-3′, SEQ ID NO:114) and TEF1-LLErev (5′-AACTGCAGGCAATGTGG-3′, SEQ ID NO:115); and those for a fragment of call using primers CAL-228F (5′-GAGTCAAGGAGGCCTTCTCCC-3′, SEQ ID NO:116) and CAL-737R (5′-CATCTITCTGGCCATCATGG-3′, SEQ ID NO:117). Other non-limiting methods for identifying T. parareesei are described in Rubio M B et al., “Identifying beneficial qualities of Trichoderma parareesei for plants,” Appl. Environ. Microbiol. 2014; 80(6): 1864-1873, which is incorporated herein by reference in its entirety.

In some embodiments, the T. virens species can include strain T59 or strain iQB 59. In other embodiments, the species T. virens is characterized as BioSample Accession No. SAMN00150230 (National Center for Biotechnology Information, Bethesda, Maryland).

In other instances, the species T. virens is characterized by an ITS sequence including GenBank Accession No.: AJ517317, Trichoderma virens internal transcribed spacer 1 (ITS1) (SEQ ID NO:120) or a fragment thereof, as well as a nucleic acid sequence having at least 80%, 85%, 90%, or 95% sequence identity to SEQ ID NO:120 or a fragment thereof.

In yet other instances, the species T. virens is characterized by a thioredoxin-like protein gene dim1 sequence including GenBank Accession No.: FJ788527.1, Hypocrea virens strain T59 thioredoxin-like protein (Dim1) gene (SEQ ID NO:121) or a fragment thereof, as well as a nucleic acid sequence having at least 80%, 85%, 90%, or 95% sequence identity to SEQ ID NO:121 or a fragment thereof.

In yet other embodiments, the species T. virens is characterized by the presence of a thioredoxin-like protein (Dim1) or a gene expressing Dim1 (dim1), in which the Dim1 protein includes GenBank Accession No.: ACY01406.1, thioredoxin-like protein [Trichoderma virens], amino acids 1-143 (SEQ ID NO:122) or amino acids 6-137 (SEQ ID NO:123) or a fragment thereof, as well as a polypeptide sequence having at least 80%, 85%, 90%, or 95% sequence identity to one of SEQ ID NO:122, SEQ ID NO:123, or a fragment of any of these.

The presence of certain gene sequences can be determined by using primers to bind and to amplify targeted gene sequences. Non-limiting primers can include those for the dim1 gene using primers TRX-5 (5′-GAAGAGGATCGTCTCGTCGTC-3′, SEQ ID NO:124) and TRX-3 (5′-TCAGGAACCTCGTCAATGTCG-3′, SEQ ID NO:125). Other non-limiting methods for identifying T. virens are described in Morin-Diez M E et al., “TvDim1 of Trichoderma virens is involved in redox-processes and confers resistance to oxidative stresses,” Curr. Genet. 2010; 56: 63-73, which is incorporated herein by reference in its entirety.

In one embodiment, the isolate includes the species T. atroviride. Such species can include strain T11 or strain iQB 11. In particular embodiments, the species T. atroviride is characterized as CECT Accession No. 20498 or IMI No. 352941. Yet other names for this species can include T. harzianum or T. harzianum (Rifai).

In some instances, the species T. atroviride is defined by an ITS sequence, such as GenBank Accession No.: AJ224008, Trichoderma harzianum 5.8S rRNA and ITS1 and ITS2 DNA, isolate 11 (SEQ ID NO:130) or a fragment thereof, as well as a nucleic acid sequence having at least 80%, 85%, 90%, or 95% sequence identity to SEQ ID NO:130 or a fragment thereof.

In other instances, the species T. atroviride is defined by the gene sequence for tef1, such as GenBank Accession No.: AJ563609, Trichoderma cf. viride partial tef1 gene for translation elongation factor 1, exons 5-6, strain IMI 352941 (SEQ ID NO:131) or a fragment thereof, as well as a nucleic acid sequence having at least 80%, 85%, 90%, or 95% sequence identity to SEQ ID NO:131 or a fragment thereof.

In another embodiment, the isolate includes the species T. asperellum. Such species can include strain T25 or strain iQB 25. In particular embodiments, the species T. asperellum is characterized as CECT Accession No. 20178, CECT Accession No. 2941, or IMI No. 296237. Yet other names for this species can include T. viride.

In some instances, the species T. asperellum is defined by an ITS sequence, such as GenBank Accession No.: AJ223773, Trichoderma viride 5.8S rRNA gene, ITS1 and ITS2, isolate 25 (SEQ ID NO:140) or a fragment thereof, as well as a nucleic acid sequence having at least 80%, 85%, 90%, or 95% sequence identity to SEQ ID NO:140 or a fragment thereof.

In other instances, the species T. asperellum is defined by the gene sequence for tef1, such as GenBank Accession No.: AJ563611, Trichoderma asperellum partial tef1 gene for translation elongation factor 1, exons 5-6, strain IMI 296237 (SEQ ID NO:141) or a fragment thereof, as well as a nucleic acid sequence having at least 80%, 85%, 90%, or 95% sequence identity to SEQ ID NO:141 or a fragment thereof.

The presence of certain gene sequences can be determined by using primers to bind to amplify targeted gene sequences. Non-limiting primers can include those for the ITS1 region using a 5′ primer (5′-TCCGTAGGTGAACCTGCGG-3′, ITS1 primer, SEQ ID NO:142) and a 3′ primer (5′-GCTGCGTCTCATCGATGC-3′, ITS2 primer, SEQ ID NO:143); those for the ITS1 region, ITS2 region, and the 5.8S rDNA gene using a 5′ primer (5′-TCCGTAGGTGAACC TGCGG-3′, ITS1 primer, SEQ ID NO:142) and a 3′ primer (5′-TCCTCCGCTTATGATATGC-3′, ITS4 primer, SEQ ID NO:144); those for the 5.8S rDNA gene and the ITS2 region using a 5′ primer (5′-GCATCGATGAAGAACGCAGC-3′, ITS3 primer, SEQ ID NO:145) and a 3′ primer (5′-TCCTCCGCTATGATATGC-3′, ITS4 primer, SEQ ID NO:144); and those for the tef1 gene using primers tef1fw (5′-GTGAGCGTGGTATCACCA-3′, SEQ ID NO:146) and tef1rev (5′-GCCATCCTTGGAGACCAGC-3′, SEQ ID NO:147). In particular embodiments, the primer pair ITS1 (SEQ ID NO:142) and ITS4 (SEQ ID NO:144) is employed to provide amplified PCR products that range from 563 to 602 base pairs (bp), in which the products contain the ITS1 region, the ITS2 region, and the 5.8S rDNA gene and also 11 bp of the 3′ end of the 18S rDNA gene and 36 bp of the 5′ end of the 28S rDNA gene.

In yet other instances, the Trichoderma species is characterized by the presence of a thioredoxin-like protein (Dim1) gene sequence including GenBank Accession No.: FJ788527.1, Hypocrea virens strain T59 thioredoxin-like protein (Dim1) gene (SEQ ID NO:121) or a fragment thereof, as well as a nucleic acid sequence having at least 80%, 85%, 90%, or 95% sequence identity to SEQ ID NO:121 or a fragment thereof. In other embodiments, the Trichoderma species is characterized by the presence of a thioredoxin-like protein (Dim1) or a gene expressing Dim1, in which the Dim1 protein includes GenBank Accession No.: ACY01406.1, thioredoxin-like protein [Trichoderma virens], amino acids 1-143 (SEQ ID NO:122) or amino acids 6-137 (SEQ ID NO:123) or a fragment thereof, as well as a polypeptide sequence having at least 80%, 85%, 90%, or 95% sequence identity to one of SEQ ID NO:122, SEQ ID NO:123, or a fragment of any of these.

Non-limiting Trichoderma species expressing Dim1, or a homolog thereof, includes T. atroviride T11 (e.g., as described herein), T. asperellum T25 (e.g., as described herein), T. harzianum T34, T. harzianum T22, and T. longibrachiatum T52.

T. harzianum T34 can be characterized as CECT Accession No. 2413 or by an ITS sequence, including GenBank Accession No.: AF278790 (SEQ ID NO:150) or a fragment thereof, as well as a nucleic acid sequence having at least 80%, 85%, 90%, or 95% sequence identity to SEQ ID NO:150 or a fragment thereof.

T. harzianum T22 can be characterized as American Type Culture Collection Accession No. 20847 (ATCC® 20847™), which has recently been identified as Trichoderma afroharzianum. T. harzianum T22 can be characterized by an ITS sequence or by the gene sequence for tef1. In one embodiment, the ITS sequence includes GenBank Accession No.: FJ545255 (SEQ ID NO:151) or a fragment thereof, as well as a nucleic acid sequence having at least 80%, 85%, 90%, or 95% sequence identity to SEQ ID NO:151 or a fragment thereof. In another embodiment, the tef1 sequence includes GenBank Accession No.: KU933430 (SEQ ID NO:152) or a fragment thereof, as well as a nucleic acid sequence having at least 80%, 85%, 90%, or 95% sequence identity to SEQ ID NO:152 or a fragment thereof.

T. longibrachiatum T52 can be characterized by an ITS sequence including GenBank Accession No.: AJ251702 (SEQ ID NO:153) or a fragment thereof, as well as a nucleic acid sequence having at least 80%, 85%, 90%, or 95% sequence identity to SEQ ID NO:153 or a fragment thereof. One or more of any of the species herein can be used alone or combined to be used together.

### Biostimulant Compositions and Formulations

The biostimulant compositions and formulations herein can be any derived from a feedstock including plant proteins, after treatment with protease(s) from a Trichoderma species. In certain embodiments, a biostimulant composition includes two or more amino acids and one or more micronutrients.

The biostimulant compositions in accordance with certain disclosed embodiments can be derived from feedstock that includes a plant-based protein source. Plant-based protein sources may be selected based on their high organic matter content. These by-products can be selected by virtue of their high organic matter content, mainly proteins, and have been characterized to carry out enzymatic hydrolysis processes, obtaining said biostimulant products. Through hydrolytic processes, the functional properties of organic matter contained in agro-industrial organic by-products can be modified, which provides them with a greater capacity for agricultural application by increasing their bioavailability.

Either or both of the amino acids and oligopeptides may originate from a plant-based protein source. Some biostimulants can contain other components from a plant source, such as secondary metabolites, phytohormones, micronutrients, and/or macronutrients. Certain biostimulant compositions described herein are in liquid form or have components that are suspended in liquids. Yet other biostimulant compositions described herein are in solid form or have solid components.

The biostimulant compositions can be characterized by an amino acid profile. The amino acid profile can differ, depending on the starting raw material and hydrolysis conditions. Additionally, some raw materials can generate different peptide profiles; and some peptide profiles (oligopeptides and/or polypeptides) have greater or lesser beneficial properties, such as nutrient, antimicrobial, and antibacterial capacity. When controlled enzymatic hydrolysis of proteins is carried out, a balance can be obtained between amino acids in free form and as peptides, which gives the hydrolysate a significant nutritional role as a biostimulant that stimulates the growth and development of plants and crops and that increases the microbiological activity of the soil. The amino acids and the low molecular weight peptides that make them up can be nutritious substances that are easily absorbed and assimilated by plants, both by foliar and root routes, and can be transported to the plant's organs, such as buds, flowers, fruits.

Various types of amino acids may be present in the biostimulant composition. An amino acid profile may be characterized by relative amounts or concentrations of individual amino acids (e.g., proline, alanine, arginine) and/or by the relative amounts or concentrations of classes or types of amino acids.

In some embodiments, amino acids may be non-proteinogenic amino acids. In some embodiments, amino acids may be proteinogenic amino acids. For example, in some embodiments, any one or more of the following types of amino acids are present: aliphatic amino acids, aromatic amino acids, non-polar and neutral amino acids, polar and neutral amino acids, acidic and polar amino acids, and basic and polar amino acids. The amino acids in a biostimulant composition may be proteinogenic or non-proteinogenic (e.g., taurine and omithine). A biostimulant composition may have at least one of the following amino acids at greater than a trace concentration: aspartic acid with asparagine, glutamic acid with glutamine, glycine, serine, threonine, histidine, tyrosine, arginine, alanine, methionine, valine, tryptophan, phenylalanine, asparagine, glutamine, isoleucine, leucine, proline, hydroxyproline, omithine, and taurine. In some embodiments, the biostimulant composition includes at least glycine and lysine. In other embodiments, the biostimulant includes at least glutamic acid, glutamine, glycine, and lysine.

The below concentration percentages are percentages by weight of each amino acid divided by the total weight of the free amino acid component in the biostimulant composition (e.g., 33% glutamic acid and glutamine means that of the weight of amino acids in the biostimulant composition, 33% of the weight is glutamic acid and glutamine). Ranges in Table 6 are approximate concentration ranges for each amino acid produced from one example of a raw feedstock material.

TABLE 6
Non-limiting amino acid profile

| Amino Acid | Minimum | Maximum |
|---|---|---|
| Aspartic acid and asparagine | About 0.05% | About 0.3% |
| Glutamic acid and glutamine | About 30% | About 40% |
| Glycine | About 10% | About 20% |
| Serine | About 0.1% | About 0.5% |
| Threonine | About 0.3% | About 0.7% |
| Histidine | About 0.01% | About 0.1% |
| Tyrosine | About 0.01% | About 0.2% |
| Arginine | About 0.1% | About 0.5% |
| Alanine | About 0.3% | About 0.7% |
| Methionine | About 0.01% | About 0.1% |
| Valine | About 0.1% | About 0.5% |
| Tryptophan | About 0.1% | About 0.5% |
| Phenylalanine | About 0.1% | About 0.5% |
| Asparagine | About 0.1% | About 0.5% |
| Glutamine | About 0.01% | About 0.1% |
| Isoleucine | About 0.1% | About 0.5% |
| Leucine | About 0.3% | About 0.7% |
| Lysine | About 40% | About 60% |
| Proline | About 0.01% | About 0.1% |
| Hydroxyproline | About 0.01% | About 0.1% |
| Ornithine | About 0.01% | About 0.1% |
| Taurine | About 0.01% | About 0.1% |

In various embodiments, glutamine, histidine, hydroxyproline, methionine, omithine, proline, taurine, tyrosine, aspartic acid and asparagine, arginine, asparagine, phenylalanine, serine, tryptophan, valine, isoleucine, alanine, leucine, and threonine may be secondary amino acid components. In various embodiments, lysine, glycine, glutamic acid, and glutamine may be primary amino acid components.

In some embodiments, the free amino acid component of a biostimulant composition includes: (a) one or more primary amino acid components selected from the group consisting of lysine, glycine, glutamic acid, and glutamine; and (b) one or more secondary amino acid components selected from the group consisting of glutamine, histidine, hydroxyproline, methionine, omithine, proline, taurine, tyrosine, aspartic acid, asparagine, arginine, phenylalanine, serine, tryptophan, valine, isoleucine, alanine, leucine, and threonine.

In other embodiments, the free amino acid component of a biostimulant composition includes: (a) one or more primary amino acid components selected from the group consisting of lysine, glycine, glutamic acid, and glutamine; and (b) one or more secondary amino acid components selected from the group consisting of alanine, leucine, and threonine. In yet other embodiments, the free amino acid component of a biostimulant composition includes: (a) one or more primary amino acid components selected from the group consisting of lysine, glycine, glutamic acid, and glutamine; and (b) one or more secondary amino acid components selected from the group consisting of tyrosine, aspartic acid, asparagine, arginine, phenylalanine, serine, tryptophan, valine, and isoleucine.

In various embodiments, the free amino acid component of a biostimulant composition has less than about 0.1% histidine, less than about 0.1% methionine, less than about 0.1% glutamine, less than about 0.1% proline, less than about 0.1% hydroxyproline, less than about 0.1% omithine, less than about 0.1% taurine by weight, or any combination of these. In some embodiments, the free amino acid component of a biostimulant composition has less than about 0.1% histidine by weight, or about 0.01 (w/w) % to 0.1 (w/w) % histidine, in which (w/w) % indicates the weight of the target amino acid versus the total weight of free amino acid components in the biostimulant composition. In some embodiments, the free amino acid component of a biostimulant composition has less than about 0.1 (w/w) % methionine, or about 0.01 (w/w) % to 0.1 (w/w) % methionine. In some embodiments, the free amino acid component of a biostimulant composition has less than about 0.1 (w/w) % glutamine, or about 0.01 (w/w) % to 0.1 (w/w) % glutamine. In some embodiments, the free amino acid component of a biostimulant composition has less than about 0.1 (w/w) % proline, or about 0.01 (w/w) % to 0.1 (w/w) % proline. In some embodiments, the free amino acid component of a biostimulant composition has less than about 0.1 (w/w) % hydroxyproline, or about 0.01 (w/w) % to 0.1 (w/w) % hydroxyproline. In some embodiments, the free amino acid component of a biostimulant composition has less than about 0.1 (w/w) % ornithine, or about 0.01 (w/w) % to 0.1 (w/w) % omithine. In some embodiments, the free amino acid component of a biostimulant composition has less than about 0.1 (w/w) % taurine, or about 0.01 (w/w) % to 0.1 (w/w) % taurine.

In some embodiments, about 0.01% to 0.3% of the free amino acid components in the biostimulant composition is aspartic acid and asparagine by weight. In some embodiments, the free amino acid component of a biostimulant composition has about 0.01% to 0.2% tyrosine by weight.

In various embodiments, the free amino acid component of a biostimulant composition has about 0.1% to 0.5% of each of serine, arginine, isoleucine, valine, tryptophan, phenylalanine, and asparagine by weight. In some embodiments, the free amino acid component of a biostimulant composition has about 0.1 (w/w) % to 0.5 (w/w) % serine or arginine or valine or tryptophan or phenylalanine or asparagine or isoleucine.

In various embodiments, the free amino acid component of a biostimulant composition has about 0.3% to 0.7% of each of threonine, alanine, and leucine by weight. In some embodiments, the free amino acid component of a biostimulant composition includes about 0.3 (w/w) % to 0.7 (w/w) % threonine or alanine or leucine.

In various embodiments, the free amino acid component of a biostimulant composition has mostly lysine, or about 50 (w/w) % or more lysine. In various embodiments, the free amino acid component of a biostimulant composition includes mostly glycine, glutamic acid and glutamine, and lysine. In some embodiments, about 30% to 40% of the free amino acid component of a biostimulant composition is glutamic acid and glutamine. In some embodiments, about 10% to 20% of the free amino acid component of a biostimulant composition is glycine. In some embodiments, about 40% to 60% of the free amino acid component of a biostimulant composition is lysine.

The biostimulant composition may include alpha amino acids. The biostimulant composition may include L-alpha amino acids. The biostimulant composition may include basic amino acids. The biostimulant composition may include aliphatic amino acids. The biostimulant composition may include charge-neutral polar amino acids.

The biostimulant composition may include one or more oligopeptides. An oligopeptide may facilitate delivering nutrients to plants and/or moving nutrients within plants.

The biostimulant composition may optionally include one or more non-amino acid and non-peptide components from the plant-based protein source. Examples of such additional plant-based components include phytohormones and secondary metabolites. Examples of phytohormones include cytokinins, abscisic acid, jasmonates, auxins, and phenolics. Examples of cytokinins include but are not limited to trans-zeatin riboside (tZR), dihydrozeatin riboside (DZR), cis-zeatin (cZ), cis-zeatin riboside (cZR), isopentenyl adenine (iP), isopentenyl adenosine (iPR), 2-methylthio zeatin (MeS-Z), and 2-methylthio isopentenyl adenine (MeS-iP). Example abscisic acids include abscisic acid (ABA), phaseic acid (PA), dihydrophaseic acid (DPA), and 9-hydroxy-ABA (90H-ABA). Example jasmonates include jasmonic acid (JA) and jasmonic acid isoleucine (JA-Ile). Examples of auxins include indole-3-acetic acid (IAA), oxo-indole-3-acetic acid (OxlAA), and indole-3-acetamide (IAM). Examples of phenolics include salicylic acid (SA) and phenylacetic acid (PAA).

In some embodiments, the concentration of one or more cytokinins in the composition is about 0.5 pmol/ml to 15 pmol/ml. In some embodiments, the concentration of tZR is about 0.1 pmol/ml to 0.4 pmol/ml. In some embodiments, the concentration of DZR is about 0.5 pmol/ml to 1.2 pmol/ml. In some embodiments, the concentration of cZ is about 6 pmol/ml to 8 pmol/ml. In some embodiments, the concentration of cZR is about 1 pmol/ml to 2 pmol/ml. In some embodiments, the concentration of iP is about 10 pmol/ml to 15 pmol/ml. In some embodiments, the concentration of iPR is about 1 pmol/ml to 2 pmol/ml. In some embodiments, the concentration of MeS-Z is about 4 pmol/ml to 6 pmol/ml. In some embodiments, the concentration of MeS-iP is about 0.5 pmol/ml to 0.1 pmol/ml.

In one example, the concentration of tZR is about 0.2 pmol/ml. In one example, the concentration of DZR is about 1 pmol/ml. In one example, the concentration of cZ is about 8 pmol/ml. In one example, the concentration of cZR is about 2 pmol/ml. In one example, the concentration of iP is about 14 pmol/ml. In one example, the concentration of iPR is about 1 pmol/ml. In one example, the concentration of MeS-Z is about 5 pmol/ml. In one example, the concentration of MeS-iP is about 1 pmol/ml.

In some embodiments, the concentration of certain ABAs may range from about 0.1 pmol/ml to 2800 pmol/ml. In some embodiments, the concentration of ABA is about 3 pmol/ml to 5 pmol/ml. In some embodiments, the concentration of PA is about 0.1 pmol/ml to 0.2 pmol/ml. In some embodiments, the concentration of DPA is about 2500 pmol/ml to 2800 pmol/ml. In some embodiments, the concentration of 90H-ABA is about 0.5 pmol/ml to 1.0 pmol/ml.

In some embodiments, the concentration of ABA is about 4 pmol/ml. In some embodiments, the concentration of PA is about 0.1 pmol/ml. In some embodiments, the concentration of DPA is about 2700 pmol/ml. In some embodiments, the concentration of 90H-ABA is about 0.7 pmol/ml.

In some embodiments, the concentration of certain jasmonates may range from about 0.1 pmol/ml to 3 pmol/ml. In some embodiments, the concentration of JA is about 2 pmol/ml to 3 pmol/ml. In some embodiments, the concentration of JA-Ile is about 0.1 pmol/ml to 0.4 pmol/ml.

In one example, the amount of JA is about 3 pmol/ml. In one example, the amount of JA-Ile is about 0.3 pmol/ml.

In some embodiments, the content of certain auxins may range from about 3 pmol/ml and about 20 pmol/ml. In some embodiments, the amount of IAA is about 15 pmol/ml to 20 pmol/ml. In some embodiments, the amount of OxIAA is about 4 pmol/ml to 5 pmol/ml. In some embodiments, the amount of IAm is about 3 pmol/ml to 5 pmol/ml.

In some embodiments, the amount of IAA is about 18 pmol/ml. In some embodiments, the amount of OxIAA is about 5 pmol/ml. In some embodiments, the amount of IAM is about 5 pmol/ml.

In some embodiments, the content of certain phenolics is about 150 pmol/ml to 50000 pmol/ml. In some embodiments, phenolics are the majority phytohormone of all phytohormones in the biostimulant composition. In some embodiments, the amount of SA is about 150 pmol/ml to 200 pmol/ml. In some embodiments, the amount of PAA is about 40000 pmol/ml to 50000 pmol/ml.

In some embodiments, the amount of SA is about 182 pmol/ml. In some embodiments, the amount of PAA is about 46000 pmol/ml.

In some embodiments, the portion of the biostimulant composition having phytohormones may be predominantly abscisic acids and phenolics. In some embodiments, phenolics are the majority component of phytohormones in a biostimulant composition.

Biostimulant compositions may include nutrients such as micronutrients and/or macronutrients, some of which are from the plant-based protein source, and some of which are added to the biostimulant composition to enhance the functions of the biostimulant composition.

Examples of nutrients include but are not limited to calcium, sulfur, magnesium, carbon, oxygen, hydrogen, iron, manganese, boron, molybdenum, zinc, chlorine, sodium, cobalt, and silicon. Examples of micronutrients include iron, manganese, zinc, copper, boron, silicon, and molybdenum. The concentration of each micronutrient including both added micronutrients and existing micronutrients from the plant-based protein source, in the biostimulant composition may be about 1% to 15%. Macronutrients include nitrogen, phosphorous, potassium, and calcium. The concentration of each macronutrient including both added macronutrients and existing macronutrients from the plant-based protein source, in the biostimulant composition may be about 1% to 15%, or about 5%.

In specific examples in this paragraph, nitrogen content is from the raw feedstock; no additional nitrogen is added to form the biostimulant composition. In one example, a biostimulant composition has about 5% boron and about 5% nitrogen. In one example, a biostimulant composition has about 5% manganese and about 2% nitrogen. In one example, a biostimulant composition has about 14% potassium and about 1% nitrogen. In one example, a biostimulant composition has about 6% calcium and about 2% nitrogen. In one example, a biostimulant composition has about 4% zinc, about 4% manganese, and about 2% nitrogen. In one example, a biostimulant composition has about 4% zinc and about 3% nitrogen. In some embodiments, nitrogen in these mixtures is from the raw starting material and is not separately added to the composition.

In various embodiments, the biostimulant composition also includes water. In various embodiments, the amount of water in the biostimulant composition is about 1% to 99%. In various embodiments, biostimulant compositions having any of the above concentrations of components may be diluted in water, such as about 40% water. Dilution of a biostimulant composition may result in a particular ratio of non-water components to water. In some embodiments, dilution or evaporation is performed to obtain a density of about 1 gr/ml to 3 gr/ml, or about 1.1 gr/ml or about 1.3 gr/ml. In some embodiments, the biostimulant composition is diluted in water such that concentrations of amino acids present in the biostimulant composition are divided in half.

In various embodiments, the free amino acid component of the biostimulant composition includes about 40 (w/w) % to 60 (w/w) % lysine, in which (w/w) % indicates the weight of the target amino acid versus the weight of the total weight of free amino acids in the biostimulant composition, and about 1 wt % to 15 wt % boron. In various embodiments, the free amino acid component of the biostimulant composition includes about 40 (w/w) % to 60 (w/w) % lysine, and about 1 wt % to 15 wt % manganese. In various embodiments, the free amino acid component of the biostimulant composition includes about 40 (w/w) % to 60 (w/w) % lysine, and about 1 wt % to 15 wt % zinc. In various embodiments, the free amino acid component of the biostimulant composition includes about 40 (w/w) % to 60 (w/w) % lysine, and about 1 wt % to 15 wt % calcium. In various embodiments, the free amino acid component of the biostimulant composition includes about 40 (w/w) % to 60 (w/w) % lysine, and about 1 wt % to 15 wt % manganese. In various embodiments, the free amino acid component of the biostimulant composition includes about 40 (w/w) % to 60 (w/w) % lysine, about 1 wt % to 15 wt % manganese, and about 1 wt % to 15 wt % zinc.

In various embodiments, the free amino acid component of the biostimulant composition includes about 40 (w/w) % to 60 (w/w) % lysine and about 30 (w/w) % to 40 (w/w) % glutamic acid and glutamine. In various embodiments, the free amino acid component of the biostimulant composition includes about 40 (w/w) % to 60 (w/w) % lysine, about 30 (w/w) % to 40 (w/w) % glutamic acid and glutamine, and about 1 wt % to 15 wt % manganese, zinc, or calcium. In various embodiments, the free amino acid component of the biostimulant composition includes about 40 (w/w) % to 60 (w/w) % lysine about 30 (w/w) % to 40 (w/w) % glutamic acid and glutamine, about 1 wt % to 15 wt % manganese, and about 1 wt % to 15 wt % zinc.

In one example, a 1 Liter (L) biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 14% added water-soluble potassium including potassium that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble boron including boron that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 6% added water-soluble calcium including calcium that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble manganese including manganese that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble magnesium (e.g., MgO) including magnesium that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble zinc including zinc that may have been from the plant-based protein source, and has about 10% free amino acids of the total 1 L of biostimulant.

In one example, a 1 L biostimulant includes 550 ml of water and 450 ml of biostimulant composition (e.g., amino acids, oligopeptides, phytohormones, micronutrients, macronutrients, and other components derived from the plant-based protein source) before added micronutrients and/or macronutrients, includes about 4% added water-soluble zinc including zinc that may have been from the plant-based protein source, and about 4% added water-soluble manganese including manganese and has about 10% free amino acids of the total 1 L of biostimulant.

Biostimulant compositions described herein may be packaged in liquid form of bottles of various sizes, including but not limited to 1 L bottles, 5 L bottles, 20 L bottles, and 1000 L bottles.

### Further Additives for Formulations

Such formulations can include further additives, such as metabolic inhibitors, stabilizers, nutrients, biostimulants, and the like. In one embodiment, the additive is a secondary metabolite, such as an antibiotic. In yet another embodiment, the additive is another active ingredient, such as a fungicide, an herbicide, a biosanitizer product, or a fertilizer. The type of additive(s) (e.g., fungicides, herbicides, or others) and concentration of additive(s) can be selected so that they do not negatively affect the activity or viability of the protease(s).

Non-limiting metabolic inhibitors include copper sulfate. Use of such metabolic inhibitors may increase effectiveness as a fungicide.

Non-limiting stabilizers can include polysaccharides, such as cellulose; saccharides, including monosaccharides and disaccharides; sugar alcohols (e.g., mannitol or sorbitol); polyols (e.g., any described herein); starches, such as potato starch; clays, such as kaolin, and the like; organic acids, such as lactic acid, as well as combinations thereof. Use of such stabilizers may increase longevity of the spores and/or mycelium in formulations.

Non-limiting nutrients can include micronutrients, such as boron, chlorine, copper, iron, manganese, molybdenum, and zinc; macronutrients, such as calcium, magnesium, nitrogen, phosphorous, potassium, sulfur, sodium, and silicon; phosphates; nitrates (e.g., ammonium nitrate); sulfates; urea; and combinations of any of these.

Biostimulants can include any compound that can promote growth of the Trichoderma species and/or the plant. Non-limiting biostimulants can include nutrients or micronutrients (e.g., any described herein), lignosulphonates, organic acids, amino acids, carotenoids, peptides, proteins, or proteases. Non-limiting enzymes can include proteases, and the like.

Additives can be provided within the primary formulation including the protease(s) from the Trichoderma species. For instance, such additives can include those that stabilize the formulation (e.g., for a longer shelf life at refrigerated and/or room temperature storage), increase the stability of the protease, etc. In yet other embodiments, the formulation is stored under refrigeration (e.g., about 4° C.), thereby increasing shelf life while maintaining reproducibility and viability of the protease(s).

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

SEQUENCES

GenBank Accession No.: AJ251698, Trichoderma reesei internal transcribed spacer 1 (ITS1),
isolate 6 (SEQ ID NO: 110):

| | |
|---|---|
| 1 | ccgagtttac aactcccaaa ccccaatgtg aacgttacca atctgttgcc tcggcgggat |
| 61 | tctctgcccc gggcgcgtcg cagccccgga tcccatggcg cccgccggag gaccaactca |
| 121 | aactcttttt tctctccgtc gcggcttccg tcgcggctct gttttacctt tgctctgagc |
| 181 | ctttctcggc gaccctagcg ggcgtctcga aaatgaatca |

GenBank Accession No.: AJ563621, Hypocrea jecorina partial tef1 gene for translation
elongation factor 1, exons 5-6, strain IMI 113135 (SEQ ID NO: 111):

| | |
|---|---|
| 1 | cccaagtact atgtcaccgt cattggtatg ttggcagcca acatctcatt gcgtcgttga |
| 61 | cacgtcaaac taacgatgcc ctcacagacg ctcccggcca ccgtgacttc atcaagaaca |
| 121 | tgatcactgg tacttcccag g |

GenBank Accession No.: KF699130, Trichoderma parareesei strain T6 translation elongation
factor 1-alpha (tef1) gene, partial cds (SEQ ID NO: 112):

| | |
|---|---|
| 1 | attctccctt gcctatctgt ccaacatttg tcgaccaaat gttgtgccga caggtttttt |
| 61 | tcatcacccc gctttcttct acccctccga gcgacgcaaa tttttttgct gccttacgat |
| 121 | gggttttagt ggggttgcat cgagcaaccc caccaatact ctggccgctc tgtcggatcc |
| 181 | ttcgacaaca gtcacctcag cacacgcgtc accaacacag cagtctttga tccgcgatgc |
| 241 | taaccatgtt cccctccata ggaagccgcc gaactcggca agggttcctt caagtacgcg |
| 301 | tgggttcttg acaagctcaa ggccgagcgt gagcgtggta tcaccatcga cattgccctc |
| 361 | tggaagttcg agactcccaa gtactatgtc accgtcattg gtatgttggc agccaacatc |
| 421 | tcattgcgtc gttgacacgt caaactaacg atgccctcac agacgctccc ggccaccgtg |
| 481 | acttcatcaa gaacatgatc actggtactt cccaggccga ctgcgctatc ctcattatcg |
| 541 | ctgccggtac tggtgagttc gaggctggta tctccaagga tggccagacc cgtgagcacg |
| 601 | ctctgctcgc ctacaccctg ggtgtcaagc agctcatcgt cgccatcaac aagatggaca |
| 661 | ctgccaactg ggccgaggct cgttaccagg aaatcatcaa ggagacttcc aacttcatca |
| 721 | agaaggtcgg cttcaacccc aaggccgttg ctttc |

GenBank Accession No.: KF699131, Trichoderma parareesei strain T6 calmodulin (CAL1)
gene, partial cds (SEQ ID NO: 113):

| | |
|---|---|
| 1 | ggggggttgt ttacagggtg ctgaccgagc tgctctccag gacaaggacg gcgatggtac |
| 61 | gtgatggcga gtgacgcgac aacacactta ttgccctctc tacgaagccg caccgaagca |
| 121 | ctttttgccg atcgatcact ctctcgtcga ctcgaatcat gatacatgga caagaaactg |
| 181 | acaggcttga cctcgtaggc cagatcacca ccaaggagct gggcaccgtg atgcgctctc |
| 241 | tcggccagaa cccttccgag tcggagctgc aggacatgat caacgaggtt gacgccgaca |
| 301 | acaacggttc catcgacttc cctggtacgt gaattgttgg gagatttggt ggttgaggta |
| 361 | cacgggctga cgtggagcgg tgaagaattt ctcacca |

Primer EF1-728F for tef1 (SEQ ID NO: 114):
5′-CATCGAGAAGTTCGAGAAGG-3′
Primer TEF1-LLErev for tef1 (SEQ ID NO: 115):
5′-AACTTGCAGGCAATGTGG-3′
Primer CAL-228F for call (SEQ ID NO: 116):
5′-GAGTTCAAGGAGGCCTTCTCCC-3′
Primer CAL-737R for call (SEQ ID NO: 117):
5′-CATCTTTCTGGCCATCATGG-3′
GenBank Accession No.: AJ517317, Trichoderma virens internal transcribed spacer 1 (ITS1)
(SEQ ID NO: 120):

| | |
|---|---|
| 1 | ccgagtttac aactcccaaa cccaatgtga acgttaccaa actgttgcct cggcgggatc |
| 61 | tctgccccgg gtgcgtcgca gccccggacc aaggcgcccg ccggaggacc aaccaaaact |
| 121 | cttattgtat accccctcgc gggtttttta ctatctgagc catctcggcg cccctcgtgg |

GenBank Accession No.: FJ788527.1, Hypocrea virens strain T59 thioredoxin-like protein
(Dim1) gene, complete cds (SEQ ID NO: 121):

| | |
|---|---|
| 1 | atgggctctg tcgttctccc gcatctaaac tcaggctggc acgtcgacca ggccatttta |
| 61 | tcgggttcgt cactctcctt gccgttgctc catcttattt atcgtgacat gtgatgcagt |
| 121 | gtgtgctgac cagttgcata gaagaggatc gtctcgtcgt catccggttc ggacgtgacc |
| 181 | acgatcgggt aagaagagtc acttttactc ttggatattt tcccttacta actgccatta |
| 241 | ggactgcatg ctgcaagatg aagtgctcta caagatagcc gatcgcgtca aaaactttgc |
| 301 | cgtcatttac ctctgcgaca ttgacgaggt aggtttttca tctcatccat ggagtaaagg |
| 361 | ccgcttttaa ctggaagtag gttcctgatt tcaacgccat gtacgaactg tacgaccctt |
| 421 | gctctattct gttcttcttt cgcaacaagc atatgatgtg cgattttggt accggtaaca |
| 481 | acaacaagct taactgggtg ctggaggata agcaagagct cattgacatt attgaaacga |
| 541 | tttaccgcgg agcaaagaaa ggtagaggtt tggtggtcag ccccaaggat tacagcacga |
| 601 | gacacagata ctag |

GenBank Accession No.: ACY01406.1, thioredoxin-like protein [Trichoderma virens], amino
acids 1-143 (SEQ ID NO: 122):

| | |
|---|---|
| 1 | MGSVVLPHLN SGWHVDQAIL SEEDRLVVIR FGRDHDRDCM LQDEVLYKIA DRVKNFAVIY |
| 61 | LCDIDEVPDF NAMYELYDPC SILFFFRNKH MMCDFGTGNN NKLNWVLEDK QELIDIIETI |
| 121 | YRGAKKGRGL VVSPKDYSTR HRY |

GenBank Accession No.: ACY01406.1, thioredoxin-like protein [Trichoderma virens], amino
acids 6-137 (SEQ ID NO: 123):

| | |
|---|---|
| 1 | LPHLNSGWHV DQAILSEEDR LVVIRFGRDH DRDCMLQDEV LYKIADRVKN FAVIYLCDID |
| 61 | EVPDFNAMYE LYDPCSILFF FRNKHMMCDF GTGNNNKLNW VLEDKQELID IIETIYRGAK |
| 121 | KGRGLVVSPK DY |

Primer TRX-5 for dim1 (SEQ ID NO: 124):
5′-GAAGAGGATCGTCTCGTCGTC-3′
Primer TRX-3 for dim1 (SEQ ID NO: 125):
5′-TCAGGAACCTCGTCAATGTCG-3′
GenBank Accession No.: AJ224008, Trichoderma harzianum 5.8S rRNA and ITS1 and ITS2
DNA, isolate 11 (SEQ ID NO: 130):

| | |
|---|---|
| 1 | agggatcatt accgagttta caactcccaa acccaatgtg aaccatacca aactgttgcc |
| 61 | tcggcggggt cacgccccgg gtgcgtcgca gccccggaac caggcgcccg ccggagggac |
| 121 | caaccaaact cttttctgta gtcccctcgc ggacgttatt tcttacagct ctgagcaaaa |
| 181 | attcaaaatg aatcaaaact ttcaacaacg gatctcttgg ttctggcatc gatgaagaac |
| 241 | gcagcgaaat gcgataagta atgtgaattg cagaattcag tgaatcatcg aatctttgaa |
| 301 | cgcacattgc gcccgccagt attctggcgg gcatgcctgt ccgagcgtca tttcaaccct |
| 361 | cgaacccctc cggggggtcg gcgttgggga cctcgggagc ccctaagacg ggatcccggc |
| 421 | cccgaaatac agtggcggtc tcgccgcagc ctctcctgcg cagtagtttg cacaactcgc |
| 481 | accgggagcg cggcgcgtcc acgtccgtaa aacacccaac ttctgaaatg ttgacctcgg |
| 541 | atcaggtagg aatacccgct gaacttaa |

GenBank Accession No.: AJ563609, Trichoderma cf. viride partial tef1 gene for translation
elongation factor 1, exons 5-6, strain IMI 352941 (SEQ ID NO: 131):

| | |
|---|---|
| 1 | cccaagtact atgtcaccgt cattggtatg ttttcgcttt tcctcattga tacttggaga |
| 61 | ccaagattct aacgtgccgc tctgtagacg ctcccggtca ccgtgatttc atcaagaaca |
| 121 | tgatcactgg tacttcccag g |

GenBank Accession No.: AJ223773, Trichoderma viride 5.8S rRNA gene, ITS1 and ITS2,
isolate 25 (SEQ ID NO: 140):

| | |
|---|---|
| 1 | agggatcatt accgagttta caactcccaa acccaatgtg aacgttacca aactgttgcc |
| 61 | tcggcggggt cacgccccgg gtgcgtcgca gccccggaac caggcgcccg ccggaggaac |
| 121 | caaccaaact ctttctgtag tcccctcgcg gacgtatttc tttacagctc tgagcaaaaa |
| 181 | ttcaaaatga atcaaaactt tcaacaacgg atctcttggt tctggcatcg atgaagaacg |
| 241 | cagcgaaatg cgataagtaa tgtgaattgc agaattcagt gaatcatcga atctttgaac |
| 301 | gcacattgcg cccgccagta ttctggcggg catgcctgtc cgagcgtcat ttcaaccctc |
| 361 | gaacccctcc gggggatcgg cgttggggat cgggacccct cacacgggtg ccggccccta |
| 421 | aatacagtgg cggtctcgcc gcagcctctc ctgcgcagta gtttgcacaa ctcgcaccgg |
| 481 | gagcgcggcg cgtccacgtc cgtaaaacac ccaactttct gaaatgttga cctcggatca |
| 541 | ggtaggaata cccgctgaac ttaa |

GenBank Accession No.: AJ563611, Trichoderma asperellum partial tef1 gene for translation
elongation factor 1, exons 5-6, strain IMI 296237 (SEQ ID NO: 141):

| | |
|---|---|
| 1 | cccaagtact atgtcaccgt cattggtatg ttttggactc ttctctctag ctatcgacat |
| 61 | tccaagtccg ccattctaac atgctcttcc cacagacgct cccggtcacc gtgatttcat |
| 121 | caagaacatg atcactggta cctcccagg |

5′ primer binding to the 3′ end of the 18S rDNA gene and in proximity to the 5′ end of
the ITS1 region (ITS1 primer, SEQ ID NO: 142):
5′-TCCGTAGGTGAACCTGCGG-3′
3′ primer binding to the 5′ end of the 5.8S rDNA gene and in proximity to the 3′ end of
the ITS1 region (ITS2 primer, SEQ ID NO: 143):
5′-GCTGCGTTCTTCATCGATGC-3′
3′ primer binding to the 5′ end of the 28S rDNA gene and in proximity to the 3′ end of
the ITS2 region (ITS4 primer, SEQ ID NO: 144):
5′-TCCTCCGCTTATTGATATGC-3′
5′ primer binding to the 5′ end of the 5.8S rDNA gene (ITS3 primer, SEQ ID NO: 145):
GCATCGATGAAGAACGCAGC-3′
Primer tef1fw for tef1 (SEQ ID NO: 146):
5′-GTGAGCGTGGTATCACCA-3′
Primer tef1rev for tef1 (SEQ ID NO: 147):
5′-GCCATCCTTGGAGACCAGC-3′
GenBank Accession No.: AF278790, Trichoderma harzianum strain CECT 2413 internal trans-
cribed spacer 1, partial sequence; 5.8S ribosomal RNA gene and internal transcribed
spacer 2, complete sequence; and 28S ribosomal RNA gene, partial sequence (SEQ ID NO: 150):

| | |
|---|---|
| 1 | accgaattta caactcccaa acccaatgtg aacgttacca aagtgttgcc tcggcgggat |
| 61 | ctctgacccc gggtgcgtcg cagccccgga ccaaggcgcc cgccgganga ccaaccaaaa |
| 121 | ctcttattgt ataccccctc gcgggttttt tttataatct gagccttctc ggcgcctctc |
| 181 | gtaggcgttt cgaaaatgaa tcaaaacttt caacaacgga tctcttggtt ctggcatcga |
| 241 | tgaagaacgc agcgaaatgc gataagtaat gtgaattgca gaattcagtg aatcatcgaa |
| 301 | tctttgaacg cacattgcgc ccgccagtat tctggcgggc atgcctgtcc gagcgtcatt |
| 361 | tcaaccctcg aacccctccg gggggtcggc gttggggatc ggccctgcct tggcggtggc |
| 421 | cgtttccgaa atacagtggc ggtttcgccg cagcctttcc tgcgcagtag tttgcacact |
| 481 | cgcatcggga gcgcggcgcg tccacagccg ttaaacaccc aacttctgaa atgttgacct |
| 541 | cggat |

GenBank Accession No.: FJ545255, Hypocrea lixii strain ATCC 20847 18S ribosomal RNA gene,
partial sequence; internal transcribed spacer 1, 5.8S ribosomal RNA gene, and internal
transcribed spacer 2, complete sequence; and 28S ribosomal RNA gene, partial sequence (SEQ
ID NO: 151):

| | |
|---|---|
| 1 | gcggagggat cattaccgag tttacaactc ccaaacccaa tgtgaacgtt accaaactgt |
| 61 | tgcctcggcg ggatctctgc cccgggtgcg tcgcagcccc ggaccaaggc gcccgccgga |
| 121 | ggaccaacca aaactcttat tgtatacccc ctcgcgggtt tttttataat ctgagccttc |
| 181 | tcggcgcctc tcgtaggcgt ttcgaaaatg aatcaaaact ttcaacaacg gatctcttgg |
| 241 | ttctggcatc gatgaagaac gcagcgaaat gcgataagta atgtgaattg cagaattcag |
| 301 | tgaatcatcg aatctttgaa cgcacattgc gcccgccagt ttctggcgg gcatgcctgt |
| 361 | ccgagcgtca tttcaaccct cgaacccctc cggggggtcg gcgttgggga tcggccctgc |
| 421 | cttggcggtg gccgtctccg aaatacagtg gcggtctcgc cgcagcctct cctgcgcagt |
| 481 | agtttgcaca ctcgcatcgg gagcgcggcg cgtccacagc cgttaaacac ccaacttctg |
| 541 | aaatgttgac ctcggatcag gtaggaatac ccgctgaact taagcatatc a |

GenBank Accession No.: KU933430, Trichoderma afroharzianum strain ATCC 20847
translation elongation factor 1-alpha (EF1a) gene, partial cds (SEQ ID NO: 152):

| | |
|---|---|
| 1 | cactggtact tcccaggccg attgcgctat cctcatcatt gccgccggta ctggtgagtt |
| 61 | cgaggctggt atctccaagg atggccagac ccgtgagcac gctctgctcg cctacaccct |
| 121 | gggtgttaag cagctcatcg ttgccatcaa caagatggac actgccaact gggccgaggc |
| 181 | tcgttaccag gaaatcatca aggagacttc caacttcatc aagaaggtcg gcttcaaccc |
| 241 | caaggctgtt gctttcgtcc ccatctccgg tttcaacggt gacaacatgc tccagccctc |
| 301 | caccaactgc ccctggtaca agggctggga gaaggagacc aaggctggca agttcaccgg |
| 361 | caagaccctc cttgaggcca tcgactccat cgagcccccc aagcgtccca cggacaagcc |
| 421 | cctccgtctt cccctccagg atgtctacaa gatcggtggt attggaacag ttcccgtcgg |
| 481 | ccgtatcgag actggtgtcc tcaagcccgg tatggtcgtc actttcgctc cctccaacgt |
| 541 | caccactgaa gtcaagtccg tcgagatgca ccacgagcag ctcgtcgagg gtgttcccgg |
| 601 | tgacaacgtt ggtttcaacg tcaagaacgt ttccgttaag gaaattcgcc gtggtaacgt |
| 661 | tgccggtgac tccaagaacg acccccccat gggtgccgct tctttcaccg ctcaggtcat |
| 721 | cgtcatgaac caccctggcc aggtcggtgc cggctacgcc cccgttcttg actgccacac |
| 781 | tgcccacatt gcctgcaagt tcgccgagct ccaggagaag atcgaccgcc gtaccggtaa |
| 841 | ggctaccgag actgccccca agttcatcaa gtccggtgac tctgccatcg tcaagatgat |
| 901 | tccctccaag cccatgtgcg ttgaggcttt caccgactac cctcccctgg gtcgtttcgc |
| 961 | cgtccgtg |

GenBank Accession No.: AJ251702, Trichoderma longibrachiatum internal transcribed spacer 1
(ITS1), isolate F724 (SEQ ID NO: 153):

| | |
|---|---|
| 1 | ccgagtttac aactcccaaa cccaatgtga acgttaccaa tctgttgcct cggcgggatt |
| 61 | ctcttgcccc gggcgcgtcg cagccccgga tcccatggcg cccgccggag gaccaactcc |
| 121 | aaactctttt tttctctccg tcgcggctcc cgtcgcggct ctgttttatt tttgctctga |
| 181 | gcctttctcg gcgaccctag cgggcgtctc gaaaatgaat ca |

1. A protease formulation comprising:
one or more proteases from a Trichoderma species; and
one or carriers and/or additives, wherein the formulation is configured to hydrolyze a plant protein or a feedstock.

2. The protease formulation of claim 1, wherein the protease is an alkaline proteinase, a serine protease, a subtilisin-like protease, a trypsin-like serine protease, an aspartyl protease, a metallopeptidase, a carboxypeptidase, a glutamate/glutamine protease, or a cysteine protease.

3. The protease formulation of claim 1, wherein the protease comprises a polypeptide sequence having at least 90% sequence identity to any one of the following SEQ ID NOs:1-18, 20-30, 40-51, 60-73, 80-87, 90-94, and 100-107.

4. The protease formulation of claim 1, wherein the protease comprises a polypeptide sequence having at least 90% sequence identity to any one of the proteins associated with the UniProtKB Entry numbers in Tables 1-5.

5. The protease formulation of claim 1, wherein the Trichoderma species is T. parareesei, T. virens, T. atroviride, and/or T. asperellum.

6. The protease formulation of claim 5, wherein the Trichoderma species is T. parareesei strain T6, T. virens strain T59, T. atroviride strain T11, or T. asperellum strain T25.

7. The protease formulation of claim 1, wherein the one or more carriers comprises a liquid carrier or a solid carrier.

8. The protease formulation of claim 1, wherein the one or more additives comprises a metabolic inhibitor, a stabilizer, and/or a nutrient.

9. A method of preparing a hydrolyzed composition, the method comprising:
receiving a plant-based feedstock including a plant protein; and
hydrolyzing the plant protein with a protease to produce a hydrolysis product comprising an amino acid and/or an oligopeptide, wherein the protease is from a Trichoderma species.

10. The method of claim 9, wherein the hydrolysis product is a biofungicide composition.

11. The method of claim 9, wherein the plant-based feedstock is selected from the group consisting of legumes, tarwi, peanut, carob germ, soybean, and Plukenetia volubilis.

12. The method of claim 9, wherein said hydrolyzing comprises introducing a protease formulation of any one of claims 1-8 to the plant-based feedstock.

13. The method of claim 9, wherein said hydrolyzing comprises introducing an isolate comprising the Trichoderma species to the plant protein.

14. The method of claim 9, wherein said hydrolyzing comprises introducing the protease to the plant-based feedstock.

15. The method of claim 9, wherein the protease is an alkaline proteinase, a serine protease, a subtilisin-like protease, a trypsin-like serine protease, an aspartyl protease, a metallopeptidase, a carboxypeptidase, a glutamate/glutamine protease, or a cysteine protease.

16. The method of claim 9, wherein the protease comprises a polypeptide sequence having at least 90% sequence identity to any one of the following SEQ ID NOs:1-18, 20-30, 40-51, 60-73, 80-87, 90-94, and 100-107.

17. The method of claim 9, wherein the protease comprises a polypeptide sequence having at least 90% sequence identity to any one of the proteins associated with the UniProtKB Entry numbers in Tables 1-5.

18. The method of claim 9, wherein the Trichoderma species is T. parareesei, T. virens, T. atroviride, and/or T. asperellum.

19. The method of claim 18, wherein the Trichoderma species is T. parareesei strain T6, T. virens strain T59, T. atroviride strain T11, or T. asperellum strain T25.

20. The method of claim 9, wherein the amino acid is selected from the group consisting of glutamic acid, glutamine, glycine, threonine, alanine, leucine, lysine, and combinations thereof.

21. The method of claim 9, wherein the amino acid is present in a trace amount, and wherein the amino acid is selected from the group consisting of aspartic acid, serine, tyrosine, arginine, valine, tryptophan, phenylalanine, asparagine, isoleucine, histidine, methionine, glutamine, proline, hydroxyproline, ornithine, taurine, and combinations thereof.

22. The method of claim 9, wherein the hydrolysis product further comprises one or more phytohormones.

23. The method of claim 22, wherein the one or more phytohormones are selected from the group consisting of cytokinins, abscisic acids (ABAs), jasmonates, auxins, and phenolics.

24. The method of claim 9, further comprising:
adding one or more nutrients to the plant-based feedstock or the hydrolysis product, thereby providing a biostimulant formulation.

25. The method of claim 24, wherein the one or more nutrients are selected from the group consisting of calcium, potassium, sulfur, magnesium, carbon, oxygen, hydrogen, iron, manganese, boron, molybdenum, zinc, chlorine, sodium, and cobalt.

26. A biostimulant composition or a biofungicidal composition comprising:
a hydrolysis product comprising an amino acid and an oligopeptide, wherein the amino acid and the oligopeptide are derived from a plant-based feedstock; and
a trace amount of a protease, or fragments thereof, from a Trichoderma species.

27-35. (canceled)

36. A biostimulant formulation or a biofungicidal formulation comprising:
a biostimulant composition or a biofungicidal composition of claim 26; and
one or more nutrients comprising a micronutrient and/or a macronutrient.

37-38. (canceled)

39. A method of treating a plant, the method comprising:
preparing a composition of claim 26 and
delivering the composition to the plant, a portion thereof, a plant material, or a soil in proximity to the plant.

40-42. (canceled)


SEQUENCE LISTING (166 sequences)

SEQ ID NO: 1 - protein, 409 aa, unidentified
  source 1..409: note = Hypocrea atroviridis
MTSIRRLALYLGALLPAVLAAPAALHKKPEAVPNKFIVTLKEGASIDTDSHLAWVTDIHTRSLTKRSTAGVEKTYNIHTWNAYAGEFDEETIEQIKSNPDVASVEPDYIMHLSDIVEDKRALTTQSGAPWGLGTVSHRTSGSTSYIYDSSAGAGTFAYVVDSGINTSHQQFGGRASLGYNAAGGQHVDTLGHGTHVSGTIGGSTYGVAKQASLISVKVFAGESASTSVILDGYNWAVNDIVSKSRASKSAINMSLGGPASSTWTTAINAAFNQGVLTIVAAGNGDSLGNPQPVSGTSPANVPNAITVAALDINWRTASFTNYGAGVDVFAPGVNILSSWIGSNTATNTISGTSMATPHVVGLALYLQSLEGLTSPTAVTNRIKALATSGRVTGSLNGSPNVIIFNGNSS

SEQ ID NO: 2 - protein, 409 aa, unidentified
  source 1..409: note = T. virens
MASIRRLALYLGALLPAVLAAPAGHHKAPEAVPNKFIVTLKEGASIDTDSHLTWVNDLHRRSLTKRNTAGVEKTYNIHTWSAYAGEFDEDTIEQIKASPDVASVEPDYIMYLSDIVEDKRALTTQTGAPWGLGTVSHRTSGSTSYIYDTSAGSGTFAYVVDSGINTSHQQFGGRASLGYNAAGGQHVDTLGHGTHVSGTIGGSTYGVAKQASLISVKVFQGNSASTSVILDGYNWAVNDIVSRNRASKSAINMSLGGPASSAWTTAINAAFNQGVLTIVAAGNGDSLGNPQPVSGTSPANVPNAITVAALDINWRTASFTNYGAGVDVFAPGVNILSSWIGSNTATNTISGTSMATPHVVGLALYLQSLEGLATPTAVTNSPNRIKALATSGRVTGSLNGVLIFNGNSA

SEQ ID NO: 3 - protein, 409 aa, unidentified
  source 1..409: note = T. hamatum
MTSIRRLALYLGALLPAVLAAPAALHKKPEAAPNKFIVTLKEGASIDTDSHLAWVNDIHRRSLTKRSTAGVEKTYNIHTWSAYAGEFDAETIEQIKSSPDVASVEPDYIMYLSDIVEDKRALTTQSGAPWGLGTVSHRTSGSTSYIYDSSAGSGTFAYVVDSGINTSHQQFGGRASLGYNAAGGQHVDTLGHGTHVSGTIGGSTYGVAKQASLISVKVFAGESATTSVILDGYNWAVNDIVSRNRASKSAINMSLGGPASSTWTTAINAAFNQGVLTIVAAGNGDSFGNPQPVSGTSPANVPNAITVAALDINWRTASFTNYGAGVDVFAPGVNILSSWIGSNTATNTISGTSMATPHVVGLALYLQSLEGLTTPTAVTNRIKALATAGRITGSLNGSPNTLIFNGNSA

SEQ ID NO: 4 - protein, 408 aa, unidentified
  source 1..408: note = T. harzianum
MAWFKTLALFLFTVTPYVTALPLKTTGQGSVLDVDGLADEDWYIVTLHKNISPSAFAMHLAKINDTYQHNSLNDEQNRPSGVGKTFAIGDYRGYSGVFDKDTIALLWKDADVAEIERDEFCTIQGKLVTQRHAPWGLAALSSKKPGAKSYRYDKSAGNGTFAYVVDSGVFTKHDEFGGRASVRYSVDPSDLSDVEGHGTHVAGIIAGKTFGVAKQAEIIGVKVLPDSVGIISDVLDGFQWAVQDIILKGRQNRSVINLSIASPFSIAFNRAVDAAFEMGILSVVAAGNAGMEARKMSPVSAAEALAVGAINSEWTQTIFSNFGPEVNIQAPGYHIESAYIGTENATNIMSGTSMSAPHVAGLALYLAALENTKNAKELRDRILQLGVEGKAKELTSDTPNLVAHNGAR

SEQ ID NO: 5 - protein, 415 aa, unidentified
  source 1..415: note = T. gamsii
MVLFKHLALLLMAALPRIAALPVNDVNAINVNDVNINENSISAEGRYIVTLQNNMPMPEVRNHMSRVAAIQYRNSGNHRFPHTGIEKSYAIGEFRAYVGAFDQDTLQMIRNDTKVAEIEPDTVVRLHDSTSYKAASQPNAPWGLATLSSKRRNTGTYHYDKSAGNGTFAYVLDTGINVNHVDFEGRASYGYNAVDGEFTDLQGHGTHCAGIIGSKSFGVAKQAQLIAVKTFRNHETLMSTVMDAYQWSANDIRTKNRVGLAVISMSLGGAYSRAMNHAVESVFDMGILTVVAAGNEGKPTRQFSPASAANAFTVGAIDADWHYWEHSNFGPEVNIFAPGVGIESTYISTDMATRKLSGTSMAAPHVAGLALYLCALEKIDNPTALRERILALGTRGRIHKLKGESPNLIAHNNLH

SEQ ID NO: 6 - protein, 409 aa, unidentified
  source 1..409: note = T. guizhouense
MASIRRLALYLGALLPAVLAAPAAHHKAPEVVPNKFIVTLKEGASVDTDSHLTWVNDLHRRSLTKRHTAGVEKTYNIHTWNAYAGEFDAETLEQIKASPDVASVEPDYIMYLSDIVEDKRALTTQTGAPWGLGTVSHRTSGSTSYIYDTSAGSGTYAYVVDSGINTSHQQFGGRATLGYNAAGGQHVDTLGHGTHVSGTIGGSTYGVAKQANLVSVKVFQGNSASTSVILDGYNWAVNDIVSKNRASKSAINMSLGGPSSSTWTTAINAAYNQGVLTIVAAGNGDSLGNPQPVSGTSPANVPNAITVAAIDINWRTASFTNYGAGVDVFAPGVNILSSWIGSNTATNTISGTSMATPHVVGLALYLQSLEGLSTPAAVTNRIKALATSGRVTGSLNGSPNVIIFNGNSA

SEQ ID NO: 7 - protein, 409 aa, unidentified
  source 1..409: note = T. asperellum
MASIRRLALYLGALLPAVLAAPAALHKKPEAVPNKFIVTLKEGASIDTDSHIAWVNDLHRRSLTKRSTAGVEKTYNIHTWSAYAGEFDEETIEQIKSSPDVASVEPDYIMYLSDIVEDKRALTTQSGAPWGLGTVSHRTSGSTSYIYDSSAGAGTFAYVVDSGINTSHQQFGGRASLGYNAAGGQHVDTLGHGTHVSGTIAGSTYGVAKQASLISVKVFSGDSSTTSIILDGYNWAVNDIVSRNRASKSAINMSLGGPASSTWTTAINAAFNQGVLTIVAAGNGDSLGNPQPVSSTSPANVPNAITVAALDINWRTASFTNYGAGVDVFAPGVNVLSSWIGSNTATNTISGTSMATPHVVGLALYLQSLEGLSTPTAVTNRIKALATTGRVTGSLRGSPNSIIFNGNSS

SEQ ID NO: 8 - protein, 408 aa, unidentified
  source 1..408: note = T. arundinaceum
MASLRRLALYLGALLPAVLAAPAAHKAPEAVPNKFIITLKEGASVDTDSHLTWVNDLHRRSLTKRHTAGVEKTYNIHTWNAYAGEFDAETIEQIKASPDVAAVEPDYIMHLTDMLDDKRALTTQTGAPWGLGTVSHRTSGSTSYIYDTSAGAGTFAYVVDSGINTAHQQFGGRASLGYNAAGGQHVDTLGHGTHVSGTIGGSTYGVAKQASLISVKVFSGSTASTSVILDGYNWAVNDIVSRNRASKSAINMSLGGPSSSTWTTAINAAYNQGVLTIVAAGNGDTLGNPLPVSGTSPANVPNAITVAAIDINWRTASFTNYGPGVDVFAPGVNILSSWIGSSTATNTISGTSMATPHVVGLALYLQSLEGLATPAAVTNRIKALATAGRVTGSLNGSPNTIIFNGNSA

SEQ ID NO: 9 - protein, 400 aa, unidentified
  source 1..400: note = T. asperellum
MAWFKQLALFLITALPRIAALPVNDANDNSISAEGRYIVTLQNNIPPSEFRSHMSRVAAIQYRNSGNHKPHVGIERTYAIGEFQAYSGAFDGDTLKMIRNDTRVAEIEPDAVFTLHDKASSQFNAPWGLATLSSKRRNTATYHYDKSAGNGTFAYVVDSGINTKHLEFEGRASNGYNAVDGDFTDLNGHGTHCAGIIGSKSFGVAKQTELIAVKVFLNRETLISNVLDGFQWAVHDIRTKGRQSRAVVNMSFGGNFSRAVNHAVESAFEMGVFTVVAAGNDQKPARDVSPASAPNAFTVGAIDADWHEWEHSNFGPEINIFAPGVEIESTFISTEMATRKLSGTSMAAPHVTGLALYLLALEDLKTPTMLRERILGLGTRNRIHNLKDESPNLIAHNGLH

SEQ ID NO: 10 - protein, 409 aa, unidentified; identical to SEQ ID NO: 1
  source 1..409: note = T. atroviride

SEQ ID NO: 11 - protein, 409 aa, unidentified
  source 1..409: note = T. koningiopsis
MTSIRRLALYLGALLPAVLAAPAALHKKPEAVPNKFIVTLKEGASIDTDSHLAWVNDIHTRSLTKRSTAGVEKTYNIHTWNAYAGEFDEETIEQIKSSPDVASVEPDYIMHLSDIVEDKRALTTQSGAPWGLGTVSHRTSGSTSYIYDSSAGAGTFAYVVDSGINTSHQQFGGRASLGYNAAGGQHVDTLGHGTHVSGTIAGSTYGVAKQASLISVKVFAGESASTSVILDGYNWAVNDIVSRNRASKSAINMSLGGPASSTWTTAINAAFNQGVLTIVAAGNGDSLGNPQPVSGTSPANVPNAITVAALDINWRTASFTNYGAGVDVFAPGVNILSSWIGSNTATNTISGTSMATPHVVGLALYLQSLEGLATPAAVTNRIKALATSGRVTGSLNGSPNVIIFNGNSS

SEQ ID NO: 12 - protein, 409 aa, unidentified
  source 1..409: note = T. reesei
MASLRRLALYLGALLPAVLAAPAVNYKLPEAVPNKFIVTLKDGASVDTDSHLTWVKDLHRRSLGKRSTAGVEKTYNIDSWNAYAGEFDEETVKQIKANPDVASVEPDYIMWLSDIVEDKRALTTQTGAPWGLGTVSHRTPGSTSYIYDTSAGSGTFAYVVDSGINIAHQQFGGRASLGYNAAGGDHVDTLGHGTHVSGTIGGSTYGVAKQASLISVKVFQGNSASTSVILDGYNWAVNDIVSRNRASKSAINMSLGGPASSTWATAINAAFNKGVLTIVAAGNGDALGNPQPVSSTSPANVPNAITVAALDINWRTASFTNYGAGVDVFAPGVNILSSWIGSNTATNTISGTSMATPHVVGLALYLQALEGLSTPTAVTNRIKALATTGRVTGSLNGSPNTLIFNGNSA

SEQ ID NO: 13 - protein, 292 aa, synthetic construct
  VARIANT 1..4: note = X can be any amino acid
  VARIANT 6..7: note = X can be any amino acid
  VARIANT 13..15: note = X can be any amino acid
  VARIANT 17..24: note = X can be any amino acid
  VARIANT 26: note = X can be any amino acid
  VARIANT 29: note = X can be any amino acid
  VARIANT 33: note = X can be any amino acid
  VARIANT 36: note = X can be any amino acid
  VARIANT 40: note = X can be any amino acid
  VARIANT 42: note = X can be any amino acid
  VARIANT 44..47: note = X can be any amino acid
  VARIANT 49..50: note = X can be any amino acid
  VARIANT 52: note = X can be any amino acid
  VARIANT 56..58: note = X can be any amino acid
  VARIANT 60..67: note = X can be any amino acid
  VARIANT 69..70: note = X can be any amino acid
  VARIANT 76..77: note = X can be any amino acid
  VARIANT 79: note = X can be any amino acid
  VARIANT 81..85: note = X can be any amino acid
  VARIANT 91..95: note = X can be any amino acid
  VARIANT 98..106: note = X can be any amino acid
  VARIANT 108..110: note = X can be any amino acid
  VARIANT 112..114: note = X can be any amino acid
  VARIANT 116..118: note = X can be any amino acid
  VARIANT 121..124: note = X can be any amino acid
  VARIANT 126..133: note = X can be any amino acid
  VARIANT 135..139: note = X can be any amino acid
  VARIANT 141..145: note = X can be any amino acid
  VARIANT 147..153: note = X can be any amino acid
  VARIANT 155..158: note = X can be any amino acid
  VARIANT 164..176: note = X can be any amino acid
  VARIANT 179..183: note = X can be any amino acid
  VARIANT 185..186: note = X can be any amino acid
  VARIANT 188: note = X can be any amino acid
  VARIANT 190..193: note = X can be any amino acid
  VARIANT 195..200: note = X can be any amino acid
  VARIANT 202: note = X can be any amino acid
  VARIANT 204..209: note = X can be any amino acid
  VARIANT 213..216: note = X can be any amino acid
  VARIANT 218..219: note = X can be any amino acid
  VARIANT 221..224: note = X can be any amino acid
  VARIANT 227..229: note = X can be any amino acid
  VARIANT 235..236: note = X can be any amino acid
  VARIANT 240: note = X can be any amino acid
  VARIANT 247..248: note = X can be any amino acid
  VARIANT 251..263: note = X can be any amino acid
  VARIANT 266..267: note = X can be any amino acid
  VARIANT 269..281: note = X can be any amino acid
  VARIANT 284..287: note = X can be any amino acid
  VARIANT 289..292: note = X can be any amino acid
XXXXQXXAPWGLXXXSXXXXXXXXYXYDXSAGXGTXAYVXDXGXXXXHXXFXGRAXXXYXXXXXXXXDXXGHGTHXXGXIXXXXXGVAKQXXXXXVKXXXXXXXXXSXXXDXXXWXXXDIXXXXRXXXXXXXXSXXXXXSXXXXXAXXXXXXXGXXXXVAAGNXXXXXXXXXXXXXSPXXXXXAXXVXAXXXXWXXXXXXNXGXXXXXXAPGXXXXSXXIXXXXATXXXSGTSMXXPHVXGLALYLXXLEXXXXXXXXXXXXXRIXXLXXXXXXXXXXXXXPNXXXXNXXXX

SEQ ID NO: 14 - skipped

SEQ ID NO: 15 - protein, 11 aa, synthetic construct
  VARIANT 5..6: note = X can be any amino acid
  VARIANT 10..11: note = X can be any amino acid
HGTHXXGTIXX

SEQ ID NO: 16 - skipped

SEQ ID NO: 17 - protein, 8 aa, synthetic construct
  VARIANT 6..8: note = X can be any amino acid
VAAGNXXX

SEQ ID NO: 18 - protein, 8 aa, synthetic construct
  VARIANT 5..6: note = X can be any amino acid
GTSMXXPH

SEQ ID NO: 19 - skipped

SEQ ID NO: 20 - protein, 878 aa, unidentified
  source 1..878: note = T. harzianum
MVRSAVFLSLLATISGVIARGSGQTSKVVPGAYIFEFENDQDTAAFYKQLNGEGSTRMKFNYKLFKGVSVQLKDLSNHESKAQKMAQLPAVKNMWPVTLIQAPNPKVEWVASSTSPALQARDVSPTDPSNFPPHQMVQIDKLRAKGYTGKGVKVAVIDTGIDYTHPALGGCFGAGCLVSFGTDLVGDNYSGFNTPVPDNDPKDCAGHGSHVAGIIAAQENPYGFTGGAPDVTLGAYRVFGCEGQAGNDVLIAAYNKAFEDGAQIITASIGGPSGWAEEPWAVAVSRIVDAGVPCTVSAGNDGDHGLFFASTAANGKKVLAVASVDNKNIPTVFSLSSYKVDNGDSQQFGYVSSSKAWKGVTKPVYAVTLDTTIPDDACSPLPDSTPDLSNYIVLVRRGTCTFVQKAQNVAAKGAKYLLYYNNIAGALSVDVSTVTQITAAGMVQDSTGAAFIAAIKAGKTVTLSMVDPDSATKQIQFADNKATGGALSTYTSWGPTWEVDVKPQISSPGGNILSTYPVALGGYATLSGTSMACPLTAAAVALVAQARGTFDPALIENLLATTANPQVFNDGAQFYDFLAPVAQQGGGLIQAYDAAFATTLLSPSSLTFNDTDHFVRKRQITLKNTSKQRVTYKLNHVPTNTFYTLSGDSPYPAAFPNEAIAAHASVKLALQTVTLPAGKSITIDVLPTPPKGVDAKRLGLWSGYITVNGTDGSALSVPYQGLTGSLHKQQVLLPEDSWIAKSTDENLNPVENGTVFTIPAPGNAQSTDVLPSLVVSPAFGSRQIRVDLVLLSAPPKNTKIKTTKFLDTTSIGQPAGAPVLWASRGPTPLSWSGELTDNKFAPPGTYKAVFHALRIFGNEKKKEDWDVSESPAFTIKYA

SEQ ID NO: 21 - protein, 878 aa, unidentified
  source 1..878: note = Hypocrea virens
MVRSALFVSLLATISGVIARGSGHPSKVVPGAYIFEFENDQDTADFYKQLNGEGSTRMKFNYKLFKGVSVQLKDLSNHEDKAQKMAQLPAVKNMWPVTLIQAPNPKVEWVASSSSPALQARDVSPTDPSNFPPQQMTQIDKLRAKGFTGKGVKLAVIDTGIDYTHPALGGCFGAGCLVSFGTDLVGDNYSGFNTPVPDNDPMDCAGHGSHVAGIIAAQKNPYGFTGGAPDVTLGAYRVFGCAGQAGNDVLIAAYNKAFEDGAQIITASIGGPSGWAEEPWAVAVSRIVDAGVPCTVSAGNDGDHGLFYASTAANGKKVLAVASVDNANIPTVFSLSSYKVDNGDAQQFGYVSSAKAWKGVNKPIYAVTLDTTIPNDACDPLPDSTPDLSNYIVLVRRGTCTFVQKAQNVAAKGAKYLLYYNTAAGALAVDVSTVTQITAAGMVQDSTGAAWIAALKDGKTVTLSMVNSDEATKQIQFADNKVTGGALSTYTSWGPTWEVDVKPQISSPGGNILSTYPVAKGGYATLSGTSMACPLTAAAVALVGQARKTFDPVLLENLLATTANPQLFNDGAQFYNFLAPVAQQGGGLIQAYDAAFATTLLSPSSLSFNDTDHFVRKRQITLKNTSKQRVTYKLNHVPTNTFYTLTGDSPYPAPFPNDAIAAHATVRLGLEQVTLPAGRSITIDVLPTPPRGVDAKRLGLWSGYITVNGTDGSALSVPYQGLTGSLHKQKVLYPADSWISKSTDENLNPVDNGTVFTIPAPGNAQSTDVLPSLVVSPAFGSRLVRVDLVLLSAPPKNTKVKTTKFLDTTSIGQPAGSPLLWASRGATPLSWSGELTDNKFAPPGTYKAVFHALRIFGNEKKKEDWDVSESPAFTIKYA

SEQ ID NO: 22 - protein, 882 aa, unidentified
  source 1..882: note = Hypocrea jecorina
MVRSALFVSLLATFSGVIARVSGHGSKIVPGAYIFEFEDSQDTADFYKKLNGEGSTRLKFDYKLFKGVSVQLKDLDNHEAKAQQMAQLPAVKNVWPVTLIDAPNPKVEWVAGSTAPTLESRAIKKPPIPNDSSDFPTHQMTQIDKLRAKGYTGKGVRVAVIDTGIDYTHPALGGCFGRGCLVSFGTDLVGDDYTGFNTPVPDDDPVDCAGHGSHVAGIIAAQENPYGFTGGAPDVTLGAYRVFGCDGQAGNDVLISAYNQAFEDGAQIITASIGGPSGWAEEPWAVAVTRIVEAGVPCTVSAGNEGDSGLFFASTAANGKKVIAVASVDNENIPSVLSVASYKIDSGAAQDFGYVSSSKAWDGVSKPLYAVSFDTTIPDDGCSPLPDSTPDLSDYIVLVRRGTCTFVQKAQNVAAKGAKYLLYYNNIPGALAVDVSAVPEIEAVGMVDDKTGATWIAALKDGKTVTLTLTDPIESEKQIQFSDNPTTGGALSGYTTWGPTWELDVKPQISSPGGNILSTYPVALGGYATLSGTSMACPLTAAAVALIGQARGTFDPALIDNLLATTANPQLFNDGEKFYDFLAPVPQQGGGLIQAYDAAFATTLLSPSSLSFNDTDHFIKKKQITLKNTSKQRVTYKLNHVPTNTFYTLAPGNGYPAPFPNDAVAAHANLKFNLQQVTLPAGRSITVDVFPTPPRDVDAKRLALWSGYITVNGTDGTSLSVPYQGLTGSLHKQKVLYPEDSWIADSTDESLAPVENGTVFTIPAPGNAGPDDKLPSLVVSPALGSRYVRVDLVLLSAPPHGTKLKTVKFLDTTSIGQPAGSPLLWISRGANPIAWTGELSDNKFAPPGTYKAVFHALRIFGNEKKKEDWDVSESPAFTIKYA

SEQ ID NO: 23 - protein, 882 aa, unidentified
  source 1..882: note = T. parareesei
MVRSALFVSLLATFSGVIARGSGHGSKIVPGAYIFEFEDSQDTADFYKKLNGEGSTRLKFDYKLFKGVSVQLKDLDNHEAKAQQMAQLPAVKNVWPVTLIDAPNPKVEWVAGSTAPTLESRAIKKPPIPNDSSDFPTHQMTQIDKLRAKGYTGKGVRVAVIDTGIDYTHPALGGCFGRGCLVSFGTDLVGDDYTGFNTPVPDDDPVDCAGHGSHVAGIIAAQKNPYGFTGGAPDVTLGAYRVFGCDGQAGNDVLISAYNQAFEDGAQIITASIGGPSGWAEEPWAVAVTRIVEAGVPCTVSAGNEGDSGLFFASTAANGKKVIAVASVDNKNIPSVLSVASYKIDSGAAQDFGYVSSSKAWDGVSKPLYAVSFDTTIPDDGCSPLPDSTPDLSDYIVLVRRGTCTFVQKAQNVAAKGAKYLLYYNNIPGALAVDVSAIPEIEAVGMVDDKTGATWVAALKDKKTVTLTLTDPIESEKQIQFADNPTTGGALSGYTTWGPTWELDVKPQISSPGGNILSTYPVALGGYATLSGTSMACPLTAAAVALIGQARGTFDPVLIDNLLATTANPQLFNDGEKFYDFLAPVPQQGGGLIQAYDAAFATTLLSPSSLSFNDTDHFIKKKQITLKNTSKQRVTYKLNHVPTNTFYTLAPGNGYPAPFPNDAVAAHANLKFNLQQVTLPAGRSITVDVFPTPPKDVDAKRLALWSGYITVNGTDGTSLSVPYQGLTGSLHKQKVLYPEDSWIADSTDESLAPVENGTVFTIPAPGNAGPDDKLPSLVVSPALGSRYVRVDLVLLSAPPRGTKLKTVQFLDTTSIGQPAGSPLLWISRGANPIAWTGELSDNKFAPPGTYKAVFHALRIFGNEKKKEDWDVSESPAFTIKYA

SEQ ID NO: 24 - protein, 882 aa, unidentified
  source 1..882: note = T. longibrachiatum
MVRSALFVSLLATFSGVIARGSGHGSKVVPGAYIFEFEVSQDTADFYKKLNGEGSTRLKFDYKLFKGVSVQLKDLDNHEAKAQQMAELPAVKNVWPVTLIDAPNPKVEWVAGSTAPTLESRSAKKPPIPNDSSDFPTHQMTQIDKLRAKGYTGKGVRVAVIDTGIDYTHPALGGCFGRGCLVSFGTDLVGDDYTGFNTPVPDDDPVDCAGHGSHVAGIIAAQKNPYGFTGGAPEVTLGAYRVFGCDGQAGNDVLISAYNQAFEDGAQIITASIGGPSGWAEEPWAAAVTRIVEAGVPCTVSAGNEGDSGLFFASTAANGKKVIAVASVDNENIPSVLSVAAYKIDSGAAQDFGYVSSSKAWDGVSKPLYAVSFDTTIPDDGCSALPDSTPDLSDYIVLVRRGTCTFVQKAQNVAAKGAKYLLYYNNIAGALAVDVSAVPEIEAVGMVDDKTGATWIAALKAGKTVTLTLTDPVESEKQIQFADNPTTGGALSGFTTWGPTWELDVKPQISSPGGNILSTYPVALGGYATLSGTSMACPLTAAAVALIGQARGTFDPALIENLLATTANPQLFNDGEKFYDFLAPVPQQGGGLIQAYDAAFATTLLSPSSLSFNDTDHFVKKRQITLKNTSKQRVTYKLNHVPTNTFYTLAPGNGYPAPFPNDAVAAHANLQFGLQQVTLPAGRSITVDVFPSPPKGVDARRLALWSGYITVNGTDGTSLSVPYQGLTGSLHKQKVLYPEDSWIADSTDESLAPVENGTVFTIPAPGNAGPDDKLPSLVVSPALGSRYVRVDLVLLSAPPHGTKLKTVKFLDTTSIGQPAGSPLLWISRGANPLAWNGELSDNKFAPPGTYKAVFHALRIFGNEKKKEDWDVSESPAFTIKYA

SEQ ID NO: 25 - protein, 863 aa, synthetic construct
  VARIANT 2: note = X can be any amino acid
  VARIANT 5..6: note = X can be any amino acid
  VARIANT 9: note = X can be any amino acid
  VARIANT 20..21: note = X can be any amino acid
  VARIANT 26: note = X can be any amino acid
  VARIANT 30: note = X can be any amino acid
  VARIANT 39: note = X can be any amino acid
  VARIANT 42: note = X can be any amino acid
  VARIANT 57: note = X can be any amino acid
  VARIANT 61: note = X can be any amino acid
  VARIANT 65: note = X can be any amino acid
  VARIANT 68: note = X can be any amino acid
  VARIANT 75: note = X can be any amino acid
  VARIANT 82: note = X can be any amino acid
  VARIANT 93: note = X can be any amino acid
  VARIANT 95..96: note = X can be any amino acid
  VARIANT 98: note = X can be any amino acid
  VARIANT 100..101: note = X can be any amino acid
  VARIANT 103..109: note = X can be any amino acid
  VARIANT 111: note = X can be any amino acid
  VARIANT 113: note = X can be any amino acid
  VARIANT 115: note = X can be any amino acid
  VARIANT 118..119: note = X can be any amino acid
  VARIANT 122: note = X can be any amino acid
  VARIANT 132: note = X can be any amino acid
  VARIANT 138..139: note = X can be any amino acid
  VARIANT 159: note = X can be any amino acid
  VARIANT 173: note = X can be any amino acid
  VARIANT 175: note = X can be any amino acid
  VARIANT 184: note = X can be any amino acid
  VARIANT 187: note = X can be any amino acid
  VARIANT 204: note = X can be any amino acid
  VARIANT 215: note = X can be any amino acid
  VARIANT 227: note = X can be any amino acid
  VARIANT 237: note = X can be any amino acid
  VARIANT 241: note = X can be any amino acid
  VARIANT 267: note = X can be any amino acid
  VARIANT 270: note = X can be any amino acid
  VARIANT 274: note = X can be any amino acid
  VARIANT 286: note = X can be any amino acid
  VARIANT 289: note = X can be any amino acid
  VARIANT 293: note = X can be any amino acid
  VARIANT 304: note = X can be any amino acid
  VARIANT 312: note = X can be any amino acid
  VARIANT 316: note = X can be any amino acid
  VARIANT 318: note = X can be any amino acid
  VARIANT 320..322: note = X can be any amino acid
  VARIANT 325: note = X can be any amino acid
  VARIANT 327: note = X can be any amino acid
  VARIANT 329..330: note = X can be any amino acid
  VARIANT 332: note = X can be any amino acid
  VARIANT 339: note = X can be any amino acid
  VARIANT 343: note = X can be any amino acid
  VARIANT 346: note = X can be any amino acid
  VARIANT 349: note = X can be any amino acid
  VARIANT 353..354: note = X can be any amino acid
  VARIANT 360: note = X can be any amino acid
  VARIANT 362: note = X can be any amino acid
  VARIANT 364..365: note = X can be any amino acid
  VARIANT 375: note = X can be any amino acid
  VARIANT 407..409: note = X can be any amino acid
  VARIANT 413: note = X can be any amino acid
  VARIANT 418..421: note = X can be any amino acid
  VARIANT 423: note = X can be any amino acid
  VARIANT 425: note = X can be any amino acid
  VARIANT 429: note = X can be any amino acid
  VARIANT 431: note = X can be any amino acid
  VARIANT 435..437: note = X can be any amino acid
  VARIANT 440: note = X can be any amino acid
  VARIANT 442..443: note = X can be any amino acid
  VARIANT 449..457: note = X can be any amino acid
  VARIANT 463: note = X can be any amino acid
  VARIANT 466..467: note = X can be any amino acid
  VARIANT 474..475: note = X can be any amino acid
  VARIANT 477: note = X can be any amino acid
  VARIANT 484: note = X can be any amino acid
  VARIANT 505: note = X can be any amino acid
  VARIANT 528..529: note = X can be any amino acid
  VARIANT 533: note = X can be any amino acid
  VARIANT 538: note = X can be any amino acid
  VARIANT 540..541: note = X can be any amino acid
  VARIANT 552: note = X can be any amino acid
  VARIANT 557..558: note = X can be any amino acid
  VARIANT 561: note = X can be any amino acid
  VARIANT 567: note = X can be any amino acid
  VARIANT 592: note = X can be any amino acid
  VARIANT 600..601: note = X can be any amino acid
  VARIANT 603: note = X can be any amino acid
  VARIANT 631..635: note = X can be any amino acid
  VARIANT 639: note = X can be any amino acid
  VARIANT 643: note = X can be any amino acid
  VARIANT 645: note = X can be any amino acid
  VARIANT 650..654: note = X can be any amino acid
  VARIANT 656..657: note = X can be any amino acid
  VARIANT 664: note = X can be any amino acid
  VARIANT 668: note = X can be any amino acid
  VARIANT 671: note = X can be any amino acid
  VARIANT 673: note = X can be any amino acid
  VARIANT 676..677: note = X can be any amino acid
  VARIANT 681: note = X can be any amino acid
  VARIANT 684: note = X can be any amino acid
  VARIANT 698..699: note = X can be any amino acid
  VARIANT 715: note = X can be any amino acid
  VARIANT 718: note = X can be any amino acid
  VARIANT 720: note = X can be any amino acid
  VARIANT 725..726: note = X can be any amino acid
  VARIANT 731: note = X can be any amino acid
  VARIANT 733: note = X can be any amino acid
  VARIANT 736: note = X can be any amino acid
  VARIANT 750..752: note = X can be any amino acid
  VARIANT 754: note = X can be any amino acid
  VARIANT 764: note = X can be any amino acid
  VARIANT 768..769: note = X can be any amino acid
  VARIANT 781..782: note = X can be any amino acid
  VARIANT 785: note = X can be any amino acid
  VARIANT 788..789: note = X can be any amino acid
  VARIANT 802: note = X can be any amino acid
  VARIANT 804: note = X can be any amino acid
  VARIANT 807: note = X can be any amino acid
  VARIANT 811..812: note = X can be any amino acid
  VARIANT 814..815: note = X can be any amino acid
  VARIANT 817: note = X can be any amino acid
  VARIANT 821: note = X can be any amino acid
RXSGXXSKXVPGAYIFEFEXXQDTAXFYKXLNGEGSTRXKFXYKLFKGVSVQLKDLXNHEXKAQXMAXLPAVKNXWPVTLIXAPNPKVEWVAXSXXPXLXXRXXXXXXXPXDXSXFPXXQMXQIDKLRAKGXTGKGVXXAVIDTGIDYTHPALGGCFGXGCLVSFGTDLVGDXYXGFNTPVPDXDPXDCAGHGSHVAGIIAAQXNPYGFTGGAPXVTLGAYRVFGCXGQAGNDVLIXAYNXAFEDGAQIITASIGGPSGWAEEPWAXAVXRIVXAGVPCTVSAGNXGDXGLFXASTAANGKKVXAVASVDNXNIPXVXSXXXYKXDXGXXQXFGYVSSXKAWXGVXKPXYAVXXDTTIPXDXCXXLPDSTPDLSXYIVLVRRGTCTFVQKAQNVAAKGAKYLLYYNXXXGALXVDVSXXXXIXAXGMVXDXTGAXXXAAXKXXKTVTLXXXXXXXXXKQIQFXDNXXTGGALSXXTXWGPTWEXDVKPQISSPGGNILSTYPVAXGGYATLSGTSMACPLTAAAVALXXQARXTFDPXLXXNLLATTANPQXFNDGXXFYXFLAPVXQQGGGLIQAYDAAFATTLLSPSSLXFNDTDHFXXKXQITLKNTSKQRVTYKLNHVPTNTFYTLXXXXXYPAXFPNXAXAAHAXXXXXLXXVTLPAGXSITXDVXPXPPXXVDAXRLXLWSGYITVNGTDGXXLSVPYQGLTGSLHKQXVLXPXDSWIXXSTDEXLXPVXNGTVFTIPAPGNAXXXDXLPSLVVSPAXGSRXXRVDLVLLSAPPXXTKXKTXXFLDTTSIGQPAGXPXLWXSRGXXPXXWXGELXDNKFAPPGTYKAVFHALRIFGNEKKKEDWDVSESPAFTIKYA

SEQ ID NO: 26 - protein, 5 aa, synthetic construct
VIDTG

SEQ ID NO: 27 - protein, 11 aa, synthetic construct
HGSHVAGIIAA

SEQ ID NO: 28 - protein, 6 aa, synthetic construct
TASIGG

SEQ ID NO: 29 - protein, 7 aa, synthetic construct
  VARIANT 5: note = X can be any amino acid
SAGNXGD

SEQ ID NO: 30 - protein, 8 aa, synthetic construct
GTSMACPL

SEQ ID NO: 31-39 - skipped (9 entries)

SEQ ID NO: 40 - protein, 258 aa, unidentified
  source 1..258: note = T. harzianum
MAPVLAIASVLAALPALTMGAAITPRGSDIVGGTTAALGEFPYIVSLSTGGSHFCGGVLIDSRTVVTAGHCTIDQRASSVKVRAGTLTWASGGTQVGVSSLTLHPSYTVDSQGVPDNDVGVWHLATAIPTSSTIGYATLPASGSDPAAGTTLTVAGWGTTSENSNSLPSTLRKVSVPVVARATCDSDYDGEISNNMFCAAVAAGGKDSCSGDSGGPIIDPSGTLVGVVSWGQGCAERGFPGVYTRLGNYVSFINSNRG

SEQ ID NO: 41 - protein, 259 aa, unidentified
  source 1..259: note = Hypocrea jecorina
MAPASQVVSALMLPALALGAAIQPRGADIVGGTAASLGEFPYIVSLQNPNQGGHFCGGVLVNANTVVTAAHCSVVYPASQIRVRAGTLTWNSGGTLVGVSQIIVNPSYNDRTTDFDVAVWHLSSPIRESSTIGYATLPAQGSDPVAGSTVTTAGWGTTSENSNSIPSRLNKVSVPVVARSTCQADYRSQGLSVTNNMFCAGLTQGGKDSCSGDSGGPIVDANGVLQGVVSWGIGCAEAGFPGVYTRIGNFVNYINQNLA

SEQ ID NO: 42 - protein, 258 aa, unidentified
  source 1..258: note = T. guizhouense
MAPVLAIASVLAALPALTMGAAITPRGSDIVGGTTAALGEFPYIVSLSTGGSHFCGGVLIDSRTVVTAGHCTIDQRASSVKVRAGTLTWASGGTQVGVSSLTLHPSYTVDNQGVPDNDVGVWHLATAIPTSSTIGYATLPASGSDPAAGTTLTVAGWGTTSENSNSLPSTLRKVSVPVVARATCDSDYDGEISNNMFCAAVAAGGKDSCSGDSGGPIVDPSGTLVGVVSWGQGCAERGFPGVYTRLGNYVSFINSNRG

SEQ ID NO: 43 - protein, 254 aa, unidentified
  source 1..254: note = T. guizhouense
MAPVSQVVSALMLPALALGAAIEPRGADIVGGTTAALGEFPYIVSLSTQGSHFCGGVLVNANTVITAAHCSVDFSASQVKVRAGTLTWASGGTQVGVSRIIVNPSYNSRTTDFDVAVWKLASSIPESSTIGYATLPAAGSDPAAGSIVTTAGWGTTSEGSSSLPSTLRKVSVPVVSRATCQSEYGRSAVTTNMFCAAQAQGGKDSCQGDSGGPIVDANGVLQGLVSWGNGCAEAGFAGVYSRLGALVSFVNANL

SEQ ID NO: 44 - protein, 258 aa, unidentified
  source 1..258: note = T. harzianum
MASVLAIASVLAALPALTMGAAITPRGSDIVGGTTAALGEFPYIVSLSTGGSHFCGGVLIDSRTVVTAGHCTIDQRASSVKVRAGTLTWASGGTQVGVSSLTLHPSYTVDSQGVPDNDVGVWHLATAIPTSSTIGYATLPASGSDPAAGTTLTVAGWGTTSENSNSLPSTLRKVSVPVVARATCDNDYDGEISNNMFCAAVAAGGKDSCSGDSGGPIVDPSGTLVGVVPWGQGCAERGFPGVYTRLGNYVSFINSNRG

SEQ ID NO: 45 - protein, 259 aa, unidentified
  source 1..259: note = T. longibrachiatum
MAPASQVVSALMLPALALGAAIQPRGADIVGGTAASLGEFPYIVSLQNPSQGGHFCGGVLVNAKTVVTAAHCSVVYPASQIRVRAGTLTWNSGGTLVGVSQIIVNPSYNDRTTDFDVAVWHLSSSIPQSSTIGYATLPAQGSDPAAGSTVTTAGWGTTSENSNSIPSRLNKVSVPVVSRSTCQSDYRSQGLSVTNNMFCAGLTQGGKDSCSGDSGGPIVDANGVLQGVVSWGIGCAEAGFPGVYTRIGNFVNYINQNLA

SEQ ID NO: 46 - protein, 253 aa, unidentified
  source 1..253: note = T. parareesei
MAPASQVVSALMLPALALGAAIQPRGADIVGGTAASLGEFPYIVSLQNPNQGGHFCGGVLVNANTVVTAAHCSVVYPASQIRVRAGTLTWNSGGTLVGVSQIIVNPSYNDRTTDFDVAVWHLSSPIRESSTIGYATLPAQGSDPVAGSTVTTAGWGTTSENSNSIPSRLNKVSVPVVARSTCQADYRSQGLSVTNNMFCAGLTQGGKDSCSGDSGGPIVDANGVLQGVVSWGIGCAEAGFPAEAVGKIGLRCT

SEQ ID NO: 47 - protein, 259 aa, unidentified
  source 1..259: note = T. citrinoviride
MAPASQVVSALMLPALALGAAIQPRGADIVGGTAAAAGEFPYIVSLQNPNQGGHFCGGVLVNANTVVTAAHCSVVYPASQIRVRAGTLTWNSGGTQVGVSQIIVNPSYNDRTTDFDVAVWHLSSPLRESSTIGYATLPAQGSDPAAGSTLTTAGWGTTSENSNSIPSRLNKVSVPVVARATCQSDYRSQGLSVTNNMFCAGLTQGGKDSCSGDSGGPIVDANGVLQGVVSWGIGCAEAGFPGVYTRIGNFVNYINSNLA

SEQ ID NO: 48 - protein, 257 aa, unidentified
  source 1..257: note = T. arundinaceum
MAPVSALVSALMLPALALGAAIQPRGADIVGGTSAALGEFPYIVSLSNSNGHFCGGVLVNSRTVVTAGHCSVDTSASSVKVRAGTLTWASGGTQVGVSQIIVHPNYVVRSGGIPDFDVAVWHLSSPIAASSTIGYAKLTASGNDPASGSTVTTAGWGTTNENSNTLPARLLKVSVPVVSRTTCNSDYSGGVTNNMFCAGLTQGGKDSCSGDSGGPIVDASGTLIGVVSWGNGCAEAGFPGVYTRLGNFISFINANLA

SEQ ID NO: 49 - protein, 237 aa, synthetic construct
  VARIANT 4: note = X can be any amino acid
  VARIANT 8: note = X can be any amino acid
  VARIANT 15: note = X can be any amino acid
  VARIANT 17..18: note = X can be any amino acid
  VARIANT 28..34: note = X can be any amino acid
  VARIANT 42..45: note = X can be any amino acid
  VARIANT 48: note = X can be any amino acid
  VARIANT 51: note = X can be any amino acid
  VARIANT 54..58: note = X can be any amino acid
  VARIANT 61..63: note = X can be any amino acid
  VARIANT 72: note = X can be any amino acid
  VARIANT 77: note = X can be any amino acid
  VARIANT 82..86: note = X can be any amino acid
  VARIANT 88: note = X can be any amino acid
  VARIANT 90..97: note = X can be any amino acid
  VARIANT 99: note = X can be any amino acid
  VARIANT 102: note = X can be any amino acid
  VARIANT 105: note = X can be any amino acid
  VARIANT 107..112: note = X can be any amino acid
  VARIANT 120: note = X can be any amino acid
  VARIANT 122: note = X can be any amino acid
  VARIANT 124: note = X can be any amino acid
  VARIANT 126: note = X can be any amino acid
  VARIANT 129..130: note = X can be any amino acid
  VARIANT 132..134: note = X can be any amino acid
  VARIANT 136: note = X can be any amino acid
  VARIANT 143: note = X can be any amino acid
  VARIANT 145: note = X can be any amino acid
  VARIANT 147..149: note = X can be any amino acid
  VARIANT 151..152: note = X can be any amino acid
  VARIANT 154: note = X can be any amino acid
  VARIANT 162: note = X can be any amino acid
  VARIANT 164: note = X can be any amino acid
  VARIANT 167..169: note = X can be any amino acid
  VARIANT 171..179: note = X can be any amino acid
  VARIANT 185..188: note = X can be any amino acid
  VARIANT 195: note = X can be any amino acid
  VARIANT 203: note = X can be any amino acid
  VARIANT 205..206: note = X can be any amino acid
  VARIANT 208: note = X can be any amino acid
  VARIANT 210: note = X can be any amino acid
  VARIANT 212: note = X can be any amino acid
  VARIANT 214: note = X can be any amino acid
  VARIANT 217: note = X can be any amino acid
  VARIANT 222: note = X can be any amino acid
  VARIANT 225..232: note = X can be any amino acid
  VARIANT 234..237: note = X can be any amino acid
AAIXPRGXDIVGGTXAXXGEFPYIVSLXXXXXXXHFCGGVLXXXXTVXTAXHCXXXXXASXXXVRAGTLTWXSGGTXVGVSXXXXXPXYXXXXXXXXDXDVXVWXLXXXXXXSSTIGYAXLXAXGXDPXXGXXXTXAGWGTTXEXSXXXPXXLXKVSVPVVXRXTCXXXYXXXXXXXXXNMFCAXXXXGGKDSCXGDSGGPIXDXXGXLXGXVXWGXGCAEXGFXXXXXXXXGXXXX

SEQ ID NO: 50 - protein, 8 aa, synthetic construct
  VARIANT 1..2: note = X can be any amino acid
XXHFCGGV

SEQ ID NO: 51 - protein, 14 aa, synthetic construct
  VARIANT 1: note = X can be any amino acid
  VARIANT 6: note = X can be any amino acid
  VARIANT 3: note = X can be any amino acid
  VARIANT 14: note = X can be any amino acid
XVXWGXGCAERGFX

SEQ ID NO: 52-59 - skipped (8 entries)

SEQ ID NO: 60 - protein, 404 aa, unidentified
  source 1..404: note = T. harzianum
MQTFGAFLVSFLAASGMAAALPTEGQKTASIEVTYNKNYVAHGPTALFKAKRKYGAPISDNLRAAVAAKHSHSKRQTGSANTNPSDSADDEYITNVSIGTPAQVLPLDFDTGSSDLWVFSSETPKSSASGHVTYSPSKSSTSKKLSGSTWSISYGDHSSSSGDVYTDVVSIGGFSVKTQAVESATKVSTQFVQDTVISGLVGLGFDVGNTVRPRAQKTWFSNAANSLAEPLFTADLRHQETGSYNFGFIDTSLATGTIGYTPADGSEGYWGFTATGYSVGGAKLNRNSITGIADTGTTLLLLPDNIVDAYYNNVDSAEYDDSQEGIVFDCDEDLPSFSFGVGGQTITISGDLLNLTPIKEGSSTCFGGLQSSSDIGINIFGDVALKAALVVFDLGNERLGFAQK

SEQ ID NO: 61 - protein, 405 aa, unidentified
  source 1..405: note = Hypocrea atroviridis
MQTFGAFLVSFLAASGLAAALPAEGQQKTISVPVKYNANHAPHGPTELFKAKRKFGAPISENLKNRVAEHKAAKLARRQTGSAPNHPSDSEDDEYITNVSIGTPAQVLPLDFDTGSSDLWVFSSETPSSSARGHTIYTPSKSSTSKKVSGASWSITYGDNSSSSGDVYTDTVTIGGFSVTGQAVESATKVSTQFVQDTSNSGLVGLALDSGNTVKPTKQKTWFSNASSHLAQPVFTADLNHQADGSYNFGFIDTTLASGPITYVPISTGNGFWEFTSASYAIGGGSSKKHSTDGIADTGTTLLLLDDTIVDAYYNQVESAQYDDSQEGYTFDCSEDLPSFSFAVGSSTITVPGSLINFAPVDGNTCFGGLQSDDGIGISIFGDVAIKAALVVFDLGNKRLGWAQK

SEQ ID NO: 62 - protein, 405 aa, unidentified
  source 1..405: note = T. gamsii
MQTFGAFLVSFLAASGLAAALPAEGQQKTISVPVKYNANHAPHGPTELFKAKRKFGAPISENLKNRVTEHKAAKLARRQTGSAPNHPSDSEDDEYITNVSIGTPAQVLPLDFDTGSSDLWVFSSETPSSSARGHTIYTPSKSSTSKKVSGASWSITYGDNSSSSGDVYTDTVSIGGFSVTGQAVESATKVSSQFVQDTSNSGLVGLALDSGNTVKPTKQKTWFSNASSHLASPVFTADLNHQADGSYNFGFIDTSLASGPITYVPISTGNGFWEFTSSSYAIGGGSSKKHSTDGIADTGTTLLLLDDTIVDAYYNQVQSAQYDDSQEGYTFDCSEDLPSFSFAVGSSTITVPGSLINFAPVDGNTCFGGLQSDDGIGISIFGDVAIKAALVVFDLGNKRLGWAQK

SEQ ID NO: 63 - protein, 407 aa, unidentified
  source 1..407: note = Hypocrea jecorina
MQTFGAFLVSFLAASGLAAALPTEGQKTASVEVQYNKNYVPHGPTALFKAKRKYGAPISDNLKSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 64 - protein, 404 aa, unidentified
  source 1..404: note = Hypocrea virens
MQTFGAFLVSFLAASGLAAALPAEGQKTASIEVSYNKHYVPHGPTALFKAKRKYNAPISEKLHAAVAAKKALTKRQTGSANTNPSDSADDEYITNVSIGTPAQVLPLDFDTGSSDLWVFSSETPKSSASGHVTYSPSKSSTAKKLSGASWSITYGDHSSSSGDVYTDKVTVGGFSVSSQAVESATKVSTQFVQDTVISGLLGLGFDVGNTVKPRAQKTWFSNAANSLAQPLFTADLKHQQTGSYNFGFIDTTLAKGPIGYTPADGSEGYWGFTATGYSVGGAKLNRNSITGIADTGTTLLLLDDDVVNAYYNNVESAQYDDSQQGVVFDCSEDLPSFSFGVGGTTITIPGSLINLTPVSDGSSTCFGGLQSSADIGINIFGDVALKAALVVFDLGNERLGWAQK

SEQ ID NO: 65 - protein, 404 aa, unidentified
  source 1..404: note = T. harzianum
MQTFGAFLVSFLAASGMAAALPTEGQKTASIEVAYNKNYVAHGPTALFKAKRKYGAPISDNLRAAVAAKHSLTKRQTGSANTNPSDSADDEYITSVSIGTPAQVLPLDFDTGSSDLWVFSSETPKSSASGHALYSPSKSSTAKKLSGSTWSIEYGDKSSSSGDVYTDIVTIGGFSVKTQAVESATKVSTQFVQDTVISGLVGLGFDIGNQVSPRPQKTWFSNAASSLAEPLFTADLRHQETGSYNFGFIDTSLAKGTIGYTPADGSEGYWGFTATGYSVGGAKLNRNSITGIADTGTTLLLLPDNIVDAYYNNVESAQYDDSQEGVVFDCSEDLPSFSFGVGGQTITISGDLLNLTPIEEGSSTCFGGLQSSADIGINIFGDVALKAALVVFDLGNERLGWAQK

SEQ ID NO: 66 - protein, 407 aa, unidentified
  source 1..407: note = T. parareesei
MQTFGAFLVSFLAASGLAAALPTEGQKTASVEVQYNKNYVPHGPTALFKAKRKYGAPISDNLRSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 67 - protein, 406 aa, unidentified
  source 1..406: note = T. citrinoviride
MQTFGAFLVSFLAASGLAAALPTEGQKTASVEVQYNKNYVPHGPTALFKAKRKYGAPISENLKSIVAAKQAKAIAKRQTGSAPTHPSDSADSEYVTSVSIGTPAQVLPLDFDTGSSDLWVFSSETPKSSATGHALYTPSKSSTSKKVSGATWSISYGDGSSSSGDVYTDKVTIGGFSVATQGVESATRVSTEFVQDTVISGLVGLAFDSGNQVRPRPQKTWFSNAASSLAEPVFTADLRHGENGSYNFGYIDTSVAKGPVAYTPVDNSQGFWGFTASGYSVGGGKLSRSSISGIADTGTTLLLLDDDIVDAYYANVRSAEYDNQQEGVVFDCDEDLPTFSFGVGSSTITIPGDLLNLTPIEEGSSTCFGGLQSSSDIGINIFGDVALKAALVVFDLGNERLGWAQK

SEQ ID NO: 68 - protein, 407 aa, unidentified
  source 1..407: note = T. orientale
MQTFGAFLVSFLAASGLAAALPTEGQKTASVEVQYNKNYVAHGPTALFKAKKKYGAPISDNLRSIVAAKQAKQALAKRQTGSAPNHPSDSADSEYITSVSIGTPAQVLPLDFDTGSSDLWVFSSETPKSSATGHAIYSPSKSSTSKKVSGASWSISYGDGSSSSGDVYTDKVTIGGFSVNTQGVESATRVSSEFVQDTVISGLVGLAFDSGNQVRPHPQKTWFSNAASSLAEPLFTADLRHGQNGSYNFGYIDTSVAKGPVAYTPVDNSQGFWEFTATGYAVGSGKLNRNSIDGIADTGTTLLLLDDDVVNAYYANVRSAEYDNQQEGVVFDCDEDLPSFSFGVGGSTITIPGNLLNLTPLEEGSSTCFGGLQSSSGIGINIFGDVALKAALVVFDLGNERLGWAQK

SEQ ID NO: 69 - protein, 404 aa, unidentified
  source 1..404: note = T. arundinaceum
MQTFGAFLVSFLAASGMAAALPTEGQKTASVEVTYNQNYAAHGPTQLYKAKRKYGAPISDNLKAIVANRKALIKRQTGSAPNHPSDSADDEYITNVSIGTPAQVLPLDFDTGSSDLWVFSSETPKSSASGHTIYTPSKSSTSKKLSGATWSIEYGDKSTSSGDVYTDKVTVGGFSVSTQAVESATKVSAQFVQDTANSGLLGLAFDSINTVSPRQQKTWFSNAANSLAQPLFTANLNHQATGSYNFGFIDTSLASGPINYVPVDNSQGFWGFTASGYSVGGGKLNRSSLSGIADTGTTLLLLPDAVVNAYYANVESAEYDDEQEGVVFDCSEDLPTFSFGVGSGTITIPGDLLNLTPIDSSGQTCYGGLQSSSDIGINIFGDVALKAALVVFDLGNERLGWAQK

SEQ ID NO: 70 - protein, 405 aa, unidentified
  source 1..405: note = T. asperellum
MQTFGAFLVSFLAASGMAAALPAEGQQKTISVPVIYNANHAPHGPSALYKAKKKFGAPISESLKNNVAQHKAAKLARRQTGSAPNHPSDSEDDEYITNVSIGTPAQVLPLDFDTGSSDLWVFSSETPSSQAKGHTLYNPTKSSTSKKLSGYSWTISYGDGSSSSGDVYTDTVSVGGFSVTGQAVESATKVSSEFVSDTSNSGLLGLALDSINTVSPKQQKTWFSNASSKLAQPVFTADLNHQASGSYNFGYIDTSLASGPISYVPISTANGFWEFTSASYAIGSGSTKKHSTDGIADTGTTLLLLDDTIVDAHYGQVSSAQYDNSQEGYTFDCDENLPSFTFAVGSSKITVPGSLINFAPVSGNTCFGGLQSNDGIGINIFGDVAIKAALVVFDLGNKRLGWAQK

SEQ ID NO: 71 - protein, 388 aa, synthetic construct
  VARIANT 3: note = X can be any amino acid
  VARIANT 7: note = X can be any amino acid
  VARIANT 10: note = X can be any amino acid
  VARIANT 12..13: note = X can be any amino acid
  VARIANT 15: note = X can be any amino acid
  VARIANT 18..22: note = X can be any amino acid
  VARIANT 26..27: note = X can be any amino acid
  VARIANT 29: note = X can be any amino acid
  VARIANT 33: note = X can be any amino acid
  VARIANT 35..36: note = X can be any amino acid
  VARIANT 41..42: note = X can be any amino acid
  VARIANT 44..46: note = X can be any amino acid
  VARIANT 48..58: note = X can be any amino acid
  VARIANT 72: note = X can be any amino acid
  VARIANT 74: note = X can be any amino acid
  VARIANT 77: note = X can be any amino acid
  VARIANT 79: note = X can be any amino acid
  VARIANT 109: note = X can be any amino acid
  VARIANT 111..113: note = X can be any amino acid
  VARIANT 116..117: note = X can be any amino acid
  VARIANT 119: note = X can be any amino acid
  VARIANT 121: note = X can be any amino acid
  VARIANT 126: note = X can be any amino acid
  VARIANT 129: note = X can be any amino acid
  VARIANT 132..133: note = X can be any amino acid
  VARIANT 135: note = X can be any amino acid
  VARIANT 137: note = X can be any amino acid
  VARIANT 141: note = X can be any amino acid
  VARIANT 143: note = X can be any amino acid
  VARIANT 152: note = X can be any amino acid
  VARIANT 154..155: note = X can be any amino acid
  VARIANT 161..162: note = X can be any amino acid
  VARIANT 164: note = X can be any amino acid
  VARIANT 170: note = X can be any amino acid
  VARIANT 173..174: note = X can be any amino acid
  VARIANT 177: note = X can be any amino acid
  VARIANT 180..181: note = X can be any amino acid
  VARIANT 185: note = X can be any amino acid
  VARIANT 188..189: note = X can be any amino acid
  VARIANT 191..192: note = X can be any amino acid
  VARIANT 194: note = X can be any amino acid
  VARIANT 196: note = X can be any amino acid
  VARIANT 198..199: note = X can be any amino acid
  VARIANT 208..210: note = X can be any amino acid
  VARIANT 213: note = X can be any amino acid
  VARIANT 215: note = X can be any amino acid
  VARIANT 219: note = X can be any amino acid
  VARIANT 221: note = X can be any amino acid
  VARIANT 223..225: note = X can be any amino acid
  VARIANT 232: note = X can be any amino acid
  VARIANT 236..237: note = X can be any amino acid
  VARIANT 239: note = X can be any amino acid
  VARIANT 241..243: note = X can be any amino acid
  VARIANT 245: note = X can be any amino acid
  VARIANT 247..251: note = X can be any amino acid
  VARIANT 253: note = X can be any amino acid
  VARIANT 255: note = X can be any amino acid
  VARIANT 258..260: note = X can be any amino acid
  VARIANT 262..263: note = X can be any amino acid
  VARIANT 265..271: note = X can be any amino acid
  VARIANT 273..274: note = X can be any amino acid
  VARIANT 287: note = X can be any amino acid
  VARIANT 289..290: note = X can be any amino acid
  VARIANT 292: note = X can be any amino acid
  VARIANT 294: note = X can be any amino acid
  VARIANT 296..297: note = X can be any amino acid
  VARIANT 299: note = X can be any amino acid
  VARIANT 302: note = X can be any amino acid
  VARIANT 305..306: note = X can be any amino acid
  VARIANT 308: note = X can be any amino acid
  VARIANT 310..311: note = X can be any amino acid
  VARIANT 315: note = X can be any amino acid
  VARIANT 317: note = X can be any amino acid
  VARIANT 320: note = X can be any amino acid
  VARIANT 322: note = X can be any amino acid
  VARIANT 324: note = X can be any amino acid
  VARIANT 327..329: note = X can be any amino acid
  VARIANT 332..333: note = X can be any amino acid
  VARIANT 335: note = X can be any amino acid
  VARIANT 337: note = X can be any amino acid
  VARIANT 339..340: note = X can be any amino acid
  VARIANT 342..347: note = X can be any amino acid
  VARIANT 350: note = X can be any amino acid
  VARIANT 356..358: note = X can be any amino acid
  VARIANT 362: note = X can be any amino acid
  VARIANT 369: note = X can be any amino acid
  VARIANT 381: note = X can be any amino acid
  VARIANT 385: note = X can be any amino acid
  VARIANT 65..67: note = X can be any amino acid
LPXEGQXKTXSXXVXYNXXXXXHGPXXLXKAKXKXXAPISXXLXXXVXXXXXXXXXXXRQTGSAXXXPSDSXDXEYXTXVSIGTPAQVLPLDFDTGSSDLWVFSSETPXSXXXGHXXYXPXKSSTXKKXSGXXWXIXYGDXSXSSGDVYTDXVXXGGFSVXXQXVESATXVSXXFVXDTXXSGLXGLXXDXXNXVXPXXQKTWFSNAXXXLAXPXFTAXLXHXXXGSYNFGXIDTXXAXGXXXYXPXXXXXGXWXFTXXXYXXGXXXXXXXSXXGIADTGTTLLLLXDXXVXAXYXXVXSAXYDXXQXGXXFDCXEXLPXFXFXVGXXXITXXGXLXNXXPXXXXXXTCXGGLQSXXXIGIXIFGDVAXKAALVVFDLGNXRLGXAQK

SEQ ID NO: 72 - protein, 5 aa, synthetic construct
FDTGS

SEQ ID NO: 73 - protein, 11 aa, synthetic construct
IADTGTTLLLL

SEQ ID NO: 74-79 - skipped (6 entries)

SEQ ID NO: 80 - protein, 386 aa, unidentified
  source 1..386: note = T. harzianum
MLFSSIAIVAAATAALASPVKPSAKTAALSVKRVSNVKSLKNIVQKGQARINKINGVKDIEARASGPATNEDVSYVASVTIGGKSWDLIVDTGSSNTWCGAQSSCEPSSTGKSTGGSVQVSYGSGSFSGTEYKDTVSFGGLTVTSQSVGAARSSSGFSGVDGIIGFGPVDLTEDTVSNANTVPTFLDNLYSQGSISTEVLGVSFKPESGSDSDDTNGELTLGGTDSSKYTGSLTYFSTLKSGSAAPYWGISIASFTYGSTTLASSATGIVDTGTTLIYIPTKAYNAFLSAAGGKTDSSSGLAVFSKAPTSNFAIKFGSTTYTLTPSQYLVPTSQYSFYGLSSGKYYAWINDGGSSGVNTIIGQKFLENYYSVFDTTNGRIGFATAA

SEQ ID NO: 81 - protein, 386 aa, unidentified
  source 1..386: note = T. harzianum
MLFSSIAIVAAATAALASPVKPSAKTAALSVKRISNVKSLKNIVQKGQARINKINGVKDLEARASGPATNEDVSYVASVTIGGQSWDLIVDTGSSNTWCGAQRSCEPSSTGKSTGGSVQVSYGSGSFSGTEYKDTVTFGGLTVTSQSVGAASSSSGFSGVDGIIGFGPVDLTEDTVSNANTVPTFLDNLYSQGSISTEVLGVSFKPESGSDSDDTNGELTLGGTDSSKYTGSLTYFSTLKSGSAAPYWGISIASFTYGSTTLASSATGIVDTGTTLIYIPTKAYNAFLSAAGGKTDSSSGLAVFSKAPTSNFAIKFGSTTYTLTPSQYLVPTSQYSFYGLSSGKYYAWINDGGSSGVNTIIGQKFLENYYSVFDTTNGRIGFATAA

SEQ ID NO: 82 - protein, 386 aa, unidentified
  source 1..386: note = T. harzianum
MLFSSIAVVAAATAALASPVKPNAKTAALSVKRVSNVKSLKNIVQKGQARINKINGAKDIEARASGPVTNEDVSYVASVTIGGQSWDLIVDTGSSNTWCGAQRSCEPSSTGKSTGGSVQVSYGSGSFSGTEYKDTVSFGGLTVTSQSVGAARSSSGFSGVDGIIGFGPVDLTEDTVSNANTVPTFLDNLYSQGSISTEVLGVSFKPESGSDSDDTNGELTLGGTDSSKYTGSLTYFSTLKSSSAAPYWGISIASFTYGSTTLASSATGIVDTGTTLIYIPTKAYNAFLSAAGGKTDSSSGLAVFSKAPTSNFAIKFGSTTYTLTPSQYLVPTSQYDFYGLSSGKYYAWINDGGSSGVNTIIGQKFLENYYSVFDTTNGRIGFATAA

SEQ ID NO: 83 - protein, 454 aa, unidentified
  source 1..454: note = T. asperellum
MEAIFNVQTKFRLERGLSKIRAIRNENYKRHGTKSYVYLLNRFGFEPTKPGPYFQGVQFHQRGLAHPDFHEARGGRVHKKKHLRKKVTPEGSTDPNGTETGEVGAEDQQNDSEYLCEVTIGTPGQTLLLDFDTGSSDLWVFSTELSSTIQKGHTVFNPKNSSTFKALTGQKWQISYGDGSSASGDCGSDNVTVGGLTIKNQTVELASTLAQQFSQGTGDGLLGLAWPSINTVTTDGKPTPANTSRCQHITQDDVPSEAQLFTAAFYSERDANAQSFYTFGYIDNDLVTASGQDIAWTDVDNSQGFWMFPSTSSSINGKAISQSGNSAIADTGTTLALVSDEVCDALYKAIPGAKYDDQQQGYVFPMSTDVSSLPEFKVSVGDTQFVIQPEDLAFAPADNSNWYGGVQSRGNNPFDILGDVFLKSVYAIFDQGNQRFGAVPKIQATQNLNSSSNQ

SEQ ID NO: 84 - protein, 453 aa, unidentified
  source 1..453: note = Hypocrea virens
MEAIFQAQAKFRLDRGLTKVKAIRNQNYKRHGTKSYVYLLNRFGFEPTKPGPYYQQHRIRQRGLAHPDFHAPVGGRVHHTKHLHKKVNKHGAPDATGTETGEVGAEDQQNDSEYLCEVTIGTPGQTLLLDFDTGSSDLWVFSTELSKTIQKGHSVFQPTSSSTFKKLPNQAWQISYGDGSSASGDCGSDNVTIGGLTIKNQTVELASKLAQQFAQGTGDGLLGLAWPAINTVATNGKSTPANTPVANMITQDDIPSDAELFTAAFYSERDANAQSFYTFGYIDTDLVSSSGQDIAWTDIDNSQGFWMFPSTSTSVNGKAISQSGNSAIADTGTTLALVSDEVCEALYNAIPGATYDDQQQGYVFPQSTDVSSLPEFKVSVGDTQFVIQPEDLAFAPADDNNWYGGIQSRGTNPFDILGDVFLKSIYAIFDQGNQRFGAVPKIQASQNLSSSAQ

SEQ ID NO: 85 - protein, 417 aa, synthetic construct
  VARIANT 1: note = X can be any amino acid
  VARIANT 3: note = X can be any amino acid
  VARIANT 6..30: note = X can be any amino acid
  VARIANT 32..35: note = X can be any amino acid
  VARIANT 37: note = X can be any amino acid
  VARIANT 39..41: note = X can be any amino acid
  VARIANT 43..53: note = X can be any amino acid
  VARIANT 55..56: note = X can be any amino acid
  VARIANT 58..63: note = X can be any amino acid
  VARIANT 65..73: note = X can be any amino acid
  VARIANT 75..76: note = X can be any amino acid
  VARIANT 78..80: note = X can be any amino acid
  VARIANT 85..91: note = X can be any amino acid
  VARIANT 93..94: note = X can be any amino acid
  VARIANT 100..101: note = X can be any amino acid
  VARIANT 103..135: note = X can be any amino acid
  VARIANT 137: note = X can be any amino acid
  VARIANT 141: note = X can be any amino acid
  VARIANT 144..146: note = X can be any amino acid
  VARIANT 148..151: note = X can be any amino acid
  VARIANT 153: note = X can be any amino acid
  VARIANT 155..156: note = X can be any amino acid
  VARIANT 161..163: note = X can be any amino acid
  VARIANT 165: note = X can be any amino acid
  VARIANT 167..168: note = X can be any amino acid
  VARIANT 170..175: note = X can be any amino acid
  VARIANT 177..181: note = X can be any amino acid
  VARIANT 184..185: note = X can be any amino acid
  VARIANT 187..203: note = X can be any amino acid
  VARIANT 205..206: note = X can be any amino acid
  VARIANT 208..214: note = X can be any amino acid
  VARIANT 216..228: note = X can be any amino acid
  VARIANT 230..231: note = X can be any amino acid
  VARIANT 233..244: note = X can be any amino acid
  VARIANT 246: note = X can be any amino acid
  VARIANT 248..249: note = X can be any amino acid
  VARIANT 251..256: note = X can be any amino acid
  VARIANT 258..272: note = X can be any amino acid
  VARIANT 274..278: note = X can be any amino acid
  VARIANT 280..283: note = X can be any amino acid
  VARIANT 285..289: note = X can be any amino acid
  VARIANT 291..294: note = X can be any amino acid
  VARIANT 296: note = X can be any amino acid
  VARIANT 303..311: note = X can be any amino acid
  VARIANT 313..315: note = X can be any amino acid
  VARIANT 317..318: note = X can be any amino acid
  VARIANT 320..322: note = X can be any amino acid
  VARIANT 324..329: note = X can be any amino acid
  VARIANT 332..342: note = X can be any amino acid
  VARIANT 344..347: note = X can be any amino acid
  VARIANT 349: note = X can be any amino acid
  VARIANT 351..355: note = X can be any amino acid
  VARIANT 357..367: note = X can be any amino acid
  VARIANT 369..376: note = X can be any amino acid
  VARIANT 378..383: note = X can be any amino acid
  VARIANT 385..390: note = X can be any amino acid
  VARIANT 392: note = X can be any amino acid
  VARIANT 394..395: note = X can be any amino acid
  VARIANT 398..400: note = X can be any amino acid
  VARIANT 402..403: note = X can be any amino acid
  VARIANT 406..407: note = X can be any amino acid
  VARIANT 409: note = X can be any amino acid
  VARIANT 411: note = X can be any amino acid
  VARIANT 413..417: note = X can be any amino acid
XPXKPXXXXXXXXXXXXXXXXXXXXXXXXXRXXXXKXLXXXVXXXXXXXXXXXGXXDXXXXXXGXXXXXXXXXDXXYXXXVTIGXXXXXXXLXXDTGSSXXWXXXXXXXXXXXXXXXXXXXXXXXXXXXXXXXXXQXSYGXGSXXXGXXXXDXVXXGGLTXXXQXVXXAXXXXXXFXXXXXDGXXGXXXXXXXXXXXXXXXXXPXXTXXXXXXXQXXXXXXXXXXXXXFXXEXXXXXXXXXXXXTXGXXDXXXXXXSXXXXXXXXXXXXXXXWXXXXXSXXXXGXXXXXSXXXXIXDTGTTLXXXXXXXXXAXXXAXXGXXXDXXXXXXVFXXXXXXXXXXXFXXXXGXTXXXXXPXXXXXXXXXXXFXXXXXXXXYXXXXXXGXXXXXXIXGXXFLXXXYXXFDXXNXRXGXXXXX

SEQ ID NO: 86 - protein, 6 aa, synthetic construct
  VARIANT 1: note = X can be any amino acid
XDTGSS

SEQ ID NO: 87 - protein, 8 aa, synthetic construct
  VARIANT 2: note = X can be any amino acid
IXDTGTTL

SEQ ID NO: 88-89 - skipped (2 entries)

SEQ ID NO: 90 - protein, 263 aa, unidentified
  source 1..263: note = Hypocrea atroviridis
CGTQTNSPEFLSSVAALQALEQTADAVDARQSGATANITIPVYMTAFVNTTNSQDILSDNDLKAQFDVLVESYAPLGITFTLKNTTRIVNDDYAMGFDYYNFNGIKAETKQGGYDTLNLYYVTNMDPTEWGWGSCTLPTSNITSDSLWARLDGCTLVSYTVPGGTNDGTTYKGQIAVHEVGHWLSLLHTFDGLSCDGPGDYIADTPQESASTLNVCPVGRDSCPDEPGLDPIQNFMDYSGEGCWSEFTPGQGVRMANCWWTMR

SEQ ID NO: 91 - protein, 288 aa, unidentified
  source 1..288: note = T. gamsii
MLASNLLLGASAYFGGRNWCGTQTNSPEFLSTVAALQASEQSSSAVHARQHAADANITIPVYMTAFVNTTNSQDILSDDDLKAQFDVLVDNYAPLGITFTLKNTTRIVNDDYARGFDYYNFNGIKAETKQGGYDTLNLYFVTNMDPTEWGWGSCTLPTSNITSDSLWARLDGCTLVSYTVPGGTNDGTTYKGQIAVHEVGHWLSLLHTFDGLSCDGPGDYIADTPQESANTLNVCPVGRDSCPDEPGLDPINNFMDYSGEGCWSEFTPGQAVRMANCWWTMRYPFSSV

SEQ ID NO: 92 - protein, 270 aa, unidentified
  source 1..270: note = T. longibrachiatum
MPIFALAITRGATDSSELRCGSTANAGFMALSKRLAEKEDDNPLLSLRSVAKINVDVHIHIVAESEDDAAGNLSDDVVHEQIKVLSSDYAPADIFFSLKSIDRTSNATWANGTDLHGMWHQLRKGTYQDLNLYFVPTLQYYGFCSMPTTGDDLVDSVWQDGCTIRSDTVPGGSAVNYNLGKTVTHEVGHWFGLWHTFEGGCDGDGDYVADTPAQAAASDGCPPEGQDSCPNQPGKDAFHNFMDYSFDACYREFTPGQMNRMRRVWYAYRA

SEQ ID NO: 93 - protein, 264 aa, synthetic construct
  VARIANT 3..5: note = X can be any amino acid
  VARIANT 7..9: note = X can be any amino acid
  VARIANT 11..16: note = X can be any amino acid
  VARIANT 18..20: note = X can be any amino acid
  VARIANT 22..33: note = X can be any amino acid
  VARIANT 35..36: note = X can be any amino acid
  VARIANT 38..45: note = X can be any amino acid
  VARIANT 47..56: note = X can be any amino acid
  VARIANT 60..64: note = X can be any amino acid
  VARIANT 66..67: note = X can be any amino acid
  VARIANT 70..72: note = X can be any amino acid
  VARIANT 76..77: note = X can be any amino acid
  VARIANT 79: note = X can be any amino acid
  VARIANT 81: note = X can be any amino acid
  VARIANT 84..86: note = X can be any amino acid
  VARIANT 88..89: note = X can be any amino acid
  VARIANT 91..93: note = X can be any amino acid
  VARIANT 95: note = X can be any amino acid
  VARIANT 97: note = X can be any amino acid
  VARIANT 99..103: note = X can be any amino acid
  VARIANT 105..111: note = X can be any amino acid
  VARIANT 113: note = X can be any amino acid
  VARIANT 115..116: note = X can be any amino acid
  VARIANT 121: note = X can be any amino acid
  VARIANT 123..132: note = X can be any amino acid
  VARIANT 134: note = X can be any amino acid
  VARIANT 136..137: note = X can be any amino acid
  VARIANT 140..145: note = X can be any amino acid
  VARIANT 148: note = X can be any amino acid
  VARIANT 150..152: note = X can be any amino acid
  VARIANT 157..158: note = X can be any amino acid
  VARIANT 160: note = X can be any amino acid
  VARIANT 166..173: note = X can be any amino acid
  VARIANT 175..178: note = X can be any amino acid
  VARIANT 185..186: note = X can be any amino acid
  VARIANT 188: note = X can be any amino acid
  VARIANT 192: note = X can be any amino acid
  VARIANT 194..195: note = X can be any amino acid
  VARIANT 199: note = X can be any amino acid
  VARIANT 203: note = X can be any amino acid
  VARIANT 208..210: note = X can be any amino acid
  VARIANT 212..217: note = X can be any amino acid
  VARIANT 219: note = X can be any amino acid
  VARIANT 221: note = X can be any amino acid
  VARIANT 226..227: note = X can be any amino acid
  VARIANT 230: note = X can be any amino acid
  VARIANT 232..234: note = X can be any amino acid
  VARIANT 241..243: note = X can be any amino acid
  VARIANT 245..246: note = X can be any amino acid
  VARIANT 253..254: note = X can be any amino acid
  VARIANT 257..259: note = X can be any amino acid
  VARIANT 261..263: note = X can be any amino acid
CGXXXNXXXFXXXXXXLXXXEXXXXXXXXXXXXAXXNXXXXXXXXAXXXXXXXXXXLSDXXXXXQXXVLXXXYAPXXIXFXLKXXXRXXNXXXAXGXDXXXXXGXXXXXXXGXYXXLNLYXVXXXXXXXXXXGXCXXPTXXXXXXDSXWXXXDGCTXXSXTVPGGXXXXXXXXGXXXXHEVGHWXXLXHTFXGXXCDGXGDYXADTPXXXAXXXXXXPXGXDSCPXXPGXDXXXNFMDYSXXXCXXEFTPGQXXRMXXXWXXXR

SEQ ID NO: 94 - protein, 11 aa, synthetic construct
  VARIANT 7..8: note = X can be any amino acid
  VARIANT 10: note = X can be any amino acid
HEVGHWXXLXH

SEQ ID NO: 95-99 - skipped (5 entries)

SEQ ID NO: 100 - protein, 437 aa, unidentified
  source 1..437: note = Hypocrea atroviridis
MKTVLPWALLALTQVTSGCLLPHERDETVMKPIVRRQGSNGTPIGTGDRFKSGATAPRGLGTQSSSTSFSTILNVNEIKSGLQGLVNTYGIQTFNTPYKTAQGATVIGAQIGGNGTCTNAYRTFINGNIHARERGSADSVLYFAADLLYANQNNVGLTYGSKSYTNAQVKEALSTGLVFIPLSNPDGVAYDQSTNSCWRKNRNSNSGGVDLNRNFDFLWDFTHLFSSSVQSSVASTSPSSETYHGTSAFSEAETKNIKWVFDTYSKIRWFVDLHSYAGDVLWNWGSDENQSQYSYMNFQNSSYSSVRGILTDTPSPGRGYGEYVPQAELNDKITAANRIAAGLTGGGGRSYDAFQSSDLYPTSGASDDYAYSRHFVDSSKSLVHSFTVEFGFPNNAASCPFYPTVSQYNSNLRATSAGFMEFLLAASDLGLGDATSC

SEQ ID NO: 101 - protein, 442 aa, unidentified
  source 1..442: note = Hypocrea virens
MKTFIPVALLALSNVGSGCLLPHERDEASGLKPLVRRQGSNGAPIGTGDRFSGGATAPRGLGTQSSSTSIGTILSAKEVLSGLQGLVNTYGIETFNTPYKTAQGATVVGAKVGGTGGSCTDAYRVFLNGNIHARERGAADSVLYFISDLLYANKNNVGLTYGSKTYTNAQVKTALAAGIVFIPLSNPDGVAYDQSTNSCWRKNRNSNSGQSPGVDLNRNFDFLWDFKNLFASSAQSSVGSTSPSSETYHGTSAFSEPETKNIKWVMDTYSKIRWFIDLHSYAGDVLWNWGSDENQSSYPTMNFLNGTYSKVRGILTDTPSPGKGYGEYLPQADSDTKISVAKRVASAMTSGGGKSYTAFQSANLYPTSGASDDYAYSRHFADPTKNLIHSYTVEFGFANNAASCPFYPTVASFNSNLRATSAGFMEFLLAATDAGLGDATTC

SEQ ID NO: 102 - protein, 438 aa, unidentified
  source 1..438: note = T. harzianum
MKTFIPFALLALSNVSSGCLLPHERDDAAGLKPLVRRQGSNGAPIGSGDRFSGGATAPRGLGTQSSSASIGTILNTKEIISGLQGLANTYGIETFNTPYKTQNGATVVGARVGGTGGNCTDAYRVYFNGNIHARERGSADSVLYFISDLLYANKNNVGLTYGSRSYTNAQVKTALAAGIVFVPLSNPDGVAYDQSSNSCWRKNRNPNGGVDLNRNFDFLWDFRNLFASSAQSSVASTSPSSETYHGTSAFSEPETKNIKWVMDTHSKIRWYIDLHSYAGDVLWSWGSDENQSSYPTMNFLNSTYSKVRGILTDTPSPGRGYGEYIPQADSDTQIAVAKRVASALTAGGGRSYTSFQSADLYPTSGASDDYAYSRHFADPTKNLIHAYTVEFGFENTAASCPFYPTVSTFNSNLRATSAGFMEFLLAATDNGLGDATTC

SEQ ID NO: 103 - protein, 438 aa, unidentified
  source 1..438: note = T. harzianum
MKTFIPFALLALSNVSSGCLLPHERDDAAGLKPLVRRQGSNGAPIGSGDRFSGGATAPRGLGTQSSSASIGTILNTKEVVSGLQGLANTYGIETFNTPYKTAQGATVVGARVGGTGGNCTDAYRVYFNGNIHARERGSADSVLYFISDLLYANKNNVGLTYGSRSYTNAQVKTALAAGIVFVPLSNPDGVAYDQSSNSCWRKNRNSNGGVDLNRNFDFLWDFRNLFASSAQSSVASTSPSSETYHGTSAFSEPETKNIKWVMDTHSKIRWYIDLHSYAGDVLWSWGSDENQSSYPTMNFLNGTYSKIRGILTDTPSPGRGYGEYLPQADSDTQIAVAKRVASALTAGGGRSYTAFQSADLYPTSGASDDYAYSRHFADPTKNLIHAYTVEFGFQNNAASCPFYPTVSTFNSNLRATSAGFMEFLLAATDNGLGDATTC

SEQ ID NO: 104 - protein, 442 aa, unidentified
  source 1..442: note = T. parareesei
MKTFIPVALLALSQATSACLLPHERDEASGLKPVVRRQSSNGTPIGTGDRFSGGSTAPRGLGTQSSSTSFSTILNVKEVTSGLQGLANTYGVQTFNTPYKTAQGATVVGAKVGGTGGNCTDAYRVFFNGNIHARERGSADSVLYFISDLLYANAHNTGLTYGSKTYSNAQVKTALAAGIVFIPLSNPDGVAYDQSTNSCWRKNRNPNSGQSPGVDLNRNFDFLWDFRNLFASSAQSSVGSTSPSSETYHGASAFSEPETKNIKWVFDTYSKVRWFVDLHSYAGDVLWNWGSDENQVDYPTMNFLNGTYNKVRGILTDTPSPGRGYGEYVPQADLDVKEAAAKRVASALTAGGGRSYTAFQSAQLYPTSGASDDYAYSRHFSDPTKNLIHSYTIEFGFANNAASCPFYPSVSQYNSNLRATSAGFMELLLAATDYGLGDATTC

SEQ ID NO: 105 - protein, 438 aa, unidentified
  source 1..438: note = T. harzianum
MKTFIPFALLALSNVSSGCLLPHERDDAAGMKPLVRRQGSNGAPIGSGDRFSGGATAPRGLGTQSSSASIGTILNTKEVISGLQGLANTYGIETFNTPYKTQNGATVVGARVGGTGGNCTDAYRVYFNGNIHARERGSADSVLYFISDLLYANKNNVGLTYGSRSYTNAQVKTALAAGIVFVPLSNPDGVAYDQSSNSCWRKNRNPNGGVDLNRNFDFLWDFKNLFASSAQSSVASTSPSSETYHGTSAFSEPETKNIKWVMDTHSKIRWYIDLHSYAGDVLWSWGSDENQSSYPTMNFLNSTYSKVRGILTDTPSPGRGYGEYIPQADSDTQIAVAKRVASALTAGGGRSYTSFQSADLYPTSGASDDYAYSRHFADPTKNLIHAYTVEFGFENTAASCPFYPTVSTFNSNLRATSAGFMEFLLAATDNGLGDATTC

SEQ ID NO: 106 - protein, 424 aa, synthetic construct
  VARIANT 9..13: note = X can be any amino acid
  VARIANT 16: note = X can be any amino acid
  VARIANT 21: note = X can be any amino acid
  VARIANT 25: note = X can be any amino acid
  VARIANT 29: note = X can be any amino acid
  VARIANT 34..35: note = X can be any amino acid
  VARIANT 37: note = X can be any amino acid
  VARIANT 50: note = X can be any amino acid
  VARIANT 52..53: note = X can be any amino acid
  VARIANT 57..59: note = X can be any amino acid
  VARIANT 61..62: note = X can be any amino acid
  VARIANT 69: note = X can be any amino acid
  VARIANT 74..75: note = X can be any amino acid
  VARIANT 84..85: note = X can be any amino acid
  VARIANT 90: note = X can be any amino acid
  VARIANT 93..94: note = X can be any amino acid
  VARIANT 97..98: note = X can be any amino acid
  VARIANT 100: note = X can be any amino acid
  VARIANT 103: note = X can be any amino acid
  VARIANT 107..109: note = X can be any amino acid
  VARIANT 120: note = X can be any amino acid
  VARIANT 128..129: note = X can be any amino acid
  VARIANT 136..137: note = X can be any amino acid
  VARIANT 139: note = X can be any amino acid
  VARIANT 146..147: note = X can be any amino acid
  VARIANT 149: note = X can be any amino acid
  VARIANT 155: note = X can be any amino acid
  VARIANT 158..159: note = X can be any amino acid
  VARIANT 161: note = X can be any amino acid
  VARIANT 164: note = X can be any amino acid
  VARIANT 178: note = X can be any amino acid
  VARIANT 188: note = X can be any amino acid
  VARIANT 190..194: note = X can be any amino acid
  VARIANT 209..210: note = X can be any amino acid
  VARIANT 213: note = X can be any amino acid
  VARIANT 216: note = X can be any amino acid
  VARIANT 221: note = X can be any amino acid
  VARIANT 233: note = X can be any amino acid
  VARIANT 239: note = X can be any amino acid
  VARIANT 248: note = X can be any amino acid
  VARIANT 251: note = X can be any amino acid
  VARIANT 254: note = X can be any amino acid
  VARIANT 257..258: note = X can be any amino acid
  VARIANT 270: note = X can be any amino acid
  VARIANT 278..279: note = X can be any amino acid
  VARIANT 281..282: note = X can be any amino acid
  VARIANT 286: note = X can be any amino acid
  VARIANT 288..289: note = X can be any amino acid
  VARIANT 291..293: note = X can be any amino acid
  VARIANT 305: note = X can be any amino acid
  VARIANT 311: note = X can be any amino acid
  VARIANT 315..322: note = X can be any amino acid
  VARIANT 324: note = X can be any amino acid
  VARIANT 326: note = X can be any amino acid
  VARIANT 328..330: note = X can be any amino acid
  VARIANT 332: note = X can be any amino acid
  VARIANT 336: note = X can be any amino acid
  VARIANT 339..340: note = X can be any amino acid
  VARIANT 344..345: note = X can be any amino acid
  VARIANT 363: note = X can be any amino acid
  VARIANT 365..366: note = X can be any amino acid
  VARIANT 368: note = X can be any amino acid
  VARIANT 370: note = X can be any amino acid
  VARIANT 372..373: note = X can be any amino acid
  VARIANT 375: note = X can be any amino acid
  VARIANT 380: note = X can be any amino acid
  VARIANT 382: note = X can be any amino acid
  VARIANT 391: note = X can be any amino acid
  VARIANT 393..395: note = X can be any amino acid
  VARIANT 409: note = X can be any amino acid
  VARIANT 414: note = X can be any amino acid
  VARIANT 416: note = X can be any amino acid
  VARIANT 423: note = X can be any amino acid
CLLPHERDXXXXXKPXVRRQXSNGXPIGXGDRFXXGXTAPRGLGTQSSSXSXXTILXXXEXXSGLQGLXNTYGXXTFNTPYKTXXGATVXGAXXGGXXGXCTXAYRXXXNGNIHARERGXADSVLYFXXDLLYANXXNXGLTYGSXXYXNAQVKXALXXGXVFXPLSNPDGVAYDQSXNSCWRKNRNXNXXXXXGVDLNRNFDFLWDFXXLFXSSXQSSVXSTSPSSETYHGXSAFSEXETKNIKWVXDTXSKXRWXXDLHSYAGDVLWXWGSDENQXXYXXMNFXNXXYXXXRGILTDTPSPGXGYGEYXPQAXXXXXXXXAXRXAXXXTXGGGXSYXXFQSXXLYPTSGASDDYAYSRHFXDXXKXLXHXXTXEFGFXNXAASCPFYPXVXXXNSNLRATSAGFMEXLLAAXDXGLGDATXC

SEQ ID NO: 107 - protein, 7 aa, synthetic construct
  VARIANT 3: note = X can be any amino acid
  VARIANT 5..6: note = X can be any amino acid
HFXDXXK

SEQ ID NO: 108-109 - skipped (2 entries)

SEQ ID NO: 110 - RNA, 220 nt, unidentified
  source 1..220: mol_type = other RNA; note = Trichoderma reesei
ccgagtttacaactcccaaaccccaatgtgaacgttaccaatctgttgcctcggcgggattctctgccccgggcgcgtcgcagccccggatcccatggcgcccgccggaggaccaactcaaactcttttttctctccgtcgcggcttccgtcgcggctctgttttacctttgctctgagcctttctcggcgaccctagcgggcgtctcgaaaatgaatca

SEQ ID NO: 111 - DNA, 141 bp, unidentified
  source 1..141: mol_type = other DNA; note = Hypocrea jecorina
cccaagtactatgtcaccgtcattggtatgttggcagccaacatctcattgcgtcgttgacacgtcaaactaacgatgccctcacagacgctcccggccaccgtgacttcatcaagaacatgatcactggtacttcccagg

SEQ ID NO: 112 - DNA, 755 bp, unidentified
  source 1..755: mol_type = other DNA; note = Trichoderma parareesei
attctcccttgcctatctgtccaacatttgtcgaccaaatgttgtgccgacaggtttttttcatcaccccgctttcttctacccctccgagcgacgcaaatttttttgctgccttacgatgggttttagtggggttgcatcgagcaaccccaccaatactctggccgctctgtcggatccttcgacaacagtcacctcagcacacgcgtcaccaacacagcagtctttgatccgcgatgctaaccatgttcccctccataggaagccgccgaactcggcaagggttccttcaagtacgcgtgggttcttgacaagctcaaggccgagcgtgagcgtggtatcaccatcgacattgccctctggaagttcgagactcccaagtactatgtcaccgtcattggtatgttggcagccaacatctcattgcgtcgttgacacgtcaaactaacgatgccctcacagacgctcccggccaccgtgacttcatcaagaacatgatcactggtacttcccaggccgactgcgctatcctcattatcgctgccggtactggtgagttcgaggctggtatctccaaggatggccagacccgtgagcacgctctgctcgcctacaccctgggtgtcaagcagctcatcgtcgccatcaacaagatggacactgccaactgggccgaggctcgttaccaggaaatcatcaaggagacttccaacttcatcaagaaggtcggcttcaaccccaaggccgttgctttc

SEQ ID NO: 113 - DNA, 397 bp, unidentified
  source 1..397: mol_type = other DNA; note = Trichoderma parareesei
ggggggttgtttacagggtgctgaccgagctgctctccaggacaaggacggcgatggtacgtgatggcgagtgacgcgacaacacacttattgccctctctacgaagccgcaccgaagcactttttgccgatcgatcactctctcgtcgactcgaatcatgatacatggacaagaaactgacaggcttgacctcgtaggccagatcaccaccaaggagctgggcaccgtgatgcgctctctcggccagaacccttccgagtcggagctgcaggacatgatcaacgaggttgacgccgacaacaacggttccatcgacttccctggtacgtgaattgttgggagatttggtggttgaggtacacgggctgacgtggagcggtgaagaatttctcacca

SEQ ID NO: 114 - DNA, 20 bp, synthetic construct
  source 1..20: mol_type = other DNA
catcgagaagttcgagaagg

SEQ ID NO: 115 - DNA, 18 bp, synthetic construct
  source 1..18: mol_type = other DNA
aacttgcaggcaatgtgg

SEQ ID NO: 116 - DNA, 22 bp, synthetic construct
  source 1..22: mol_type = other DNA
gagttcaaggaggccttctccc

SEQ ID NO: 117 - DNA, 20 bp, synthetic construct
  source 1..20: mol_type = other DNA
catctttctggccatcatgg

SEQ ID NO: 118-119 - skipped (2 entries)

SEQ ID NO: 120 - DNA, 180 bp, unidentified
  source 1..180: mol_type = other DNA; note = Trichoderma virens
ccgagtttacaactcccaaacccaatgtgaacgttaccaaactgttgcctcggcgggatctctgccccgggtgcgtcgcagccccggaccaaggcgcccgccggaggaccaaccaaaactcttattgtataccccctcgcgggttttttactatctgagccatctcggcgcccctcgtgg

SEQ ID NO: 121 - DNA, 614 bp, unidentified
  source 1..614: mol_type = other DNA; note = Hypocrea virens
atgggctctgtcgttctcccgcatctaaactcaggctggcacgtcgaccaggccattttatcgggttcgtcactctccttgccgttgctccatcttatttatcgtgacatgtgatgcagtgtgtgctgaccagttgcatagaagaggatcgtctcgtcgtcatccggttcggacgtgaccacgatcgggtaagaagagtcacttttactcttggatattttcccttactaactgccattaggactgcatgctgcaagatgaagtgctctacaagatagccgatcgcgtcaaaaactttgccgtcatttacctctgcgacattgacgaggtaggtttttcatctcatccatggagtaaaggccgcttttaactggaagtaggttcctgatttcaacgccatgtacgaactgtacgacccttgctctattctgttcttctttcgcaacaagcatatgatgtgcgattttggtaccggtaacaacaacaagcttaactgggtgctggaggataagcaagagctcattgacattattgaaacgatttaccgcggagcaaagaaaggtagaggtttggtggtcagccccaaggattacagcacgagacacagatactag

SEQ ID NO: 122 - protein, 143 aa, unidentified
  source 1..143: note = Trichoderma virens
MGSVVLPHLNSGWHVDQAILSEEDRLVVIRFGRDHDRDCMLQDEVLYKIADRVKNFAVIYLCDIDEVPDFNAMYELYDPCSILFFFRNKHMMCDFGTGNNNKLNWVLEDKQELIDIIETIYRGAKKGRGLVVSPKDYSTRHRY

SEQ ID NO: 123 - protein, 132 aa, unidentified
  source 1..132: note = Trichoderma virens
LPHLNSGWHVDQAILSEEDRLVVIRFGRDHDRDCMLQDEVLYKIADRVKNFAVIYLCDIDEVPDFNAMYELYDPCSILFFFRNKHMMCDFGTGNNNKLNWVLEDKQELIDIIETIYRGAKKGRGLVVSPKDY

SEQ ID NO: 124 - DNA, 21 bp, synthetic construct
  source 1..21: mol_type = other DNA
gaagaggatcgtctcgtcgtc

SEQ ID NO: 125 - DNA, 21 bp, synthetic construct
  source 1..21: mol_type = other DNA
tcaggaacctcgtcaatgtcg

SEQ ID NO: 126-129 - skipped (4 entries)

SEQ ID NO: 130 - DNA, 568 bp, unidentified
  source 1..568: mol_type = other DNA; note = Trichoderma harzianum
agggatcattaccgagtttacaactcccaaacccaatgtgaaccataccaaactgttgcctcggcggggtcacgccccgggtgcgtcgcagccccggaaccaggcgcccgccggagggaccaaccaaactcttttctgtagtcccctcgcggacgttatttcttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggacctcgggagcccctaagacgggatcccggccccgaaatacagtggcggtctcgccgcagcctctcctgcgcagtagtttgcacaactcgcaccgggagcgcggcgcgtccacgtccgtaaaacacccaacttctgaaatgttgacctcggatcaggtaggaatacccgctgaacttaa

SEQ ID NO: 131 - DNA, 141 bp, unidentified
  source 1..141: mol_type = other DNA; note = Trichoderma cf. viride
cccaagtactatgtcaccgtcattggtatgttttcgcttttcctcattgatacttggagaccaagattctaacgtgccgctctgtagacgctcccggtcaccgtgatttcatcaagaacatgatcactggtacttcccagg

SEQ ID NO: 132-139 - skipped (8 entries)

SEQ ID NO: 140 - DNA, 564 bp, unidentified
  source 1..564: mol_type = other DNA; note = Trichoderma viride
agggatcattaccgagtttacaactcccaaacccaatgtgaacgttaccaaactgttgcctcggcggggtcacgccccgggtgcgtcgcagccccggaaccaggcgcccgccggaggaaccaaccaaactctttctgtagtcccctcgcggacgtatttctttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccgggggatcggcgttggggatcgggacccctcacacgggtgccggcccctaaatacagtggcggtctcgccgcagcctctcctgcgcagtagtttgcacaactcgcaccgggagcgcggcgcgtccacgtccgtaaaacacccaactttctgaaatgttgacctcggatcaggtaggaatacccgctgaacttaa

SEQ ID NO: 141 - DNA, 149 bp, unidentified
  source 1..149: mol_type = other DNA; note = Trichoderma asperellum
cccaagtactatgtcaccgtcattggtatgttttggactcttctctctagctatcgacattccaagtccgccattctaacatgctcttcccacagacgctcccggtcaccgtgatttcatcaagaacatgatcactggtacctcccagg

SEQ ID NO: 142 - DNA, 19 bp, synthetic construct
  source 1..19: mol_type = other DNA
tccgtaggtgaacctgcgg

SEQ ID NO: 143 - DNA, 20 bp, synthetic construct
  source 1..20: mol_type = other DNA
gctgcgttcttcatcgatgc

SEQ ID NO: 144 - DNA, 20 bp, synthetic construct
  source 1..20: mol_type = other DNA
tcctccgcttattgatatgc

SEQ ID NO: 145 - DNA, 20 bp, synthetic construct
  source 1..20: mol_type = other DNA
gcatcgatgaagaacgcagc

SEQ ID NO: 146 - DNA, 18 bp, synthetic construct
  source 1..18: mol_type = other DNA
gtgagcgtggtatcacca

SEQ ID NO: 147 - DNA, 19 bp, synthetic construct
  source 1..19: mol_type = other DNA
gccatccttggagaccagc

SEQ ID NO: 148-149 - skipped (2 entries)

SEQ ID NO: 150 - DNA, 545 bp, unidentified
  source 1..545: mol_type = other DNA; note = Trichoderma harzianum
accgaatttacaactcccaaacccaatgtgaacgttaccaaagtgttgcctcggcgggatctctgaccccgggtgcgtcgcagccccggaccaaggcgcccgccggangaccaaccaaaactcttattgtataccccctcgcgggttttttttataatctgagccttctcggcgcctctcgtaggcgtttcgaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggccctgccttggcggtggccgtttccgaaatacagtggcggtttcgccgcagcctttcctgcgcagtagtttgcacactcgcatcgggagcgcggcgcgtccacagccgttaaacacccaacttctgaaatgttgacctcggat

SEQ ID NO: 151 - DNA, 591 bp, unidentified
  source 1..591: mol_type = other DNA; note = Hypocrea lixii
gcggagggatcattaccgagtttacaactcccaaacccaatgtgaacgttaccaaactgttgcctcggcgggatctctgccccgggtgcgtcgcagccccggaccaaggcgcccgccggaggaccaaccaaaactcttattgtataccccctcgcgggtttttttataatctgagccttctcggcgcctctcgtaggcgtttcgaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggccctgccttggcggtggccgtctccgaaatacagtggcggtctcgccgcagcctctcctgcgcagtagtttgcacactcgcatcgggagcgcggcgcgtccacagccgttaaacacccaacttctgaaatgttgacctcggatcaggtaggaatacccgctgaacttaagcatatca

SEQ ID NO: 152 - DNA, 968 bp, unidentified
  source 1..968: mol_type = other DNA; note = Trichoderma afroharzianum
cactggtacttcccaggccgattgcgctatcctcatcattgccgccggtactggtgagttcgaggctggtatctccaaggatggccagacccgtgagcacgctctgctcgcctacaccctgggtgttaagcagctcatcgttgccatcaacaagatggacactgccaactgggccgaggctcgttaccaggaaatcatcaaggagacttccaacttcatcaagaaggtcggcttcaaccccaaggctgttgctttcgtccccatctccggtttcaacggtgacaacatgctccagccctccaccaactgcccctggtacaagggctgggagaaggagaccaaggctggcaagttcaccggcaagaccctccttgaggccatcgactccatcgagccccccaagcgtcccacggacaagcccctccgtcttcccctccaggatgtctacaagatcggtggtattggaacagttcccgtcggccgtatcgagactggtgtcctcaagcccggtatggtcgtcactttcgctccctccaacgtcaccactgaagtcaagtccgtcgagatgcaccacgagcagctcgtcgagggtgttcccggtgacaacgttggtttcaacgtcaagaacgtttccgttaaggaaattcgccgtggtaacgttgccggtgactccaagaacgacccccccatgggtgccgcttctttcaccgctcaggtcatcgtcatgaaccaccctggccaggtcggtgccggctacgcccccgttcttgactgccacactgcccacattgcctgcaagttcgccgagctccaggagaagatcgaccgccgtaccggtaaggctaccgagactgcccccaagttcatcaagtccggtgactctgccatcgtcaagatgattccctccaagcccatgtgcgttgaggctttcaccgactaccctcccctgggtcgtttcgccgtccgtg

SEQ ID NO: 153 - DNA, 222 bp, unidentified
  source 1..222: mol_type = other DNA; note = Trichoderma longibrachiatum
ccgagtttacaactcccaaacccaatgtgaacgttaccaatctgttgcctcggcgggattctcttgccccgggcgcgtcgcagccccggatcccatggcgcccgccggaggaccaactccaaactctttttttctctccgtcgcggctcccgtcgcggctctgttttatttttgctctgagcctttctcggcgaccctagcgggcgtctcgaaaatgaatca

SEQ ID NO: 154 - protein, 4 aa, synthetic construct
NEQK

SEQ ID NO: 155 - protein, 4 aa, synthetic construct
NHQK

SEQ ID NO: 156 - protein, 4 aa, synthetic construct
NDEQ

SEQ ID NO: 157 - protein, 4 aa, synthetic construct
QHRK

SEQ ID NO: 158 - protein, 4 aa, synthetic construct
MILV

SEQ ID NO: 159 - protein, 4 aa, synthetic construct
MILF

SEQ ID NO: 160 - protein, 4 aa, synthetic construct
STNK

SEQ ID NO: 161 - protein, 4 aa, synthetic construct
STPA

SEQ ID NO: 162 - protein, 4 aa, synthetic construct
SGND

SEQ ID NO: 163 - protein, 6 aa, synthetic construct
SNDEQK

SEQ ID NO: 164 - protein, 6 aa, synthetic construct
NDEQHK

SEQ ID NO: 165 - protein, 6 aa, synthetic construct
NEQHRK

SEQ ID NO: 166 - protein, 5 aa, synthetic construct
FVLIM